SEQUENCE LISTING (72 sequences)

SEQ ID NO: 1 - DNA, 29 bp, synthetic construct
  misc_feature 1..29: note = direct repeat
  source 1..29: mol_type = other DNA
gttgtaccttacctatgaggaattgaaac

SEQ ID NO: 2 - DNA, 29 bp, synthetic construct
  misc_feature 1..29: note = direct repeat
  source 1..29: mol_type = other DNA
gtcgtactttacctaaaaggaattgaaac

SEQ ID NO: 3 - DNA, 29 bp, synthetic construct
  misc_feature 1..29: note = direct repeat
  source 1..29: mol_type = other DNA
gtaaaactttacctaaaaggaattgaaac

SEQ ID NO: 4 - DNA, 29 bp, synthetic construct
  misc_feature 1..29: note = direct repeat
  source 1..29: mol_type = other DNA
gtcggtctttacctaaaaggaattgaaac

SEQ ID NO: 5 - DNA, 29 bp, synthetic construct
  misc_feature 1..29: note = direct repeat
  source 1..29: mol_type = other DNA
gtaaatctttacctaaaaggaattgaaac

SEQ ID NO: 6 - DNA, 29 bp, synthetic construct
  misc_feature 1..29: note = direct repeat
  source 1..29: mol_type = other DNA
gtcaaactttacctaaaaggaattgaaac

SEQ ID NO: 7 - DNA, 29 bp, synthetic construct
  misc_feature 1..29: note = direct repeat
  source 1..29: mol_type = other DNA
gtacggctttacctaaaaggaattgaaac

SEQ ID NO: 8 - DNA, 29 bp, synthetic construct
  misc_feature 1..29: note = direct repeat
  source 1..29: mol_type = other DNA
gtaggtctttacctaaaaggaattgaaac

SEQ ID NO: 9 - DNA, 29 bp, synthetic construct
  misc_feature 1..29: note = direct repeat
  source 1..29: mol_type = other DNA
gttagtctttacctaaaaggaattgaaac

SEQ ID NO: 10 - DNA, 29 bp, synthetic construct
  misc_feature 1..29: note = direct repeat
  source 1..29: mol_type = other DNA
gttacgctttacctaaaaggaattgaaac

SEQ ID NO: 11 - DNA, 29 bp, synthetic construct
  misc_feature 1..29: note = direct repeat
  source 1..29: mol_type = other DNA
gtcacactttacctaaaaggaattgaaac

SEQ ID NO: 12 - DNA, 67 bp, synthetic construct
  source 1..67: mol_type = other DNA
aaagcaagcccgttcacaactacgnnnnnnnnnngatcggaagagcacacgtctgaactccagtcac

SEQ ID NO: 13 - DNA, 20 bp, synthetic construct
  source 1..20: mol_type = other DNA
cgtagttgtgaacgggcttg

SEQ ID NO: 14 - DNA, 33 bp, synthetic construct
  source 1..33: mol_type = other DNA
gtgactggagttcagacgtgtgctcttccgatc

SEQ ID NO: 15 - DNA, 37 bp, synthetic construct
  source 1..37: mol_type = other DNA
aaaggatcggaagagcacacgtctgaactccagtcac

SEQ ID NO: 16 - DNA, 67 bp, synthetic construct
  source 1..67: mol_type = other DNA
aaagatttgtaccaaggttcctagnnnnnnnnnngatcggaagagcacacgtctgaactccagtcac

SEQ ID NO: 17 - DNA, 20 bp, synthetic construct
  source 1..20: mol_type = other DNA
ctaggaaccttggtacaaat

SEQ ID NO: 18 - DNA, 751 bp, synthetic construct
  source 1..751: mol_type = other DNA
gagttgatagacaatgtaacccactcgtgcacctcgagcaactgatcttatagatacagcatcttttactttcctcgagtagcctagcataaccccgcggggcctcttcgggggtctcgcggggttttttgctataaaacgaaaggctcagtcgaaagactgggcctttcgttttatctgctaacaaagcccgaaaggaagctgagttggctgctgccaccgctgagcaataactagcataaccccttggggcctctaaacgggtcttgaggggttttttgctgaaaggaggaactatatccggatgtcttcatggtagtaccaagatacgaagacatagtggcggggaagcttatgttccatagcaaaaagtcggtcagtctcgtggctgaaatcatgagttccacaaaatggctgaaattcaaggaaaatcaggaatctcagaaaaacgatcgaccgactttttcgataaaatggttgcaaaaatgagaaaaatctgatttaatagaatctgaaaacagcggaaatgctgttgtcgtactttacctaaaaggaattgaaacgtccccgccaggttgaatccgatatttggaggtacgatggaacagtctgggtgggattgagaagagaaaagaaaaccgccgatcctgtccaccgcattactgcaaggtagtggacaagaccggcggtcttaagttttttggctgaagcggccgcctcatggttatggcagcactgcataattttctta

SEQ ID NO: 19 - DNA, 45 bp, synthetic construct
  source 1..45: mol_type = other DNA
ccggaacttgacaattaatcatccggctcgtataatgtgtggagg

SEQ ID NO: 20 - DNA, 45 bp, synthetic construct
  source 1..45: mol_type = other DNA
cactcctccacacattatacgagccggatgattaattgtcaagtt

SEQ ID NO: 21 - DNA, 46 bp, synthetic construct
  source 1..46: mol_type = other DNA
ccggattgacggctagctcagtcctaggtacagtgctagctctagt

SEQ ID NO: 22 - DNA, 46 bp, synthetic construct
  source 1..46: mol_type = other DNA
cactactagagctagcactgtacctaggactgagctagccgtcaat

SEQ ID NO: 23 - DNA, 46 bp, synthetic construct
  source 1..46: mol_type = other DNA
ccggatttacggctagctcagtcctaggtatagtgctagctctagt

SEQ ID NO: 24 - DNA, 46 bp, synthetic construct
  source 1..46: mol_type = other DNA
cactactagagctagcactatacctaggactgagctagccgtaaat

SEQ ID NO: 25 - DNA, 46 bp, synthetic construct
  source 1..46: mol_type = other DNA
ccggatttacggctagctcagtcctaggtacaatgctagctctagt

SEQ ID NO: 26 - DNA, 46 bp, synthetic construct
  source 1..46: mol_type = other DNA
cactactagagctagcattgtacctaggactgagctagccgtaaat

SEQ ID NO: 27 - DNA, 46 bp, synthetic construct
  source 1..46: mol_type = other DNA
ccggatttatagctagctcagcccttggtacaatgctagctctagt

SEQ ID NO: 28 - DNA, 46 bp, synthetic construct
  source 1..46: mol_type = other DNA
cactactagagctagcattgtaccaagggctgagctagctataaat

SEQ ID NO: 29 - DNA, 46 bp, synthetic construct
  source 1..46: mol_type = other DNA
ccggattgacagctagctcagtcctagggattgtgctagctctagt

SEQ ID NO: 30 - DNA, 46 bp, synthetic construct
  source 1..46: mol_type = other DNA
cactactagagctagcacaatccctaggactgagctagctgtcaat

SEQ ID NO: 31 - DNA, 46 bp, synthetic construct
  source 1..46: mol_type = other DNA
ccggatttacagctagctcagtcctagggactgtgctagctctagt

SEQ ID NO: 32 - DNA, 46 bp, synthetic construct
  source 1..46: mol_type = other DNA
cactactagagctagcacagtccctaggactgagctagctgtaaat

SEQ ID NO: 33 - DNA, 46 bp, synthetic construct
  source 1..46: mol_type = other DNA
ccggactgatagctagctcagtcctagggattatgctagctctagt

SEQ ID NO: 34 - DNA, 46 bp, synthetic construct
  source 1..46: mol_type = other DNA
cactactagagctagcataatccctaggactgagctagctatcagt

SEQ ID NO: 35 - DNA, 46 bp, synthetic construct; identical to SEQ ID NO: 33
  source 1..46: mol_type = other DNA

SEQ ID NO: 36 - DNA, 46 bp, synthetic construct; identical to SEQ ID NO: 34
  source 1..46: mol_type = other DNA

SEQ ID NO: 37 - DNA, 657 bp, synthetic construct
  source 1..657: mol_type = other DNA
gtgatctaactcgagtagcctagcataaccccgcggggcctcttcgggggtctcgcggggttttttgctataaaacgaaaggctcagtcgaaagactgggcctttcgttttatctgctaacaaagcccgaaaggaagctgagttggctgctgccaccgctgagcaataactagcataaccccttggggcctctaaacgggtcttgaggggttttttgctgaaaggaggaactatatccggactgatagctagctcagtcctagggattatgctagctctagtagtggagaattaaattggaaaaagtcggtcgatctcatgcctgaaatcatgaattccgcaaaatggcggaaatttaaggaaaatcaggaatctcagaaaaacgatcgaccgacttttgtgataaaatggttgcaaaaaagagaaaaatttgatttaatagaatgtgaaaatagcggaaatgctgatgttgtaccttacctatgaggaattgaaacgtccccgccaggttgaatccgatatttggaggtacgatggaacagtctgggtgggattgagaagagaaaagaaaaccgccgatcctgtccaccgcattactgcaaggtagtggacaagaccggcggtcttaagttttttggctgaagcggccgctattct

SEQ ID NO: 38 - DNA, 67 bp, synthetic construct
  source 1..67: mol_type = other DNA
aaagctaatataccaccagcagtannnnnnnnnngatcggaagagcacacgtctgaactccagtcac

SEQ ID NO: 39 - DNA, 20 bp, synthetic construct
  source 1..20: mol_type = other DNA
tactgctggtggtatattag

SEQ ID NO: 40 - DNA, 67 bp, synthetic construct
  source 1..67: mol_type = other DNA
tgagattacgatcgccaggtcatgnnnnnnnnnngatcggaagagcacacgtctgaactccagtcac

SEQ ID NO: 41 - DNA, 20 bp, synthetic construct
  source 1..20: mol_type = other DNA
catgacctggcgatcgtaat

SEQ ID NO: 42 - DNA, 53 bp, synthetic construct
  source 1..53: mol_type = other DNA
acactctttccctacacgacgctcttccgatctnncctaaaaggaattgaaac

SEQ ID NO: 43 - DNA, 54 bp, synthetic construct
  source 1..54: mol_type = other DNA
acactctttccctacacgacgctcttccgatctnnncctaaaaggaattgaaac

SEQ ID NO: 44 - DNA, 55 bp, synthetic construct
  source 1..55: mol_type = other DNA
acactctttccctacacgacgctcttccgatctnnnncctaaaaggaattgaaac

SEQ ID NO: 45 - DNA, 56 bp, synthetic construct
  source 1..56: mol_type = other DNA
acactctttccctacacgacgctcttccgatctnnnnncctaaaaggaattgaaac

SEQ ID NO: 46 - DNA, 57 bp, synthetic construct
  source 1..57: mol_type = other DNA
acactctttccctacacgacgctcttccgatctnnnnnncctaaaaggaattgaaac

SEQ ID NO: 47 - DNA, 58 bp, synthetic construct
  source 1..58: mol_type = other DNA
acactctttccctacacgacgctcttccgatctnnnnnnncctaaaaggaattgaaac

SEQ ID NO: 48 - DNA, 59 bp, synthetic construct
  source 1..59: mol_type = other DNA
acactctttccctacacgacgctcttccgatctnnnnnnnncctaaaaggaattgaaac

SEQ ID NO: 49 - DNA, 54 bp, synthetic construct
  source 1..54: mol_type = other DNA
acactctttccctacacgacgctcttccgatctnnacctatgaggaattgaaac

SEQ ID NO: 50 - DNA, 55 bp, synthetic construct
  source 1..55: mol_type = other DNA
acactctttccctacacgacgctcttccgatctnnnacctatgaggaattgaaac

SEQ ID NO: 51 - DNA, 56 bp, synthetic construct
  source 1..56: mol_type = other DNA
acactctttccctacacgacgctcttccgatctnnnnacctatgaggaattgaaac

SEQ ID NO: 52 - DNA, 57 bp, synthetic construct
  source 1..57: mol_type = other DNA
acactctttccctacacgacgctcttccgatctnnnnnacctatgaggaattgaaac

SEQ ID NO: 53 - DNA, 58 bp, synthetic construct
  source 1..58: mol_type = other DNA
acactctttccctacacgacgctcttccgatctnnnnnnacctatgaggaattgaaac

SEQ ID NO: 54 - DNA, 59 bp, synthetic construct
  source 1..59: mol_type = other DNA
acactctttccctacacgacgctcttccgatctnnnnnnnacctatgaggaattgaaac

SEQ ID NO: 55 - DNA, 60 bp, synthetic construct
  source 1..60: mol_type = other DNA
acactctttccctacacgacgctcttccgatctnnnnnnnnacctatgaggaattgaaac

SEQ ID NO: 56 - DNA, 57 bp, synthetic construct
  source 1..57: mol_type = other DNA
aaagcactttggttatagaagagggatcggaagagcacacgtctgaactccagtcac

SEQ ID NO: 57 - DNA, 56 bp, synthetic construct
  source 1..56: mol_type = other DNA
aaagtcccatgaatgttccacatgatcggaagagcacacgtctgaactccagtcac

SEQ ID NO: 58 - DNA, 53 bp, synthetic construct
  source 1..53: mol_type = other DNA
gtgactggagttcagacgtgtgctcttccgatccctcttctataaccaaagtg

SEQ ID NO: 59 - DNA, 52 bp, synthetic construct
  source 1..52: mol_type = other DNA
gtgactggagttcagacgtgtgctcttccgatcatgtggaacattcatggga

SEQ ID NO: 60 - DNA, 67 bp, synthetic construct
  source 1..67: mol_type = other DNA
aaagactttccgcacaaaccgtgannnnnnnnnngatcggaagagcacacgtctgaactccagtcac

SEQ ID NO: 61 - DNA, 67 bp, synthetic construct
  source 1..67: mol_type = other DNA
aaagacaatccgtcaagtcactagnnnnnnnnnngatcggaagagcacacgtctgaactccagtcac

SEQ ID NO: 62 - DNA, 67 bp, synthetic construct
  source 1..67: mol_type = other DNA
aaagtaaacgactacacccgctcgnnnnnnnnnngatcggaagagcacacgtctgaactccagtcac

SEQ ID NO: 63 - DNA, 67 bp, synthetic construct
  source 1..67: mol_type = other DNA
aaagcgatatcatcgtccctttgtnnnnnnnnnngatcggaagagcacacgtctgaactccagtcac

SEQ ID NO: 64 - DNA, 20 bp, synthetic construct
  source 1..20: mol_type = other DNA
tcacggtttgtgcggaaagt

SEQ ID NO: 65 - DNA, 20 bp, synthetic construct
  source 1..20: mol_type = other DNA
cgagcgggtgtagtcgttta

SEQ ID NO: 66 - DNA, 20 bp, synthetic construct
  source 1..20: mol_type = other DNA
acaaagggacgatgatatcg

SEQ ID NO: 67 - DNA, 20 bp, synthetic construct
  source 1..20: mol_type = other DNA
ctagtgacttgacggattgt

SEQ ID NO: 68 - DNA, 24 bp, synthetic construct
  source 1..24: mol_type = other DNA
aaagacgcaggaaacaggcttgat

SEQ ID NO: 69 - DNA, 20 bp, synthetic construct
  source 1..20: mol_type = other DNA
atcaagcctgtttcctgcgt

SEQ ID NO: 70 - DNA, 20 bp, synthetic construct
  source 1..20: mol_type = other DNA
gtactggcgtatgaatcacg

SEQ ID NO: 71 - DNA, 20 bp, synthetic construct
  source 1..20: mol_type = other DNA
cgaatcaggataatacccgg

SEQ ID NO: 72 - DNA, 22 bp, synthetic construct
  source 1..22: mol_type = other DNA
  modified_base >1: mod_base = OTHER; note = Hexachlorofluorescein
  modified_base >22: mod_base = OTHER; note = Black Hole Quencher 1
agcgatctgaagaaccaggaat

(19) United States
(12) Patent Application Publication
(10) Pub. No.: US 2025/0333731 A1
(43) Pub. Date: Oct. 30, 2025

(54) TRANSCRIPTIONALLY RECORDING CELL COMPOSITION AND METHOD FOR NON-INVASIVE ASSESSMENT OF GUT FUNCTION

(71) Applicants: ETH ZURICH, Zurich (CH); UNIVERSITÄT BERN, Bern (CH)

(72) Inventors: Randall Jeffrey PLATT, Basel (CH); Florian SCHMIDT, Basel (CH); Alejandro ASENSIO-CALAVIA, Basel (CH); Katherine Elizabeth GUZZETTA, Basel (CH); Jakob ZIMMERMANN, Bern (CH); Andrew James MACPHERSON, Bern (CH); Tanmay TANNA, Basel (CH)

(73) Assignees: ETH ZURICH, Zurich (CH); UNIVERSITÄT BERN, Bern (CH)

(21) Appl. No.: 18/860,717

(22) PCT Filed: Apr. 28, 2023
(86) PCT No.: PCT/EP2023/061243

§ 371 Date: Oct. 28, 2024

(30) Foreign Application Priority Data
Apr. 28, 2022 (EP) 22170659.1
Apr. 28, 2022 (EP) 22170662.5

(51) Int. Cl. C12N 15/113; C12N 1/20; C12N 9/12; C12N 9/22; C12Q 1/6869; C12Q 1/6897; C12R 1/19

(52) U.S. Cl. CPC C12N 15/113; C12N 1/20; C12N 9/1276; C12N 9/226; C12Q 1/6869; C12Q 1/6897; C12Y 207/07049; C12N 2310/20; C12Q 2600/118; C12R 2001/19

(57) ABSTRACT

The invention relates to a bacterial cell comprising a Cas1 RT fusion protein, a Cas2 protein and a CRISPR direct repeat (DR) sequence, wherein an RNA polymerase promoter in addition to the leader sequence is associated with the DR sequence. The invention further relates to a composition comprising two bacterial cell populations, each comprising a Cas1 RT fusion protein and Cas2 protein. The two cell types contain different versions of a CRISPR direct repeat (DR) sequence. The invention further relates to methods for analysis of transcription recording events of bacteria having passed through a subject's intestine, to assign a probability to the subject having a condition, such as malnutrition or inflammation of the intestine.

This application claims the right of priority of European Patent Applications EP22170659.1 filed 28 Apr. 2022, and EP22170662.5 filed 28 Apr. 2022, both of which are incorporated by reference herein.

The project leading to this application has received funding from the European Research Council (ERC) under the European Union's Horizon 2020 research and innovation programme (grant agreements No 851021, No 744257 and No 742195).

### FIELD

The present invention relates to compositions comprising bacterial cells that transgenically express a fusion protein of a Cas1 polypeptide and a reverse transcriptase, and a Cas2 polypeptide. The cells also comprise a CRISPR direct repeat (DR) and leader sequence; at least two populations exist within the composition comprising two distinct DR sequences and thus can be distinguished. The cells can be administered to a subject, and will record transcriptional events occurring during their passage into the DR sequence, on passing through the subject. Upon collection, these transcriptional events can be read out by sequencing and can be attributed to the different cell populations on account of the differing DR sequences.

### BACKGROUND

Engineered microorganisms harboring molecular recording technologies enable the conversion of cellular histories into heritable nucleotide sequence archives encoded in DNA. Current recording tools, based on recombinases, single-stranded DNA recombineering, CRISPR-Cas9, and CRISPR spacer acquisition, are emerging as valuable platforms for encoding diverse biological features into DNA, including environmental metabolite concentration, cell lineage, gene expression and horizontal gene transfer. These technologies can illuminate complex dynamic cellular processes but have largely only been deployed in vitro. An unmet need is to leverage molecular recording to engineer sentinel cells capable of traversing the gastrointestinal tract and report on microbial and host physiology in the otherwise inaccessible intestinal lumen. Current sentinel cell technologies use biosensors to report on single biomolecules, but fall considerably short of capturing the complexity of the mammalian gastrointestinal tract.

The contents of the mammalian intestine are exposed to and shape dramatically different luminal environments during transit and colonization. The microorganisms that inhabit the small intestine and colon adapt their gene expression to environmental changes associated with nutrients and pathological states. Metatranscriptomics from fecal bacteria are largely uninformative about adaptations to the proximal luminal environment, sampling of which requires invasive or disruptive procedures, or use of devices that cannot preserve transient signals. Since metabolites and RNA are short-lived, omics-based measurements of transient stimuli only yield a snapshot of highly dynamic processes. An integrated measure of intestinal function therefore requires a non-invasive system able to sample a wide range of conditions and to preserve proximal transient signals in fecal samples.

The inventor's overriding goal in this work was to use molecular recording to establish a scalable non-invasive sentinel cell system for assessing intestinal and microbial pathology. To achieve this, the inventors used Record-seq, which employs the CRISPR spacer adaptation complex of Fusicatenibacter saccharivorans (FsRT-Cas1-Cas2) to acquire snippets of cellular RNAs as DNA within CRISPR arrays that can later be sequenced to reveal memory of past microbial gene expression. Record-seq captures transcriptome-scale information about the intestinal environment, recording the differences in microbial host interactions according to diet, disease or alterations of microbiota composition.

Shipman et al. (Science 353 (6298), 2016 (https://doi.org/10.1126/science.aaf1175) mutated the Cas1 protein, which is only capable of recording DNA, to enable recording of two different signals. The authors altered the PAM-recognition domain of the Cas1 protein to record snippets of DNA barcoded by PAM sequence.

Sheth et al., (Science 358; 1457-1461 (2017) (https://doi.org/10.1126/science.aao0958) used the same DNA recording Cas1-Cas2 system as Shipman et al. to perform multiplexed recording of three inducible signals.

Based on the above-mentioned state of the art, the objective of the present invention is to provide means and methods to monitor the health of a subject by recording biological states of microbiological components of the subject's microbiome.

This objective is attained by the subject-matter of the independent claims of the present specification, with further advantageous embodiments described in the dependent claims, examples, figures and general description of this specification.

### SUMMARY OF THE INVENTION

The invention relates to a composition comprising two bacterial cell populations, each comprising a Cas1 RT fusion protein and Cas2 protein. The two cell types contain different versions of a CRISPR direct repeat (DR) sequence.

In a first aspect, the invention relates to a composition comprising two bacterial cell populations: one of first bacterial cells and one of second bacterial cells. Both, the first cell and the second cells comprise
i. a first transgene nucleic acid sequence encoding a fusion protein, the fusion protein comprising or essentially consisting of a reverse transcriptase polypeptide and a Cas1 polypeptide and
ii. a second transgene nucleic acid sequence encoding a Cas2 polypeptide.

The first and second transgene nucleic acid sequences are under transcriptional control of one or several promoter sequence. In a bicistronic arrangement, the promoter drives transcription of both sequences into one mRNA; alternatively, distinct first and second transgene promoter sequence are present. The first cells comprise a third transgene nucleic acid sequence comprising a first CRISPR direct repeat sequence (first DR sequence) and a CRISPR leader sequence; and the second cells comprise a third transgene nucleic acid sequence comprising a second CRISPR direct repeat sequence (second DR sequence) and a CRISPR leader sequence. The first and the second DR sequences differ in at least one nucleotide. The first and second DR sequences and said CRISPR leader sequence are specifically recognizable by an RT-Cas1-Cas2 complex formed by the expression products of said first transgene nucleic acid sequence and said second transgene nucleic acid sequence.

The invention further provides a bacterial cell comprising a reverse transcriptase-Cas1 fusion polypeptide, a Cas2 polypeptide, and a CRISPR DR and leader sequence. An RNA polymerase promoter, particularly a weak promoter, is associated with the DR sequence.

A second aspect of the invention relates to a bacterial cell comprising a first transgene nucleic acid sequence encoding a fusion protein comprising or essentially consisting of a reverse transcriptase polypeptide and a Cas1 polypeptide, a second transgene nucleic acid sequence encoding a Cas2 polypeptide, and a third transgene nucleic acid sequence comprising a CRISPR direct repeat sequence (DR sequence) and a CRISPR leader sequence.

The DR and CRISPR leader sequences are specifically recognized by a RT-Cas1-Cas2 complex formed by the expression products of said first transgene nucleic acid sequences and said second transgene nucleic acid sequence of this cell. The cell is characterized by a third transgene promoter, particularly a weak third transgene promoter, being associated with the DR sequence.

The invention further relates to methods for analysis of transcription recording events of bacteria having passed through a subject's intestine, to assign a probability to the subject having a condition, such as malnutrition or inflammation of the intestine.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or”, and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

Gene designations used herein are the designations commonly used for bacterial genes, particularly—where in doubt—in the context of E. coli gene designations. For example, eda refers to KHG/KDPG aldolase (EC: 4.1.3.16), which catalyzes retro-aldol cleavage of 2-keto-3-deoxy-6-phosphogluconate (KDPG) to pyruvate and D-glyceraldehyde-3-phosphate.

Bacterial genes are often designated by a three-lowercase-one-uppercase-letter code, for example gatY (D-tagatose-1,6-bisphosphate aldolase subunit GatY) or gatZ (D-tagatose-1,6-bisphosphate aldolase subunit GatZ). In the interest of brevity, the inventors have grouped genes by the three-lowercase designations and added a plurality of uppercase specifiers. Thus gatYZ should be read as “gatY and/or gatZ”.

### DETAILED DESCRIPTION OF THE INVENTION

A first aspect of the invention relates to a composition comprising a first bacterial cell and a second bacterial cell, wherein both, the first cell and the second cell both comprise a first transgene nucleic acid sequence encoding a fusion protein, the fusion protein comprising or essentially consisting of a reverse transcriptase polypeptide and a Cas1 polypeptide, and a second transgene nucleic acid sequence encoding a Cas2 polypeptide.

RT-Cas1 as employed in the invention can be a natural fusion protein, used in the form found in a microbial genome. The inventors did not artificially fuse RT and Cas1 proteins. Also, the RT-Cas1 and Cas2 proteins are encoded next to one another in the same genomic neighbourhood (CRISPR locus) meaning they likely function together. Preferred embodiments use RT-Cas1 and Cas2 components of the same origin. In particular embodiments, the RT-Cas1 and Cas2 proteins are derived from a bacterium comprised in the group consisting of Fusicatenibacter saccharivorans, Azospirillum lipoferum, Micromonospora rosaria, Teredinibacter turnerae T8412, Woodsholea maritima, Desulfarculus baarsii, Cellulomonas bogoriensis, Candidatus accumulibacter sp., Oscillatoriales cyanobacterium, Rivularia sp. PCC 7116. In more particular embodiments, the RT-Cas1 and Cas2 proteins are derived from a bacterium comprised in the group consisting of Fusicatenibacter saccharivorans, Azospirillum lipoferum, Micromonospora rosaria. Of note, the Cas1-RT system may additionally include a Cas6 maturase, making it a Cas6-Rt-Cas1 system. This is the case for example for the Candidatus accumulibacter sp and Teredinibacter turnerae T8412 Cas1-RT polypeptides. See also Wang et al., Nature Communications volume 12, Article number: 2571 (2021).

Of note, there is at least one Cas2-less CRISPR integration complex (Wright et al., Mol Cell. 2019 Feb. 21; 73 (4): 727-737.e3), suggesting that there may be RT-Cas1 systems that function without Cas2. To the inventors' knowledge, however, there is only one report on this type of system and none fused to a reverse transcriptase.

The first transgene nucleic acid sequence and said second transgene nucleic acid sequence are under transcriptional control of a promoter. This can be the same promoter, or distinct first and second transgene promoter sequences are present. In certain embodiments, the first and second promoter are one and the same promoter sequence, i.e. the first transgene nucleic acid sequence and the second transgene nucleic acid sequence are on a bicistronic construct, the fusion protein of the reverse transcriptase and Cas1 polypeptide, and a second transgene nucleic acid sequence encoding a Cas2 polypeptide.

The first cell comprises a third transgene nucleic acid sequence comprising a first CRISPR direct repeat sequence (first DR sequence) and a CRISPR leader sequence.

The second cell comprises a third transgene nucleic acid sequence comprising a second CRISPR direct repeat sequence (second DR sequence) and a CRISPR leader sequence.

The first and the second DR sequences differ in at least one nucleotide.

The first and second DR sequences and the CRISPR leader sequence are specifically recognizable by an RT-Cas1-Cas2 complex formed by the expression products of the first transgene nucleic acid sequence and said second transgene nucleic acid sequence. The difference in the DR sequence serves as a type of molecular “barcode” to identify which cell recorded a transcript. The first and second transgene nucleic acid sequences, encoding the Cas1-RT and Cas-2 proteins necessary to perform the recording, can be the same for both first and second cell populations, but the third transgene, which carries the DR sequence into which transcripts are recorded, needs to be different in order for the two populations to be distinguishable.

The compositions of the invention are not limited to two distinct cells as specified above. Compositions comprising two distinct cell types having two different, distinguishable recorder sequences are the minimal set. The inventors contemplate compositions of two, three, four, five, six, seven, eight, nine or ten distinct cell types.

In certain embodiments, the first and, where present, second transgene promoter are an inducible promoter. Inducible promoters are helpful but not strictly required to practice the invention. The inventors contemplate promoters that are active in all or specific parts of the intestine. For example, promoters that are induced in low oxygen conditions or in response to specific signals present in the small intestine.

Inducible promoters for the first/second transgenes (RT-Cas1-Cas) could alter where recording occurs. Inducible promoters for the third/fourth sequences (leader-DR), if present, alter the recording efficiency.

In certain embodiments, the first and second, and possibly more, cell types are selected from bacteria that differ in their capability to occupy specific intestinal niches, each different bacterial cell (type) potentially being equipped with a different site-specific promoter. As a non-limiting example, one mutant is adapted to acidic, oxygen-replete small intestine and another one that is adapted to less acidic but anaerobic large intestine. The aim of both approaches is to obtain space-resolved records.

The inventors have found that one single direct repeat (DR) sequence works best in the combinations established so far, as CRISPR spacer acquisition is rare and thus, distinguishing CRISPR arrays that acquired a spacer from those that did not is useful. The single DR accomplishes that. In a more efficient acquisition system, more than one DR sequences may be employed in one construct. In current embodiments, the transgenes are provided on plasmid DNA. For application of this technology in humans, it is more likely that all genetic elements (RT-Cas1, Cas2, CRISPR arrays, promoters, etc.) will be integrated within the genome of the bacteria.

The leader sequences can be the same for the first and second cell, or they can be different. Most of the experiments the inventors performed so far were using two different CRISPR arrays containing two different leader-DR pairs (leader1-DR1, leader2-DR2). In the future, they plan to use the same leader sequence and different DR sequences.

It would also be straightforward to multiplex further, from two cells to n cells. Sentinel cell diagnostics could consist of a cocktail of “barcoded” bacteria recording different features of the intestine.

In certain embodiments, the first DR sequence is SEQ ID NO 01 (GTTGTACCTTACCTATGAGGAATTGAAAC) and the second DR sequence is SEQ ID NO 02 (GTCGTACTTTACCTAAAAGGAATTGAAAC).

In certain embodiments, the first DR sequence is (GTTGTACCTTACCTATGAGGAATTGAAAC or GTCGTACTTTACCTAAAAGGAATTGAAAC) and the second DR sequence differs from the first DR sequence in at least one nucleotide. In certain particular embodiments, the second DR sequence differs from the first DR sequence in 4, 3, 2, or 1 nucleotide(s). In more particular embodiments, it differs in two nucleotides.

The inventors have found differences of two, three or four nucleotides to yield the best results as these are more reliably observed in a deep sequencing readout.

In certain embodiments, the first and the second DR sequence are selected from different sequences of the group of SEQ ID NO 01 to SEQ ID NO 11.

In certain embodiments, the first DR sequence and/or the second DR sequence are individually associated with a third transgene RNA polymerase promoter being located in close proximity, more particularly 5′ direction, of the leader sequence thereof.

Another aspect of the invention relates to a cell comprising
i. a first transgene nucleic acid sequence encoding a fusion protein comprising or essentially consisting of a reverse transcriptase polypeptide and a Cas1 polypeptide,
ii. a second transgene nucleic acid sequence encoding a Cas2 polypeptide, and
iii. a third transgene nucleic acid sequence comprising a CRISPR direct repeat sequence (DR sequence) and a CRISPR leader sequence.

The DR sequence and the CRISPR leader sequence are specifically recognizable by an RT-Cas1-Cas2 complex formed by the expression products of said first transgene nucleic acid sequence and said second transgene nucleic acid sequence. and

Importantly, in this aspect of the invention, a third transgene promoter in addition to the leader sequence, is associated with (located in proximity of <1 kbp, particularly <500 bp), particularly located in 5′ direction of, the DR sequence.

In certain embodiments, the promoter is a weak third transgene promoter. In certain embodiments, the promoter is a weak constitutive third transgene promoter specific for RNA polymerase.

The inventors employed third transgene promoters in 5′ position to the DR (reading into the DR). Current results indicate that having the promoter on the 3′ end will also have a beneficial effect on recording efficacy. Without wanting to be bound by theory, the inventors assume that the mechanism of this effect is likely transcription-coupled repair, which recognizes DNA lesions and recruits DNA repair factors. Because lesions are created on both strands when recording, positioning the promoter up (5′) or down (3′) stream shouldn't impact the quality of the effect.

This third transgene promoter could be adjacent to the leader DR or several kilobases away for example the distance could be given as ≤5 kb. As the construct needs to be inserted into the genome of a bacterium, either as a plasmid, which immediately imposes size constraints, or as a construct for genomic integration, a closer proximity is of practical advantage. In certain embodiments, the third transgene promoter is located $1000 bp from the DR. In particular embodiments, the third transgene promoter is located≤500 bp from the DR. In more particular embodiments, the third transgene promoter is located≤200 bp from the DR.

In certain embodiments, the third transgene promoter is a weak RNA polymerase promoter. In certain particular embodiments, the third transgene promoter is a weak constitutive RNA polymerase promoter.

Bacteria have different types of promoters that recruit different cellular machinery but the inventors have not observed that different polymerases differentially activate transcription-coupled repair. What appears to be of importance for this effect is the strength of the promoter compared to the division rate of the bacterial cell. In the context of E. coli, a relatively weak promoter works best.

The inventors have found that promoter J23112 (Registry of Standard Biological Parts, http://parts.igem.org/Part: BBa J23112) worked best in their (E. coli based) exemplary system. Parts J23100 through J23119 are a family of constitutive promoter parts isolated from a small combinatorial library. J23119 is the “consensus” promoter sequence and the strongest member of the family. All parts except J23119 are present in plasmid J61002. Part J23119 is present in pSB1A2. This places the RFP downstream of the promoter. Reported activities of the promoters are given as the relative fluorescence of these plasmids in strain TG1 grown in LB media to saturation. See part BBa_J61002 for details on their use.

If no other indication is given herein, a “weak” promoter is defined as one having between 60% and 130% of the promoter activity when assayed in the red fluorescent expression assay as detailed in https://2019.igem.org/Team:Stuttgart/Contribution.

In certain embodiments, the first and the second cell are of the same species, particularly of the species E. coli.

In certain embodiments, the first and/or second transgene promoter sequence is an inducible promoter. In certain particular embodiments, the first and/or second transgene promoter sequence are one and the same promoter, i.e. the transcription is bicistronic.

In certain embodiments, the first and the second cell differ in expression of at least one gene. The first and second cell may also differ only in the sequence of their respective DR sequence, and at least one of the promoters. This might lead to the two, otherwise identical bacterial populations, to be sensitive to different inducible promoters.

The Cas1 proteins used by Shipman et al., or Sheth et al. (see above) are different from the ones used in the present invention, in that they require that the recorded sequence has a strict (protospacer adjacent motif, PAM) motif. The authors therefore altered the PAM-recognition domain of the Cas1 protein to record snippets of DNA barcoded by PAM sequence. The “recorded sequence” is technically called the protospacer. In the case of Shipman's system, the protospacer is derived from host or plasmid DNA and must contain a PAM sequence, which is a natural property (for self vs non-self-discrimination) of their DNA-recording Cas1-Cas2 system.

In certain embodiments, one gene the expression of which differs between the first and second bacterial population, encodes an enzyme catalyzing an essential metabolic step. An essential metabolic step is one without which the cell cannot function.

In certain embodiments, the first transgene nucleic acid sequence is under transcriptional control of a first promoter sequence and the second transgene nucleic acid sequence promoter is under transcriptional control of a second promoter sequence. In certain particular embodiments, each of said first and second promoter sequence is a different inducible promoter sequence.

In an alternative embodiment, the first and second transgenic nucleic acid sequence are under the expression of one and the same promoter.

Another aspect of the invention relates to a composition according to any one of the embodiments described above, for use in diagnosis.

A set of embodiments of this aspect relates to a method for monitoring of a diet of a patient or for diagnosis of a disease of a patient, particularly of a digestive or gastrointestinal disorder of a patient. This method comprises the steps of
collecting a composition of cells according as described for the first aspect of the invention from a feces sample collected from said patient, wherein said cells had been previously applied orally to said patient,
isolating the third transgene nucleic acid sequence from said cells, yielding isolated third transgene nucleic acid sequences, and
sequencing said isolated third transgene nucleic acid sequences, and distinguishing said isolated third transgene nucleic acid sequences derived from the first cell from the said isolated third transgene nucleic acid sequences derived from the second cell by the difference in the first and second DR sequence;
   thereby recording one or more transcripts of said composition of cells produced in the environment of the gastrointestinal tract.

Another aspect of the invention relates to a bacterial cell that comprises
i. a first transgene nucleic acid sequence encoding a fusion protein comprising or essentially consisting of a reverse transcriptase polypeptide and a Cas1 polypeptide,
ii. a second transgene nucleic acid sequence encoding a Cas2 polypeptide, and
iii. a third transgene nucleic acid sequence comprising a CRISPR direct repeat sequence (DR sequence) and a CRISPR leader sequence.

The DR sequence and the CRISPR leader sequence are specifically recognizable by an RT-Cas1-Cas2 complex formed by the expression products of the first transgene nucleic acid sequences and said second transgene nucleic acid sequence. Importantly, a third transgene promoter, particularly a weak third transgene promoter, is located in 5′ direction of the DR sequence, which greatly improves efficiency of the recording process.

In particular embodiments, the cassette consisting of the constitutive promoter, the leader and the DR is flanked by transcriptional terminators on both ends in 5′ and 3′ direction in order to prevent transcriptional read-through from neighbouring expression cassettes.

The following promoters were tested thus far with different impact on efficiency pTrc, BBa_J23100, BBa_J23106, BBa_J23110, BBa_J23115, BBa_J23117, BBa_J23109, BBa_J23112 (see FIG. 15B).

Another aspect of the invention relates to a cell as described in the preceding aspect, for use in diagnosis. This can also be formulated as the use of such cell in diagnosis.

Yet another aspect of the invention relates to a method for monitoring of a diet of a patient or for diagnosis of a disease of a patient, particularly of a digestive or gastrointestinal disorder of a patient.

The method comprising the steps of
collecting a plurality of cells according to the previously described aspect of the invention (specified in claim 14) from a feces sample collected from said patient; said cells had been previously applied orally to said patient,
isolating the third transgene nucleic acid sequence from these cells, yielding a plurality of isolated third transgene nucleic acid sequence, and
sequencing said isolated third transgene nucleic acid sequences, thereby recording one or more transcripts of said cell produced in the environment of the gastrointestinal tract.

With the current technology it is mostly one spacer per CRISPR array but the inventors have observed a small fraction with more than one. While cells have 5-15 CRISPR arrays on multi-copy plasmids the inventors expect that most cells have one spacer given the overall low efficiency of the process (1 spacer every 1-2 k cells).

Another alternative of this aspect of the invention relates to a method for diagnosis of a condition affecting the intestine of a patient, the condition being selected from the list comprising:
a. intestinal inflammation;
b. nutritional status of the patient (macronutrient insufficiency or excess; caloric adequacy after supplementation in malnutrition; micronutrient vitamin and mineral insufficiency), oxidative stress, intestinal inflammation, dysbiosis interaction between different taxa within the intestinal microbiota, niche adequacy for individual microbiota taxa, carbohydrate and lipid malabsorption (functional lactase, sucrase/isomaltase, trehalase and pancreas exocrine deficiencies), control for presence of gluten in diet of celiac disease patients, control for presence of FODMAPs (fermentable oligosaccharides, disaccharides, monosaccharides, and polyols) in diet of patients with intestinal functional disorders and bloating), bile salt malabsorption, delayed intestinal transit disorders (sclerosing conditions such as systemic sclerosis, neuromuscular disorders of the intestinal tract, intestinal changes in autonomic neuropathy of enteric neuropathies).

The method of this alternative of the diagnostic aspect of the invention comprises the steps:
i. In a first step, bacterial cells are collected, or bacterial DNA is collected, from a feces sample collected from said patient, wherein the bacterial cells had been previously applied orally to said patient; and wherein the bacterial cells prior to the oral application comprised:
   a first transgene nucleic acid sequence encoding a fusion protein comprising or essentially consisting of a reverse transcriptase polypeptide and a Cas1 polypeptide and a second transgene nucleic acid sequence encoding a Cas2 polypeptide, said first transgene nucleic acid sequence and said second transgene nucleic acid sequence being under transcriptional control of an, optionally inducible, promoter sequence, and
   a third transgene nucleic acid sequence comprising a CRISPR direct repeat sequence; said CRISPR direct repeat sequence being specifically recognizable by an RT-Cas1-Cas2 complex formed by the expression products of said first transgene nucleic acid sequence and said second transgene nucleic acid sequence,
In the collected bacterial cells, or in the bacterial DNA, spacers derived from a plurality of RNA molecules of the cell have been integrated into the CRISPR direct repeat sequence, yielding a modified third transgene nucleic acid sequence. The collected cell additionally comprises a modified third transgene nucleic acid sequence comprising one or more spacers, in other words transcripts recorded and inserted into the DR region by the Cas-1-RT-Cas2 construct, the spacers being derived from RNA molecules transcribed in the cell. The spacers are integrated into the CRISPR direct repeat sequence. As invariably, a plurality of cells is being subjected to analysis, a plurality of such spacer recorded sequences is available for subsequent analysis.
   ii. In a subsequent step, the modified third transgene nucleic acid sequence is isolated or amplified from said bacterial cells, yielding an isolated modified third transgene nucleic acid sequence, and
   iii. isolated modified third transgene nucleic acid sequences are determined (sequenced).

A high probability of having a condition affecting the intestine is assigned to said patient if,
when compared to a reference cell collected from a subject without (not having) said condition affecting the intestine
   or
after longitudinal studies of single patients before and following dietary or therapeutic interventions (such as correction or dietary deficiencies, prescription of diets lacking intolerance components, anti-inflammatory treatments, prokinetic administration, antibiotic usage, stool transplantation),
   the modified third transgene nucleic acid sequences isolated from said patient comprise a significantly different amount of spacer sequences, compared to the control, derived from a gene selected from the list comprising the following genes:
alaE; acrZ; bhsA; cpxP; glgS; Hha; ibpA; ibpB; lon; mqsA; mqsR; osmB; pspA; pspB; pspC; pspD; pspE; pspG; pspH; spy; tdcA; tdcB; tdcC; tdcD; tdcE; tdcF; tdcG; tomB; adhE; dmsA; dmsB; dmsC; dmsD; eda; edd; gadA; gadB; gadC; gadE; gntK; hdeA; hdeB; hdeD; hyaA; hyaB; hyaC; napA; napB; napC; napD; napF; napG; napH; narG; narH; narl; narJ; narK; narU; narW; narY; narZ; gntP; gntT; gntU; idnK; idnP; kdgK; kdgT; kduD; kdul; nagA; nagB; nagC; nagE; nagK; nagZ; nanA; nanC; nanE; nanK; nanM; nanQ; nanR; nanS; nanT; nanx; nanY; uxaA; uxaB; uxaC; uxuA; uxuB; uxuC; dkgA; gatY; gatZ; nhaA; nirB; nrfA; yeaD; yqhC; yqhD; zrAP; aroF; dgoA; dgoD; dgok; lacA; lacZ; IpxC; rbsD; rbsK; yefM.

In particular embodiments, the spacers used to determine a high probability of having a condition affecting the intestine, by difference to a control sample, are taken from the list comprising the following genes:
(Malnutrition upregulated): eda; edd; gatYZ; gntKPTU; idnKP; kdgKT; kduDI; nagABCEKZ; nanACEKMQRSTXY; nirB; uxaABC; uxuABC; zraP;
(Malnutition down regulated): alaE; dmsABCD; gadABC; gadABCE; hdeABD; hyaABC; lacAZ; napABCDFGH; narGIJKUWYZ
(Inflammation upregulated): (alaE); ipbAB; lon; mqsAR; pspABCDEGH; spy; tcdABCDEFG; tomB
(Inflammation downregulated): (adhE; dmsABCD; eda; edd); gad (ABC) E; (gntK; hdeABD; napABCDFGH); nar(G)H(IJKUWYZ); (gntTU; kdgKT); nagABCEKZ; (nanACEKMQRSTXY; uxaABC)
   (Double mentions in brackets).

In certain embodiments, the modified third transgene nucleic acid sequences isolated from said patient comprise a significantly different amount of a spacer derived from a gene selected from the list comprising gntU, gntT, kdgT, eda, edd, pspG, spy, mqsAR, hha, narL, narGH, gadC, kdpDE.

Rather than a single number of genes identified will be important for the analysis of the results generated by the method of the invention, the identity of the genes and pathways identified as active by the recording will inform a diagnosis. A ‘diagnosis’ will be reached after analyzing as much of an entire system/transcriptional response as is available.

In certain embodiments, a difference of 2-fold is used as a threshold (log2FoldChange>1 or <−1). However, other thresholds may apply, for example, if a gene has a lower difference but has high specificity in classifying patient groups in longitudinal studies.

Many extraintestinal conditions are associated to changes in the microbiota, hence the method according to the invention, which can detect dysbiosis, can indirectly obtain diagnostic evidence for diverse kinds of conditions. Such could, in addition to the conditions outlined above, include small intestinal bacterial overgrowth, infections, gastritis, cancers such as colon adenocarcinoma.

Another aspect of this invention includes compliance monitoring for pharmacological interventions and monitoring of drug metabolism and effects on the gut/microbiota.

In certain embodiments, the condition is inflammation of the intestine, and a high probability of the patient suffering from intestinal inflammation is assigned to said patient if, when compared to a reference cell collected from a subject without malnutrition, the isolated modified third transgene nucleic acid sequence comprises a significantly different amount of a spacer derived from a gene selected from the list:
alaE; acrZ; bhsA; cpxP; glgS; Hha; ibpAB; lon; mqsAR; osmB; pspABCDEGH; spy; tcdABCDEFG; tomB; adhE; dmsABCD; eda; edd; gadABCE; gntK; hdeA; hdeB; hdeD; hyaA; hyaB; hyaC; napABCDFGH; narGHIJKUWYZ; gntPTU; idnKP; kdgKT; kduDI; nagABCEKZ; nanACEKMQRSTXY; uxaABC; uxuABC.

In certain alternative embodiments, the condition is inflammation of the intestine, and a high probability of the patient suffering from intestinal inflammation is assigned to said patient if, when compared to a reference cell collected from a subject without malnutrition, the isolated modified third transgene nucleic acid sequence comprises a significantly different amount of a spacer derived from a gene selected from the list:
alaE; ipbAB; lon; mqsAR; pspABCDEGH; spy; tcdABCDEFG; tomB; adhE; dmsABCD; eda; edd; gadABCE; gntK; hdeABD; napABCDFGH; narGHIJKUWYZ; gntTU; kdgKT; nagABCEKZ; nanACEKMQRSTXY; uxaABC.

In certain embodiments, inflammation of the intestine is diagnosed if any one, or several, of the following indicator genes is upregulated, i.e. an increased amount of spacer acquisition, compared to the control, is found: alaE; ipbAB; lon; mqsAR; pspABCDEGH; spy; tcdABCDEFG; tomb.

In certain embodiments, inflammation of the intestine is diagnosed if any one, or several, of the following indicator genes is downregulated, i.e. an decreased amount of spacer acquisition, compared to the control, is found: adhE; dmsABCD; eda; edd; gadABCE; gntK; hdeABD; napABCDFGH; narGHIJKUWYZ; gntTU; kdgKT; nagABCEKZ; nanACEKMQRSTXY; uxaABC.

In certain embodiments, this aspect of the invention embodies a method for diagnosis of a nutritional status of a patient, or a method for the monitoring of the successful compliance with dietary therapy, caloric supplementation or reducing diets, or therapeutic correction of inflammatory or motility-dependent intestinal disease with secondary effects on intestinal absorptive function, the status being selected from the list comprising:
a. aloric insufficiency or excess;
b. micronutrient (mineral/vitamin) insufficiency
c. the presence in the lower intestine of nutrient components to which patients are intolerant (disaccharides, fermentable oligosaccharides, disaccharides, monosaccharides, and polyols, unabsorbed bile acids, gluten products, unabsorbed lipids)
d. pancreatic exocrine insufficiency
e. inflammatory or structural disease affecting intestinal absorptive function.

The method of this embodiment comprises the steps:
i. collecting sentinel cells from a feces sample collected from said patient, wherein the sentinells cells had been previously applied orally to said patient, and wherein the sentinel cells, prior to the oral application, comprised:
   a first transgene nucleic acid sequence encoding a fusion protein comprising or essentially consisting of a reverse transcriptase polypeptide and a Cas1 polypeptide and a second transgene nucleic acid sequence encoding a Cas2 polypeptide, said first transgene nucleic acid sequence and said second transgene nucleic acid sequence being under transcriptional control of an, optionally inducible, promoter sequence, and.
   a third transgene nucleic acid sequence comprising a CRISPR direct repeat sequence; said CRISPR direct repeat sequence being specifically recognizable by an RT-Cas1-Cas2 complex formed by the expression products of said first transgene nucleic acid sequence and said second transgene nucleic acid sequence,
      and wherein in the collected cells, a plurality of spacers derived from a plurality of RNA molecules of the cells have been integrated into the CRISPR direct repeat sequence, yielding modified third transgene nucleic acid sequences. The collected cells additionally comprise modified third transgene nucleic acid sequences comprising a plurality of spacers derived from a plurality of RNA molecules of the cell, wherein the spacers are integrated into the CRISPR direct repeat sequence;
ii. isolating and/or amplifying the modified third transgene nucleic acid sequences, yielding isolated modified third transgene nucleic acid sequences, and
iii. sequencing said isolated modified third transgene nucleic acid sequence.

A high probability of malnutrition is assigned to said patient if, when compared to sequences from reference cells collected from a subject without malnutrition, the isolated modified third transgene nucleic acid sequences comprise a significantly different amount of spacers derived from a gene selected from the list: adhE; bhsA; dkgA; eda; edd; gatY; gatZ; gntK; gntP; gntT; gntU; idnK; idnP; kdgK; kdgT; kduD; kdul; nagA; nagB; nagC; nagE; nagK; nagZ; nanA; nanC; nanE; nanK; nanM; nanQ; nanR; nanS; nanT; nanx; nanY; nhaA; nirB; nrfA; uxaA; uxaB; uxaC; uxuA; uxuB; uxuC; yeaD; yqhC; yqhD; zrAP; alaE; aroF; dmsA; dmsB; dmsC; dmsD; dgoA; dgoD; dgok; gadA; gadB; gadC; gadE; hdeA; hdeB; hdeD; hyaA; hyaB; hyaC; lacA; lacZ; IpxC; napA; napB; napC; napD; napF; napG; napH; narG; narH; narl; narJ; narK; narU; narW; narY; narZ; rbsD; rbsK; yefM.

In certain embodiments, a high probability of malnutrition is assigned to said patient if the modified third transgene nucleic acid sequence isolated from said patient, compared to the reference cell, comprises
a significantly higher amount of a spacer derived from a gene selected from the list comprising adhE; bhsA; dkgA; eda; edd; gatY; gatZ; gntK; gntP; gntT; gntU; idnK; idnP; kdgK; kdgT; kduD; kdul; nagA; nagB; nagC; nagE; nagK; nagZ; nanA; nanC; nanE; nanK; nanM; nanQ; nanR; nanS; nanT; nanx; nanY; nhaA; nirB; nrfA; uxaA; uxaB; uxaC; uxuA; uxuB; uxuC; yeaD; yqhC; yqhD; zrAP and/or
a significantly lower amount of a spacer derived from a gene selected from the list comprising alaE; aroF; dmsA; dmsB; dmsC; dmsD; dgoA; dgoD; dgok; gadA; gadB; gadC; gadE; hdeA; hdeB; hdeD; hyaA; hyaB; hyaC; lacA; lacZ; IpxC; napA; napB; napC; napD; napF; napG; napH; narG; narH; narl; narJ; narK; narU; narW; narY; narZ; rbsD; rbsK; yefM.

In certain embodiments, malnutrition of the patient is diagnosed if any one, or several, of the following indicator genes is upregulated, i.e. an increased amount of spacer acquisition, compared to the control, is found: eda; edd; gatYZ; gntKPTU; idnKP; kdgKT; kduDI; nagABCEKZ; nanACEKMQRSTXY; nirB; uxaABC; uxuABC; zraP.

In certain embodiments, malnutrition of the patient is diagnosed if any one, or several, of the following indicator genes is downregulated, i.e. an decreased amount of spacer acquisition, compared to the control, is found: alaE; dmsABCD; gadABC; gadABCE; hdeABD; hyaABC; lacAZ; napABCDFGH; narGIJKUWYZ.

Yet another aspect of the invention relates to an isolated DR nucleic acid molecule comprising a direct repeat sequence selected from the group comprising SEQ ID NO 1, SEQ ID NO 02, SEQ ID NO 03, SEQ ID NO 04, SEQ ID NO 05, SEQ ID NO 06, SEQ ID NO 07, SEQ ID NO 08, SEQ ID NO 09, SEQ ID NO 10, SEQ ID NO 11.

One distinction of the method according to the invention is that it does not require a priori knowledge of the expected signals, unlike the system disclosed by Sheth et al., where each multiplexed recorder is sensitive to only one single cue. In other words, each of the multiplexed recorders of the present invention is capable of recording the entire spectrum of the E. coli transcriptome.

Wherever alternatives for single separable features are laid out herein as “embodiments”, it is to be understood that such alternatives may be combined freely to form discrete embodiments of the invention disclosed herein.

The specification further encompasses the following items:

### Items:

1. A composition comprising a first cell and a second cell, wherein both, the first cell and the second cell comprise
i. a first transgene nucleic acid sequence encoding a fusion protein, the fusion protein comprising or essentially consisting of a reverse transcriptase polypeptide and a Cas1 polypeptide and
ii. a second transgene nucleic acid sequence encoding a Cas2 polypeptide,
wherein said first transgene nucleic acid sequence and said second transgene nucleic acid sequence are under transcriptional control of a promoter sequence, and
wherein the first cell comprises
iii. a third transgene nucleic acid sequence comprising a first CRISPR direct repeat sequence (first DR sequence) and a CRISPR leader sequence; and
wherein the second cell comprises
iv. a third transgene nucleic acid sequence comprising a second CRISPR direct repeat sequence (second DR sequence) and a CRISPR leader sequence; and
wherein the first and the second DR sequences differ in at least one nucleotide; and
wherein said first and second DR sequences and said CRISPR leader sequence are specifically recognizable by an RT-Cas1-Cas2 complex formed by the expression products of said first transgene nucleic acid sequence and said second transgene nucleic acid sequence.

2. The composition according to item 1, wherein the first DR sequence is SEQ ID NO 01 (GTTGTACCTTACCTATGAGGAATTGAAAC) and the second DR sequence is SEQ ID NO 02 (GTCGTACTTTACCTAAAAGGAATTGAAAC).

3. The composition according to item 1, wherein the first DR sequence is (GTTGTACCTTACCTATGAGGAATTGAAAC or GTCGTACTTTACCTAAAAGGAATTGAAAC) and the second DR sequence differs from the first DR sequence in at least one nucleotide, particularly in 4, 3, 2, or 1 nucleotide(s), more particularly in two nucleotides.

4. The composition according to any one of the preceding items, wherein the first and the second DR sequence are selected from different sequences of the group of SEQ ID NO 01 to SEQ ID NO 11.

5. The composition according to any one of the preceding items, wherein the first DR sequence and/or the second DR sequence are individually associated with a third transgene promoter in addition to the leader sequence, wherein the third transgene promoter is located in close proximity, more particularly 5′ direction, thereof.

6. The composition according to item 5, wherein said third transgene promoter is a weak promoter, particularly a weak constitutive RNA polymerase promoter.

7. The composition according to any one of the preceding items, wherein the first and the second cell are of the same species, particularly of the species E. coli.

8. The composition according to item 7, wherein the first and the second cell differ in expression of at least one gene.

9. The composition according to item 8, wherein the one gene encodes an enzyme catalyzing an essential metabolic step.

10. The composition according to any one of the preceding items, wherein the first and/or second transgene promoter sequence is an inducible promoter; particularly wherein the first and second transgene promoter sequence are one and the same promoter sequence.

11. The composition according to any one of the preceding items, wherein the first transgene nucleic acid sequence is under transcriptional control of a first promoter sequence and the second transgene nucleic acid sequence promoter is under transcriptional control of a second promoter sequence, particularly wherein each of said first and second promoter sequence is a different inducible promoter sequence.

12. The composition according to any one of the preceding items for use in diagnosis.

13. A method for monitoring of a diet of a patient or for diagnosis of a disease of a patient, particularly of a digestive or gastrointestinal disorder of a patient, said method comprising the steps of
collecting a composition of cells according to any one of the preceding items 1 to 12, or DNA from said composition of cells, from a feces sample collected from said patient, wherein said cells had been previously applied orally to said patient,
isolating the third transgene nucleic acid sequence from said cells, or amplifying said third transgene sequence from said DNA, yielding isolated third transgene nucleic acid sequences, and
sequencing said isolated third transgene nucleic acid sequences, and distinguishing said isolated third transgene nucleic acid sequences derived from the first cell from the said isolated third transgene nucleic acid sequences derived from the second cell by the difference in the first and second DR sequence;
thereby recording one or more transcripts of said composition of cells produced in the environment of the gastrointestinal tract.

14. A cell comprising
i. a first transgene nucleic acid sequence encoding a fusion protein comprising or essentially consisting of a reverse transcriptase polypeptide and a Cas1 polypeptide,
ii. a second transgene nucleic acid sequence encoding a Cas2 polypeptide, and
iii. a third transgene nucleic acid sequence comprising a CRISPR direct repeat sequence (DR sequence) and a CRISPR leader sequence;
wherein said DR sequence and said CRISPR leader sequence are specifically recognizable by an RT-Cas1-Cas2 complex formed by the expression products of said first transgene nucleic acid sequences and said second transgene nucleic acid sequence, and
wherein a third transgene promoter, particularly a weak third transgene promoter, is associated with, particularly located in 5′ direction of, the DR sequence.

15. The cell according to item 14 for use in diagnosis.

16. A method for monitoring of a diet of a patient or for diagnosis of a disease of a patient, particularly of a digestive or gastrointestinal disorder of a patient, said method comprising the steps of
collecting a cell according to item 14, or DNA from said cell, from a feces sample collected from said patient, wherein said cell had been previously applied orally to said patient,
isolating the third transgene nucleic acid sequence from said cell, yielding an isolated third transgene nucleic acid sequence, and
sequencing said isolated third transgene nucleic acid sequence.
thereby recording a transcript of said cell produced in the environment of the gastrointestinal tract.

17. A method for diagnosis of a condition affecting the intestine of a patient, said method comprising the steps:
i. collecting cells, or DNA from said cells, from a feces sample collected from the patient, wherein said cells had previously been applied orally to said patient;
wherein the cells prior to the oral application comprised:
a first transgene nucleic acid sequence encoding a fusion protein comprising or essentially consisting of a reverse transcriptase polypeptide and a Cas1 polypeptide and a second transgene nucleic acid sequence encoding a Cas2 polypeptide, said first transgene nucleic acid sequence and said second transgene nucleic acid sequence being under transcriptional control of an, optionally inducible, promoter sequence, and
a third transgene nucleic acid sequence comprising a CRISPR direct repeat sequence; said CRISPR direct repeat sequence being specifically recognizable by an RT-Cas1-Cas2 complex formed by the expression products of said first transgene nucleic acid sequence and said second transgene nucleic acid sequence,
wherein in the collected cells, spacers derived from a plurality of RNA molecules of the cell have been integrated into the CRISPR direct repeat sequence, yielding a modified third transgene nucleic acid sequence;
ii. isolating the modified third transgene nucleic acid sequence from said cells, or amplifying said modified third transgene nucleic acid sequence from said DNA, yielding an isolated modified third transgene nucleic acid sequence, and
iii. sequencing said isolated modified third transgene nucleic acid sequence;
wherein a high probability of having a condition affecting the intestine is assigned to said patient if,
when compared to a plurality of reference cells collected from a subject without said condition affecting the intestine
   or
after longitudinal studies of single patients before and following dietary or therapeutic interventions (such as correction or dietary deficiencies, prescription of diets lacking intolerance components, anti-inflammatory treatments, prokinetic administration, antibiotic usage, stool transplantation),
the modified third transgene nucleic acid sequences isolated from said patient comprise spacers derived from an indicator gene,
A) wherein the condition is selected from the list comprising:
   intestinal inflammation;
and the indicator gene is selected from the list comprising the following genes:
alaE; ipbAB; lon; mqsAR; pspABCDEGH; spy; tcdABCDEFG; tomB; adhE; dmsABCD; eda; edd; gadABCE; gntK; hdeABD; napABCDFGH; narGHIJKUWYZ; gntTU; kdgKT; nagABCEKZ; nanACEKMQRSTXY; uxaABC;
B) wherein the condition relates to the nutritional status of the patient (macronutrient insufficiency or excess; caloric adequacy after supplementation in malnutrition; micronutrient vitamin and mineral insufficiency), oxidative stress, intestinal inflammation, dysbiosis interaction between different taxa within the intestinal microbiota, niche adequacy for individual microbiota taxa, carbohydrate and lipid malabsorption (functional lactase, sucrase/isomaltase, trehalase and pancreas exocrine deficiencies), control for presence of gluten in diet of celiac disease patients, control for presence of FODMAPs (fermentable oligosaccharides, disaccharides, monosaccharides, and polyols) in diet of patients with intestinal functional disorders and bloating), bile salt malabsorption, delayed intestinal transit disorders (sclerosing conditions such as systemic sclerosis, neuromuscular disorders of the intestinal tract, intestinal changes in autonomic neuropathy of enteric neuropathies);
   particularly wherein the condition is malnutrition,
and wherein the indicator gene is selected from the list comprising the following genes: eda; edd; gatYZ; gntKPTU; idnKP; kdgKT; kduDI; nagABCEKZ; nanACEKMQRSTXY; nirB; uxaABC; uxuABC; zraP; alaE; dmsABCD; gadABC; gadABCE; hdeABD; hyaABC; lacAZ; napABCDFGH; narGIJKUWYZ.

18. The method according to item 17, wherein the condition is inflammation of the intestine, and wherein a high probability of the patient suffering from intestinal inflammation is assigned to said patient if, when compared to a reference collected from a subject without malnutrition, the isolated modified third transgene nucleic acid sequences comprise a significantly different amount of spacers derived from genes selected from the list:
alaE; acrZ; bhsA; cpxP; glgS; Hha; ibpA; ibpB; lon; mqsA; mqsR; osmB; pspA; pspB; pspC; pspD; pspE; pspG; pspH; spy; tdcA; tdcB; tdcC; tdcD; tdcE; tdcF; tdcG; tomB; adhE; dmsA; dmsB; dmsC; dmsD; eda; edd; gadA; gadB; gadC; gadE; gntK; hdeA; hdeB; hdeD; hyaA; hyaB; hyaC; napA; napB; napC; napD; napF; napG; napH; narG; narH; narl; narJ; narK; narU; narW; narY; narz; gntP; gntT; gntU; idnK; idnP; kdgK; kdgT; kduD; kdul; nagA; nagB; nagC; nagE; nagK; nagZ; nanA; nanC; nanE; nanK; nanM; nanQ; nanR; nanS; nanT; nanx; nanY; uxaA; uxaB; uxaC; uxuA; uxuB; uxuC.

19. The method according to item 17, wherein a high probability of malnutrition is assigned to said patient if the modified third transgene nucleic acid sequences isolated from said patient, compared to sequences obtained from reference cells, comprises
a significantly higher amount of a spacer derived from a gene selected from the list comprising eda; edd; gatYZ; gntKPTU; idnKP; kdgKT; kduDI; nagABCEKZ; nanACEKMQRSTXY; nirB; uxaABC; uxuABC; zraP and/or
a significantly lower amount of a spacer derived from a gene selected from the list comprising alaE; dmsABCD; gadABC; gadABCE; hdeABD; hyaABC; lacAZ; napABCDFGH; narGIJKUWYZ.

20. An isolated nucleic acid molecule comprising a direct repeat sequence selected from the group comprising SEQ ID NO 1, SEQ ID NO 02, SEQ ID NO 03, SEQ ID NO 04, SEQ ID NO 05, SEQ ID NO 06, SEQ ID NO 07, SEQ ID NO 08, SEQ ID NO 09, SEQ ID NO 10, SEQ ID NO 11.

The invention is further illustrated by the following examples and figures, from which further embodiments and advantages can be drawn. These examples are meant to illustrate the invention but not to limit its scope.

### DESCRIPTION OF THE FIGURES

FIG. 1 shows transcriptional recording sentinel cells acquire transcriptional records within the mouse gut and preserve this information throughout time. (A) Schematic of experimental workflow for in vivo experiments with transcriptional recording sentinel cells. E. coli with recording plasmid encoding anhydrotetracycline (aTc)-inducible FsRT-Cas1-Cas2 and a CRISPR array were orally gavaged into mice. Record-seq was performed on feces or intestinal contents. (B and C) Numbers of E. coli genome-aligning spacers (B) obtained from feces on the indicated days and (C) from the indicated intestinal sections on day 20 after gavage. B and C show mean±s.e.m. of n=5 independent biological replicates. (D) Timeline of longitudinal in vivo recording experiment assessing the impact of diet on the intestinal E. coli transcriptome. Germ-free mice were supplied with aTc in the drinking water and gavaged with E. coli MG1655 transformed with the recording plasmid. Mice were fed a chow, fat, or starch diet starting 2 days prior to gavage until day 7. After day 7, all groups received the chow diet. Fecal RNA/Record-seq sampling is indicated, with day 7 samples being collected prior to changing the diets. Data for days 1 to 6, 8, 10, 11, 13, and 16 are shown in FIG. 1B, FIGS. 7A and 8C. (E and F) UMAP-based visualization of (E) RNA-seq or (F) Record-seq data from E. coli under chow (blue), fat (orange), or starch (green) diet on indicated days corresponding to FIG. 1D. The dot sizes denote successive time points; n=5 independent biological replicates. Count thresholds were 10⁴ (Record-seq) and 10⁵ (RNA-seq). Outliers were excluded based on modified Z-score and relative deviation from the mean (see methods).

FIG. 2 shows record-seq reveals transcriptional changes describing the adaptation of E. coli to diet-dependent intraluminal environments. (A to C) Record-seq data from day 7 feces of mice fed a chow (blue), fat (orange), or starch (green) diet. (A) Heatmap showing hierarchical clustering using 1183 differentially expressed genes (DEG). Z-score standardized gene-aligning spacer counts are shown. (B) Volcano plot with DEGs (P_{adj}<0.1, log₂ fold change>1.5) indicated. (C) Pathways and transcriptional/translational regulators enriched per diet group using EcoCyc. Dot sizes show gene numbers detected as significantly upregulated for the respective pathway. (D) Box plot showing E. coli genome-aligning spacer counts for selected genes involved in gluconate metabolism corresponding to the indicated diets (E) Competitive colonization experiment. Germ-free mice fed either chow or starch diet were orally gavaged with a 1:1 mixture of wild-type (wt) E. coli MG1655 and E. coli MG1655 ΔgntK/ΔidnK (mut). Competitive indices were calculated from fecal recoveries as the ratio of mutant to wt CFU (mean±s.e.m., n=5 independent biological replicates, P=3.93.10⁻¹⁷ Likelihood-ratio test (representative result of two independent experiments). Panels A to D correspond to FIG. 1D, n=5 independent biological replicates. Count thresholds were 10⁴ (Record-seq) and 10⁵ (RNA-seq). Outliers were excluded based on modified Z-score and relative deviation from the mean (see methods).

FIG. 3 shows record-seq sentinel cells capture the milieu along the length of the intestine and preserve transient features of proximal large intestinal segments. Mice colonized with Record-seq sentinel cells were fed a chow or starch diet for 7 days. (A) Stacked bar plots showing fractions of Record-seq differentially expressed genes (DEGs) that were also detected by RNA-seq from feces or intestinal segments as upregulated under a chow (430 genes) or starch diet (278 genes). Record-seq DEGs not identified by fecal RNA-seq (left bar) are categorized (right bar) based on differential RNA expression in isolated gut sections: cecum or proximal colon (“proximal”), distal colon (“distal”), or both proximal and distal sections (“distal & proximal”). (B) Heatmap of cecum signature genes (213 genes overexpressed in the cecum) showing hierarchical clustering of rank-normalized RNA-seq and Record-seq data from the indicated intestinal sections from mice fed a chow diet. Data in (A) and (B) are a combined analysis of n=3 biological replicates per group each pooled from n=3 individual mice for gut sections RNA-seq and n=5 biological replicates per group for fecal RNA-seq and Record-seq. Count thresholds were 10⁴ (Record-seq) and 10⁵ (RNA-seq). Outliers were excluded based on modified Z-score and relative deviation from the mean (see methods). (C) Competitive colonization experiment. Germ-free mice fed either a chow or starch diet were orally gavaged with a 1:1 mixture of wild-type (wt) E. coli MG1655 and E. coli MG1655 ΔuxaC. Competitive indices were calculated from fecal ratios of mutant to wt CFU (mean±s.e.m., n=5 independent biological replicates, P=0.0022 Likelihood-ratio test (representative result of two independent experiments)).

FIG. 4 shows record-seq provides a non-invasive assessment of DSS-induced intestinal inflammation. (A) Timeline of DSS colitis recording experiment. Germ-free mice were supplied with aTc in the drinking water, gavaged with E. coli BL21 (DE3) sentinel cells and received 1, 2, or 3% DSS or water as indicated. Fecal Record-seq sampling is indicated. (B) UMAP-based visualization of Record-seq data for control mice (blue) or mice treated with 1% (salmon), 2% (red), and 3% (black) DSS. Dot sizes denote successive time points. Mice receiving 3% DSS had to be euthanized on day 13. (C) Heatmap showing hierarchical clustering of Record-seq differentially expressed genes (DEGs) from control mice (blue) or mice treated with 1% (salmon) and 2% (red) DSS, day 19. Z-score standardized gene-aligning spacer counts are shown. (D) Timeline of DSS colitis recording experiment. Germ-free mice were supplied with aTc in the drinking water, gavaged with E. coli MG1655 sentinel cells and given 2% DSS or water as indicated. Fecal Record-seq sampling is indicated. (E) PCA-based visualization of Record-seq data on days 7, 8, 10, 14, 17, and 20 for control mice (blue) or mice treated with 2% DSS (red). K-medoids clusters are indicated by convex hulls. Dot size denote successive time points. (F) Dot plot showing log₂-fold changes for Record-seq DEGs identified for control mice (blue) or mice treated with 2% DSS (red). Dot size increases with significance (P_{adj}: 9.8·10⁻¹⁹-1.0). (G and H) STRING analysis of DEGs significantly up-(G) or downregulated (H) under DSS treatment compared to control. Node size increases with log₂-fold change (G: 0.5-4.7, H: 0.5-2.9). Panels B and C correspond to the experiment in FIG. 4A with n=3 independent biological replicates. Panels E to H correspond to the experiment in FIG. 4D with n=3-4 independent biological replicates of each condition. Count thresholds were 5·10³ (B and C) and 10⁴ (E to H). Outliers were excluded based on modified Z-score and relative deviation from the mean (see methods).

FIG. 5 shows record-seq illuminates both host-microbe and microbe-microbe interactions. (A) Timeline of longitudinal in vivo recording experiment on interaction of E. coli with Bacteroides thetaiotaomicron (B. theta) in the gut. Germ-free mice were supplied with aTc in the drinking water and gavaged with E. coli MG1655 sentinel cells alone or together with B. theta. Fecal Record-seq sampling is indicated. (B) UMAP-based visualization of Record-seq data from E. coli in the presence (yellow) or absence (blue) of B. theta on the indicated days. Dot size denotes successive time points. (C) Heatmap showing hierarchical clustering of Record-seq data from E. coli in the presence (yellow) or absence (blue) of B. theta on the indicated days using identified differentially expressed genes. Z-score standardized gene-aligning spacer counts are shown. (D) Pathways and transcriptional/translational regulators identified as enriched (P<0.05) by EcoCyc in E. coli in the presence (yellow) or absence (blue) of B. theta on days 2 to 27. Dot sizes represent gene numbers significantly upregulated for the respective pathway. (E) Schematic depicting nutrient cross-feeding relationship between E. coli and B. theta inferred by Record-seq. E. coli genes encoding transporters and enzymes are depicted with color-codes reflecting their Record-seq-based log₂-fold upregulation in the presence versus absence of B. theta (0-5.0). Panels B to D correspond to FIG. 5A with n=4 independent biological replicates of each condition. Count threshold was 5·10³. Outliers were excluded based on modified Z-score and relative deviation from the mean (see methods).

FIG. 6 shows sentinel cells are deployable within a complex microbiota. (A) Timeline of complex microbiota recording experiment. Mice harboring a representative 12-member intestinal microbiota (sDMDMm2) were placed on either a chow or starch diet, supplied with aTc in the drinking water, and gavaged with a dose of 7·10¹⁰ CFU aTc-pretreated E. coli MG1655 as indicated. Fecal Record-seq sampling is indicated. (B) Dot plot showing the log₂-fold change for Record-seq differentially expressed genes (DEGs) corresponding to the indicated diet and time. Dot size increases with significance (P_{adj}: 1.9·10⁻¹⁷-1.0). Colored dots indicate a P_{adj}<0.1, gray dots indicate non-significant differences. (C) Heatmap showing hierarchical clustering of Record-seq data at 21 hours from mice fed a chow (blue) or starch (green) diet based on identified DEGs. Z-score standardized gene-aligning spacer counts are shown. (D) Pathways and transcriptional/translational regulators identified as enriched (P<0.05) in mice fed chow (blue) or starch (green) using EcoCyc. Dot size increases with number of genes detected as significantly upregulated for the respective pathway. (E) Schematic of experimental workflow for performing multiplexed in vivo intestinal recording experiments with transcriptional recording sentinel cells barcoded by their CRISPR array. Enhanced efficiency recording plasmids (FIG. 15A) encoding FsRT-Cas1-Cas2 under transcriptional control of an anhydrotetracycline (aTc)-inducible promoter and either leader-DR1 or leader-DR2 downstream of the constitutive promoter BBa_J23103 are transformed into wild type E. coli or mutant E. coli cells and orally gavaged into mice in an equal mixture. Multiplexed Record-seq performed on the mixed sentinel cells (Methods) reveals host-microbial mutualism and strain-specific transcriptional archives from two isogenic strains of E. coli present in the same mouse. (F) Heatmap showing hierarchical clustering of Record-seq data from a longitudinal multiplexed recording experiment with a full factorial design between the CRISPR array-barcoded recording plasmid and the bacterial strain. Group 1 was given an inoculum consisting of an equal mixture of ΔuxaC or wild type E. coli harboring a leader-DR1—(‘ΔuxaC-DR1’, blue) or leader-DR2—(‘wt-DR2’, pink) barcoded recording plasmid, respectively. Group 2 was given an inoculum where the pairing between CRISPR array-barcoded plasmids and bacterial strains were swapped, namely an inoculum consisting of an equal mixture of ΔuxaC or wild type E. coli harboring a leader-DR2—(‘ΔuxaC-DR2’, green) or leader-DR1—(‘wt-DR1’, orange) barcoded recording plasmid, respectively. Hierarchical clustering was performed using 50 high confidence differentially expressed genes. Z-score standardized gene-aligning spacer counts are shown as expression values. (G) Pathways and transcriptional/translational regulators identified as enriched (p<0.05) using EcoCyc based on identified high confidence differentially expressed genes between ΔuxaC (red) wild type (blue) E. coli using Record-seq data. The size of the dots increases with number of genes detected as significantly upregulated for the respective pathway. Panels B to D correspond to FIG. 6A with n=5 independent biological replicates. Panel F and G correspond to FIG. 15H with n=5 independent biological replicates.

FIG. 7 shows transcriptional recording sentinel cells acquire transcriptional records within the mouse gut and preserve this information throughout time. (A and B) Bar plots showing the cell number used per Record-seq input as estimated by droplet digital PCR (ddPCR) from (A) feces on the indicated days after gavage of E. coli sentinel cells and (B) different gut sections on day 20. The concentration of pFS_0453 was measured by ddPCR and the number of cells was calculated assuming 20 copies of pFS_0453 (pET30b+ origin of replication) per E. coli cell. Shown is the mean±s.e.m. of n=5 independent biological replicates. (C) Bar plot showing the number of E. coli genome-aligning spacers obtained from colon or cecum contents of mice supplied with various concentrations of anhydrotetracycline (aTc) in the drinking water. Shown is the mean of n=2-4 independent biological replicates. (D) Bar plot showing the number of E. coli genome-aligning spacers and recording plasmid-derived spacers. Shown is the mean±s.e.m. of n=20 independent biological replicates of chow-fed mice corresponding to a total of 3,249,165 spacers (3,123,056 genome-aligning, 126,109 plasmid-aligning). (E) Bar plot showing the percentage of spacers aligning to the sense or antisense strand of E. coli genes. (F and G) Histograms showing the (F) length and (G) GC content distribution of E. coli genome-aligning spacers.

FIG. 8 shows Record-seq reveals transcriptional changes describing the adaptation of E. coli to diet-dependent intraluminal environments. (A and B) PCA-based visualization of RNA-seq (A) and Record-seq (B) data from mice fed a chow (blue), fat (orange), or starch (green) diet on day 7. (C) UMAP-based visualization of Record-seq data from mice fed a chow (blue), fat (orange), or starch (green) diet on days 2 to 20. Dot sizes denote successive time points. (D and E) Heatmap showing hierarchical clustering of (D) RNA-seq and (E) Record-seq data on day 20 using top 500 diet-specific signature genes identified prior to the diet switch on day 7. Z-score standardized gene-aligning spacer counts are shown. Panels A to E correspond to FIG. 1D with n=5 independent biological replicates for each diet. Count thresholds were 10⁴ (Record-seq) and 10⁵ (RNA-seq). Outliers were excluded based on modified Z-score and relative deviation from the mean (see methods).

FIG. 9 shows record-seq results are reproducible across independent experiments. (A) Timeline of longitudinal in vivo recording experiment assessing the impact of diet on the E. coli transcriptome inside the gut. This was an independent replicate of the experiment in FIG. 1D. Germ-free mice were supplied with aTc in the drinking water and orally gavaged with E. coli sentinel cells. Mice were fed a chow, fat, or starch diet 2 days prior to gavage until day 7 of the experiment. From day 7 onwards, all groups received a chow diet. Fecal sampling for Record-seq and/or RNA-seq is indicated. (B and C) PCA-based visualization of (B) RNA-seq or (C) Record-seq data on day 7. (D and E) PCA-based visualization of (D) RNA-seq or (E) Record-seq data on day 14. (F) UMAP-based visualization of Record-seq data on days 2 to 14 from mice fed chow (blue), fat (orange), or starch (green) diet until day 7. Dot sizes denote successive time points. (G and H) Heatmap showing hierarchical clustering of (G) RNA-seq or (H) Record-seq data on day 14 using top 500 diet-specific signature genes identified prior to the diet switch on day 7. Z-score standardized gene-aligning spacer counts are shown. (I) Scatter plot showing the correlation in log₂-fold change of DEGs and percentage of these genes regulated in the same direction for the two diet experiments outlined in FIG. 1D and FIG. 9A. Genes detected as differentially expressed in Record-seq in chow versus starch groups on day 7 in the diet experiment outlined in FIG. 1D were used to perform this analysis. Panels B to H correspond to FIG. 9A with n=5 independent biological replicates for each condition. Count thresholds were 10⁴ (Record-seq) and 10⁵ (RNA-seq). Outliers were excluded based on modified Z-score and relative deviation from the mean (see methods).

FIG. 10 shows record-seq reveals transcriptional changes describing E. coli's adaptation to diet-dependent intraluminal environments. (A and B) Volcano plots showing Record-seq differentially expressed genes (DEGs) from mice fed a (A) chow (blue) or fat (orange) diet or a (B) fat (orange) or starch (green) diet on day 7 as shown in FIG. 1D scheme (P_{adj}<0.1; log₂-fold change>1.5). (C and D) Pathways and transcriptional/translational regulators identified as enriched (P<0.05) using EcoCyc based on Record-seq data on day 7 from mice fed a (C) chow (blue) or fat (orange) diet or a (D) fat (orange) or starch (green) diet. Dot sizes show gene numbers detected as significantly upregulated for the respective pathway. (E and F) STRING analysis of genes significantly upregulated in E. coli from mice fed a (E) chow or (F) starch diet. Node size corresponds to log₂-fold change of upregulation (E: 1.0-5.0, F: 1.0-4.4). Panels A-F correspond to FIG. 1D with n=5 independent biological replicates.

FIG. 11 shows record-seq sentinel cells capture the milieu of proximal gut sections in a non-invasive fashion. (A and B) PCA-based visualization of E. coli RNA-seq data from cecum (green), proximal colon (orange), and distal colon (purple) of mice fed a (A) chow or (B) starch diet on day 7. (C and D) PCA-based visualization of E. coli RNA-seq data from cecum (green), proximal colon (orange), distal colon (purple), and feces (pink) of mice fed a (C) chow or (D) starch diet. (E) Heatmap of cecum signature genes (213 genes overexpressed in the cecum) showing hierarchical clustering of rank-normalized RNA-seq and Record-seq data from the indicated intestinal sections from mice fed a starch diet. (F) Box plot showing E. coli rank-normalized counts of uxaC as determined by Record-seq or RNA-seq from feces, cecum, proximal colon and distal colon corresponding to the indicated diets on day 7. (G) Heatmap showing log₂-fold change as determined by Record-seq or RNA-seq from feces, cecum, proximal colon and distal colon for genes that were experimentally validated (uxaAC in FIG. 3C, gadABC, hdeAB in FIG. 11H) as a subset of genes identified as differentially regulated in the chow and starch diet groups by fecal Record-seq but not fecal RNA-seq. Grey boxes indicate no significant differential regulation. (H) Box plot showing cecal pH under chow or starch diet. Representative result from two independent experiments of n=5 mice per group. P=1.975·10⁻⁵ (T-test). Panels A and B with n=3 independent biological replicates each pooled from n=3 individual mice. Panels C and D with n=5 independent biological replicates each from an individual mouse. Panel E is a combined analysis from n=3 independent biological replicates each pooled from n=3 individual mice. Count thresholds were 10⁴ (Record-seq) and 10⁵ (RNA-seq). Outliers were excluded based on modified Z-score and relative deviation from the mean (see methods). Panels A and B with n=3 independent biological replicates each pooled from n=3 individual mice. Panels C and D with n=5 independent biological replicates each from an individual mouse. Panel E is a combined analysis from n=5 independent biological replicates.

FIG. 12 shows record-seq provides a non-invasive assessment of DSS-induced colitis. (A) PCA-based visualization of Record-seq data of E. coli exposed in vitro to 0 (blue), 0.1% (light pink), 0.3% (salmon), 1% (red), or 3% (black) dextran sulfate sodium (DSS), n=5 independent biological replicates. (B and C) Box plots showing (B) fecal lipocalin levels and (C) percent of initial weight on day 10 in control mice (blue) or mice treated with 1% (salmon), 2% (red), or 3% (black) DSS, n=3 for each condition (D) PCA-based trajectory plot of Record-seq data from control mice (blue) and mice treated with 1% (salmon), 2% (red), or 3% (black) DSS. Convex hulls represent k-medoids clusters. Dot sizes denote successive time points. (E) Area under the receiver operating characteristic curve (AUCROC) for evaluating the performance of multi-class SVM classifiers for distinguishing Record-seq samples based on DSS treatment groups. (F) Line plot showing fecal lipocalin levels from control mice (blue) or mice treated with 2% DSS (red), shown is mean±s.e.m. (G) UMAP-based visualization of Record-seq data from control mice (blue) or mice treated with 2% DSS (red), days 2 to 20. Dot sizes denote successive time points. (H) Heatmap showing hierarchical clustering of Record-seq data from control mice (blue) or mice treated with 2% DSS (red), using differentially expressed genes identified on day 20. Z-score standardized gene-aligning spacer counts are shown. (I) Pathways and transcriptional/translational regulators identified as enriched (P<0.05) using EcoCyc based on Record-seq data on days 2-20 for control mice (blue) or mice treated with 2% DSS (red). Dot size increases with number of significantly upregulated genes for the respective pathway. Panels B to E correspond to FIG. 4A with n=3 independent biological replicates. Panels F to I correspond to FIG. 4D with n=3-4 independent biological replicates. Count thresholds were 10⁴ (panels A and G to I). 5·10³ (panels D and E). Outliers were excluded based on modified Z-score and relative deviation from the mean (see methods). Panels B to E corresponds to FIG. 4A with n=3 independent biological replicates. Panels F to I correspond to FIG. 4D with n=3-4 independent biological replicates.

FIG. 13 shows record-seq illuminates both host-microbe and microbe-microbe interactions. (A and B) STRING analysis of genes significantly (A) upregulated or (B) downregulated in E. coli in the presence of B. theta compared to E. coli in monocolonized mice. Node size indicates log₂-fold change (panel A: 0.2-5.7, panel B: 0.2-6.4). (C) Bar plot showing E. coli colony forming unit (CFU) counts per g of feces on day 7, shown is mean±s.e.m., P=0.02857 (Wilcoxon rank sum test). (D) Timeline of longitudinal in vivo recording experiment for illuminating the interaction of E. coli with B. theta in the mouse gut. Germ-free mice were supplied with aTc in the drinking water and orally gavaged with E. coli sentinel cells alone or together with B. theta. Fecal Record-seq sampling is indicated. (E) UMAP-based visualization of Record-seq data from E. coli in the presence (yellow) or absence (blue) of B. theta on days 4 to 9. Dot sizes denote successive time points. (F) Heatmap showing hierarchical clustering of Record-seq data from E. coli in the presence (yellow) or absence (blue) of B. theta on indicated days using identified differentially expressed genes (DEGs). Z-score standardized gene-aligning spacer counts are shown. (G) Scatter plot showing the correlation in log₂-fold change of DEGs and percentage of these genes regulated in the same direction for the two experiments outlined in FIG. 5A and FIG. 13D. Genes detected as differentially expressed for Record-seq from E. coli in the presence (yellow) or absence (blue) of B. theta in the experiment outlined in FIG. 5A were used to perform this analysis. Panels A to C correspond to the experiment outlined in FIG. 5A with n=4 independent biological replicates. Panels E and F correspond to the experiment outlined in FIG. 13D with n=4-5 independent biological replicates. Count threshold was 5·10³. Outliers were excluded based on modified Z-score and relative deviation from the mean (see methods). Panels A to C correspond to the experiment outlined in FIG. 5A with n=4 independent biological replicates. Panels E and F correspond to the experiment outlined in Fig. S7D with n=4-5 independent biological replicates.

FIG. 14 shows sentinel cells are deployable within a complex microbiota. (A) Bar plot showing E. coli colony forming unit (CFU) counts per g of feces at the indicated timepoints. (B) Bar plot showing the number of E. coli genome-aligning spacers obtained per Record-seq sample from the feces of sDMDMm2 mice at indicated timepoints after gavage of 7·10¹⁰ E. coli sentinel cells corresponding to FIG. 6A. Shown is the mean±s.e.m. (C) UMAP-based visualization of Record-seq data from mice fed a chow (blue), or starch (green) diet at 6, 10, 14, 18, and 21 hours. Dot sizes denote successive points. (D) Scatter plot showing the correlation in log₂-fold change of DEGs and percentage of these genes regulated in the same direction for the two experiments outlined in FIG. 6A and an independent replicate experiment using a gavage dose of 6·10¹⁰ E. coli sentinel cells. Genes detected as differentially expressed for Record-seq from E. coli in the in sDMDMm2 mice on the chow or starch diet in the experiment outlined in FIG. 6A were used to perform this analysis. (E) Heatmap showing hierarchical clustering of Record-seq data at 21 hours using identified differentially expressed genes (DEGs). Z-score standardized gene-aligning spacer counts are shown. (F and G) STRING analysis of genes significantly upregulated in E. coli under a (F) chow or (G) starch diet in sDMDMm2 mice. Node size indicates log₂-fold change (F: 0.4-3.2, G: 0.3-5.7). Panels A to G correspond to the experiment outlined in FIG. 6A, n=6 independent biological replicates. Panel E additionally uses data from an independent experiment with n=5 biological replicates. Count threshold was 5·10³. Outliers were excluded based on modified Z-score and relative deviation from the mean (see methods). Panels A to G correspond to the experiment outlined in FIG. 6A, shown is mean±s.e.m. of n=6 independent biological replicates. Panel E additionally uses data from an independent experiment with n=5 biological replicates.

FIG. 15 shows active transcription of the CRISPR array improves spacer acquisition. (A) Schematic illustrating: (top) the genomic CRISPR locus of Fusicatenibacter saccharivorans (Fs), which encodes two CRISPR arrays (CRISPR array 1 and CRISPR array 2) with different leader and direct repeat (DR) sequences; (middle) the first generation recording plasmid encoding FsRT-Cas1-Cas2 under transcriptional control of an anhydrotetracycline (aTc)-inducible promoter and a single terminator upstream of leader-DR2; and (bottom) the transcription-stimulated recording plasmid construct design. A double terminator downstream of FsRT-Cas1-Cas2 minimizes transcriptional readthrough from the P_{TetA} promoter whereas a constitutive promoter upstream of the leader-DR1 or leader-DR2 results in active transcription of the CRISPR array at a strength depending on the chosen promoter. (B) Bar plot showing the number of E. coli genome-aligning spacers obtained per Record-seq in vitro sample from E. coli cells transformed with the indicated transcriptional recording plasmids employing constitutive E. coli promoters from the Anderson promoter library upstream of the CRISPR array (FIG. 15A and methods) for DR1 and DR2. (C) Bar plot showing the number of E. coli genome-aligning spacers obtained per Record-seq in vivo sample from E. coli cells transformed with the indicated transcriptional recording plasmids. Panel B corresponds to an in vitro experiment with n=4 independent biological replicates. Panel C corresponds to an in vivo experiment with n=4 independent biological replicates. (D) Bar plot showing the number of reads correctly or erroneously assigned to the DR based on the library barcode (LBC) attached during the adapter ligation procedure in SENECA. Shown is the mean±s.e.m., n=12 independent biological replicates. (E) Scatter plot showing the correlation between mean normalized gene-aligning spacer-counts for Record-seq in vitro samples from E. coli cells transformed with transcriptional recording plasmid encoding FsLeader1-DR1 (pFS_1142) or FsLeader2-DR2 (pFS_1113) from hour 12. Shown is the mean of n=12 independent biological replicates. (F) Bar plot showing the number of E. coli genome-aligning spacers obtained per Record-seq in vitro sample from E. coli cells transformed with transcriptional recording plasmid encoding FsLeader1-DR1 (pFS_1142) or FsLeader2-DR2 (pFS_1113). Shown is the mean±s.e.m. of 12 independent biological replicates. Samples from the 12 and 24-hour timepoint are matched (two timepoints obtained from the same culture). (F) PCA-based visualization of Record-seq in vitro data from E. coli cells transformed with transcriptional recording plasmid encoding FsLeader1-DR1 (pFS_1142) or FsLeader2-DR2 (pFS_1113) from hour 12 and 24. The size of the dots represents sampling at progressively later times in the experiment. Shown are n=12 independent biological replicates. (G) Schematic illustrating full factorial design for multiplexed recording experiment with two experimental groups. Either wildtype E. coli transformed with leader-DR2 recording plasmid (blue) and ΔuxaC E. coli transformed with leader-DR1 recording plasmid (pink) are mixed (group 1) or wildtype E. coli transformed with leader-DR1 recording plasmid (green) and ΔuxaC E. coli transformed with leader-DR2 recording plasmid (green) are mixed and gavaged into germ-free C57BL/6. (I) Bar plot showing the number of E. coli genome-aligning spacers obtained per Record-seq sample from the feces on the indicated days after gavage from experimental group 1-gavaged with 2.9×10⁹ CFU ΔuxaC E. coli harboring DR1 recording plasmid (blue) and 4.2×10⁹ CFU wild type E. coli cells harboring DR2 recording plasmid (pink) in the same mouse, or experimental group 2-gavaged with 2.8×10⁹ of ΔuxaC E. coli harboring DR2 recording plasmid (green) and 4.5×10⁹ wild type E. coli cells harboring DR1 recording plasmid (orange) in the same mouse. (J) Heatmap showing hierarchical clustering of Record-seq data from experimental group 1 consisting of ΔuxaC E. coli harboring DR1 recording plasmid (blue) in the presence of wildtype E. coli harboring DR2 recording plasmid (pink) in the same mouse, using identified differentially expressed genes identified from days 7 to 10. Z-score standardized gene-aligning spacer counts are shown as expression values. (K) Heatmap showing hierarchical clustering of Record-seq data from experimental group 2 consisting of ΔuxaC E. coli harboring DR2 recording plasmid (green) in the presence of wildtype E. coli harboring DR1 recording plasmid (orange) in the same mouse, using identified differentially expressed genes identified from days 7 to 10. Z-score standardized gene-aligning spacer counts are shown as expression values. (L) Heatmap showing hierarchical clustering of Record-seq data from experimental group 1 consisting of ΔuxaC E. coli harboring DR1 recording plasmid (blue) in the presence of wildtype E. coli harboring DR2 recording plasmid (pink) in the same mouse or experimental group 2 consisting of ΔuxaC E. coli harboring DR2 recording plasmid (green) in the presence of wildtype E. coli harboring DR1 recording plasmid (orange) in the same mouse. The top 25 high confidence differentially expressed genes are shown. Z-score standardized gene-aligning spacer counts are shown as expression values. Panel B corresponds to an in vivo experiment with n=4 independent biological replicates. Panels D to G correspond to an in vitro experiment with n=12 independent biological replicates. Panels H to L correspond to the experiment outlined in FIG. 6E with n=5 independent biological replicates.

FIG. 16 shows (A) adapted SENECA protocol with prePCR step. (B) show GC Frequency percentage. (C) shows spacer length frequency percentage. (D) shows genome-aligning spacer counts. (E) shows genome-aligning counts of MG1655 pFS453, EcN pFS453 and EcN flu::AAC1 over time. (F) Gavaging experiments of EcN flu::AAC at 1e9 CFU/mice with collection of fecal samples at 6, 12 and 24 hours. (G) in-vitro recording qSENECA experiments.

FIG. 17 shows (A) timeline of complex microbiota recording experiment. Mice harboring a representative 12-member intestinal microbiota (sDMDMm2) were placed on either a chow or starch diet, supplied with aTc in the drinking water, and gavaged with a dose of 9.4×10E+10 CFU aTc-pretreated E. coli MG1655 as indicated. Fecal Record-seq sampling is indicated (12 h, 15 h, 18 h, 21 h, 24 h). (B) E. coli colonization levels for MG1655 transformed with pFS_1113 upon intraduodenal gavage at 9.4×10E+10 CFU/g feces at 12 h, 15 h, 18 h, and 21 h post gavage for mice on the standard rodent chow or the starch-based purified diet. Shown are mean±/−SEM for n=3-7 mice per group and time point. (C) Bar plot showing the number of E. coli genome-aligning spacers obtained per Record-seq sample from the feces of sDMDMm2 mice at the indicated timepoints after gavage of 9.4×10E+10 E. coli sentinel cells. Shown is the mean±s.e.m. (D) UMAP embedding of Record-seq data from mice fed a chow (blue), or starch (green) diet at 12, 15, 18, 18, 21 and 24 hours. Dot sizes denote successive points. (E) Dot plot showing the log₂-FC for Record-seq differentially expressed genes (DEGs) corresponding to the indicated diet and time. Dot size increases with. Colored dots indicate a P_{adj}<0.1, gray dots indicate non-significant differences. (F) Heatmap showing hierarchical clustering of Record-seq data at indicated time points from mice fed a chow (blue) or starch (green) diet based on identified DEGs. Z-score standardized gene-aligning spacer counts are shown.

### EXAMPLES

### Example 1: Transcriptional Recording Sentinel Cells Acquire Transcriptional Records within the Mouse Gut

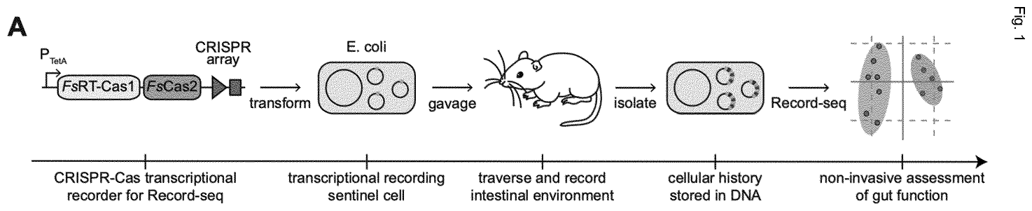

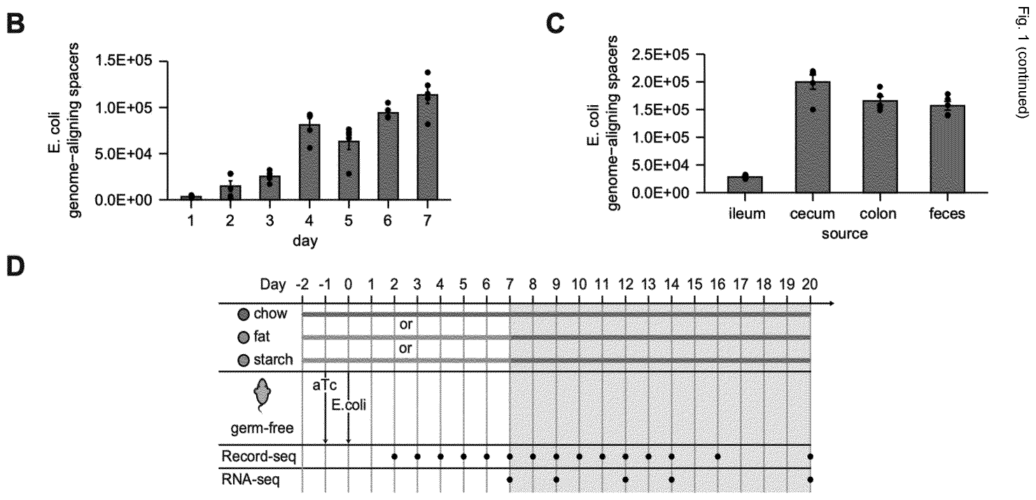

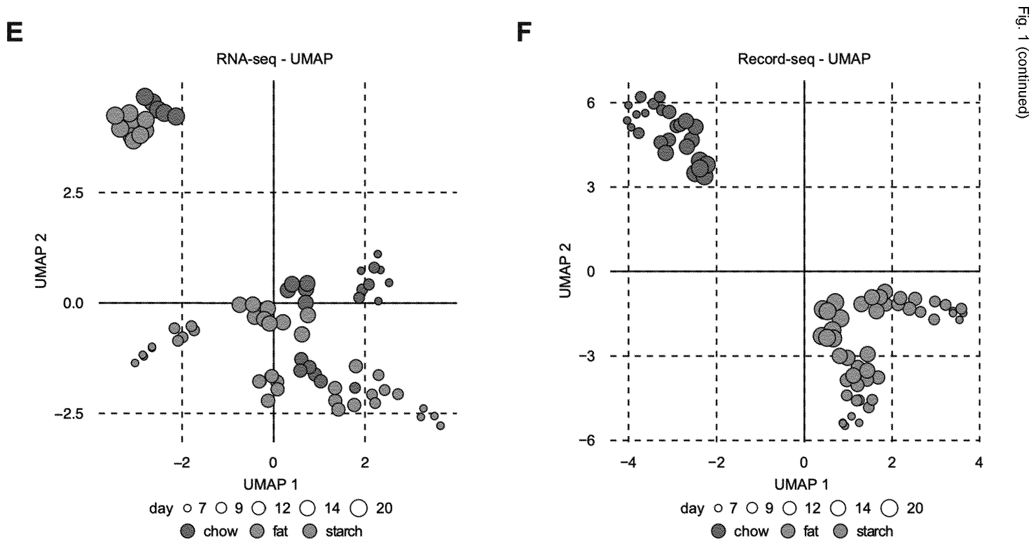

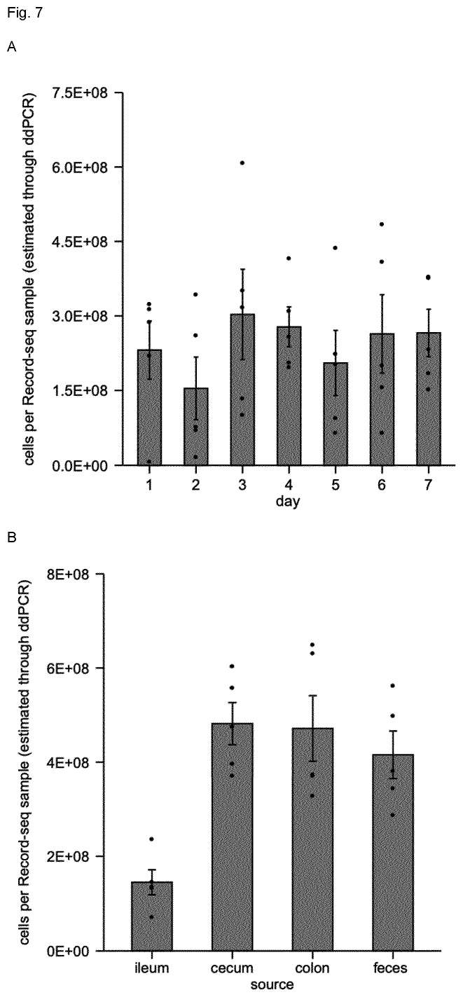

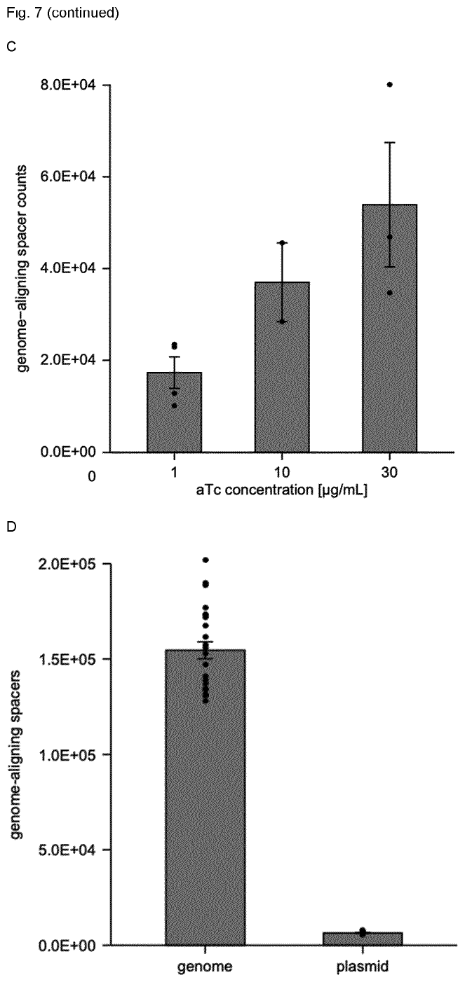

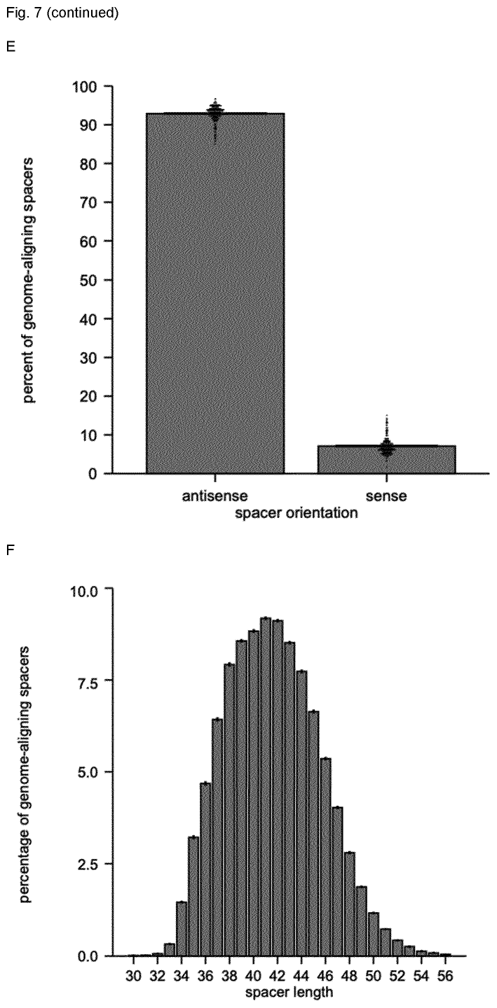

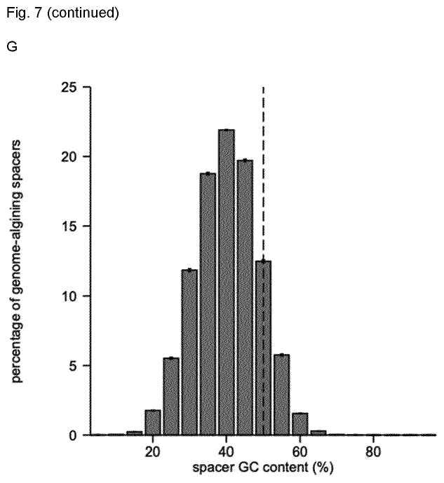

To establish transcriptional recording as a non-invasive recording tool in the mouse intestine, the inventors orally gavaged germ-free C57BL/6 (J) mice with Escherichia coli (E. coli) MG1655 carrying an anhydrotetracycline (aTc)-inducible transcriptional recording plasmid (FIG. 1A). A saturating gavage dose of 1·10⁹ colony forming units (CFU) of E. coli was used to avoid confounding the biological signal reported by the inventor's sentinel cells with an initial expansion in the gastrointestinal tract. To maintain the recording plasmid and ensure functional stability of the sentinel cells, the inventors added kanamycin sulfate to the drinking water and confirmed that the transformed cells colonized at 7.9±2.6·10⁹, comparably to what has been reported for the parental strain. Comparisons between Record-seq on sequentially-collected fecal samples and contents of the ileum, cecum and colon revealed that sentinel cells acquired increasing transcriptional records (i.e., E. coli genome-aligning spacers) throughout time and position along the intestinal tract (FIG. 1, B and C, and FIG. 7, A and B) and according to aTc concentration (FIG. 7C). The functional characteristics of FsRT-Cas1-Cas2 were consistent with the inventor's previous in vitro experiments (F. Schmidt et al., Nature 562, 380-385 (2018); T. Tanna et al., Nature protocols 15, 513-539 (2020)) (FIG. 7, D to G and Methods).

As Record-seq is a population-based measurement requiring many cells to reconstruct a cellular history, and in vivo experiments present a material-limiting environment, the inventors addressed the technical inputs and outputs of the inventor's workflow (FIG. 7, A and B). For Record-seq input, the inventors used 4.7±0.7×10⁸ cells per sample (3-4 fecal pellets of ˜27 mg each per mouse with a biomass of 5.6×10⁹ sentinel cells per gram). After 24 hours of in vivo recording, the Record-seq output was 3.6±0.6×10³ spacers, which increased by 1.0±0.5×10⁴ spacers per day. After 7 days of recording, the inventors found 1.2±0.1×10⁵ spacers, which aligned to 91±1% of the 4419 transcripts in the E. coli transcriptome. These data show that transcriptional recording sentinel cells function in vivo and accumulate transcriptome-scale records throughout time and transit of the mammalian intestine.

### Example 2: Transcriptome-Scale Recording of Complex and Dynamic Intraluminal Environments

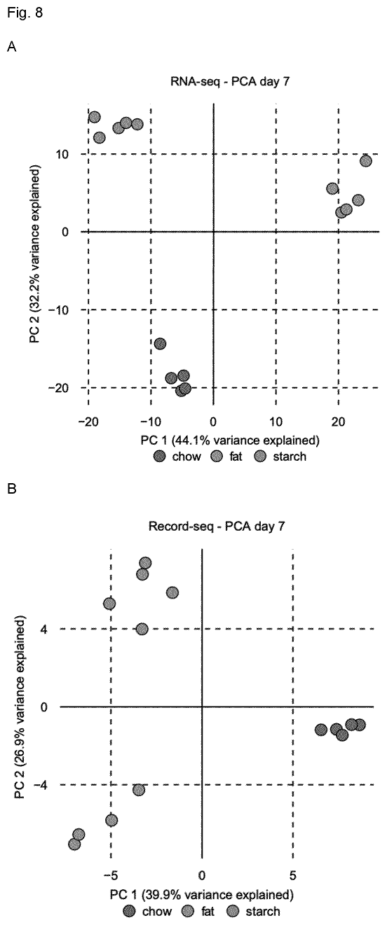

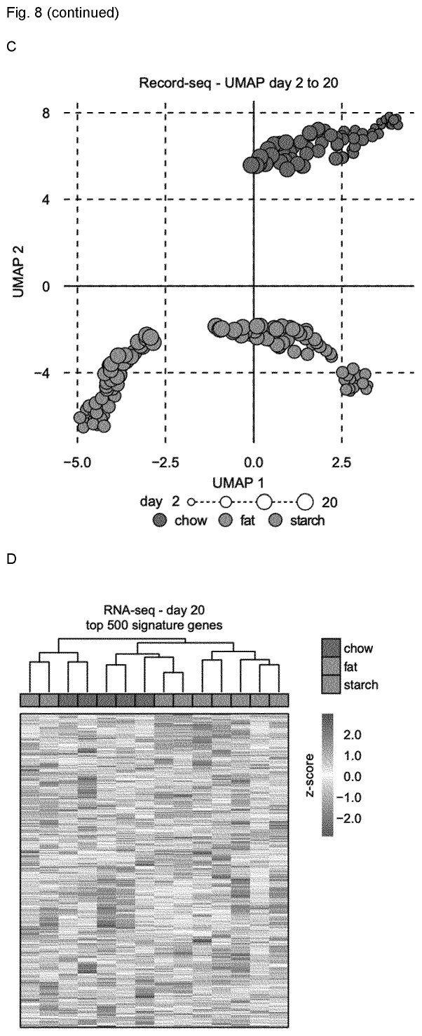

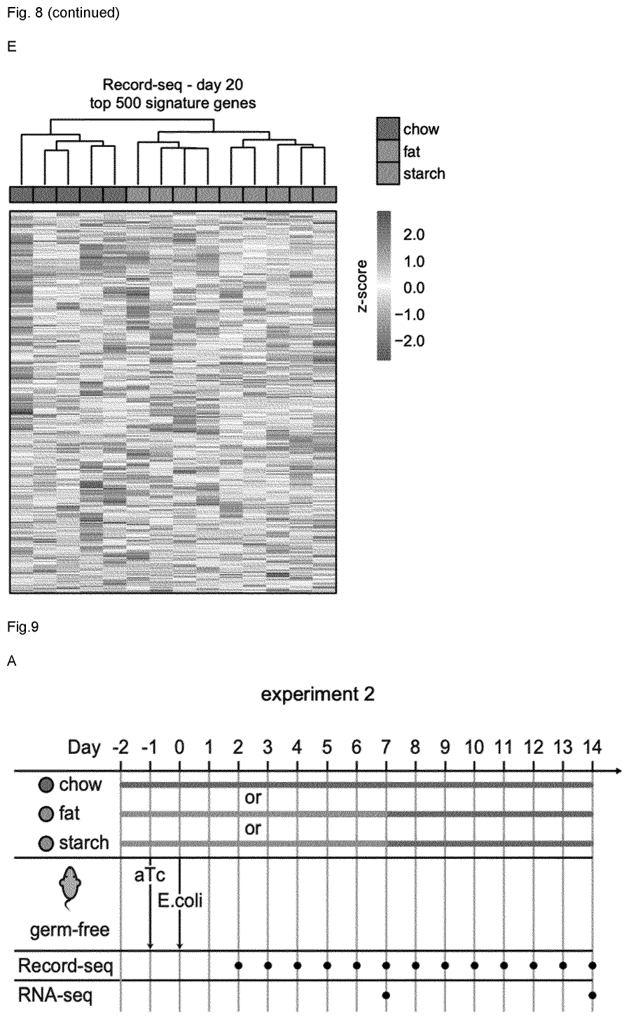

The inventors next assessed the capacity of transcriptional recording sentinel cells to record differences in the intestinal environment by varying the animals' diet. Mice monocolonized with sentinel cells were fed one of three diets: a standard chow or a purified diet based on starch or fat (referred to as starch or fat diets below). Starting from day 7, all groups received the chow diet (FIG. 1D). RNA-seq and Record-seq of fecal samples enabled the characterization of transcriptional changes and the stability of the recorded information. Before the diet switch both RNA-seq and Record-seq readily distinguished the diet groups (FIG. 8, A and B). After the switch to chow, the transcriptional signatures corresponding to starch or fat diets were rapidly lost with RNA-seq but not Record-seq (FIG. 1, E and F, and FIG. 8C to E). Thus, whereas RNA-seq represents a snapshot measurement, Record-seq durably reveals past transcriptional information.

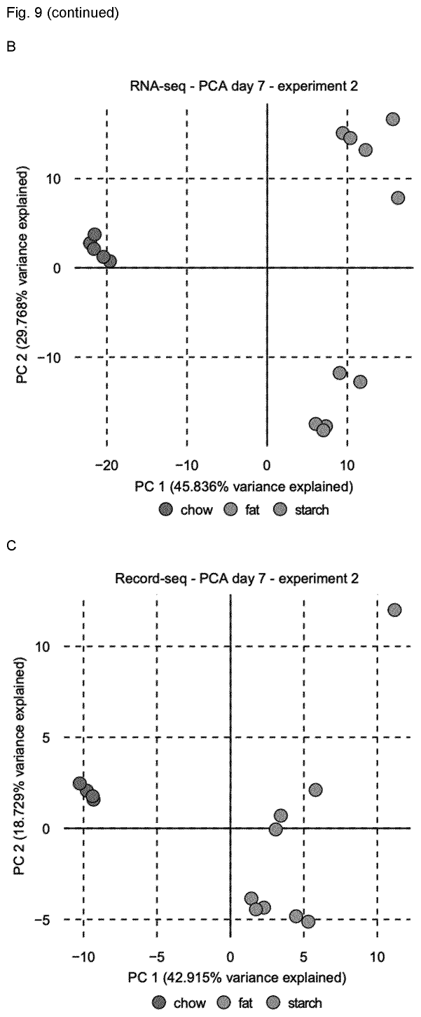

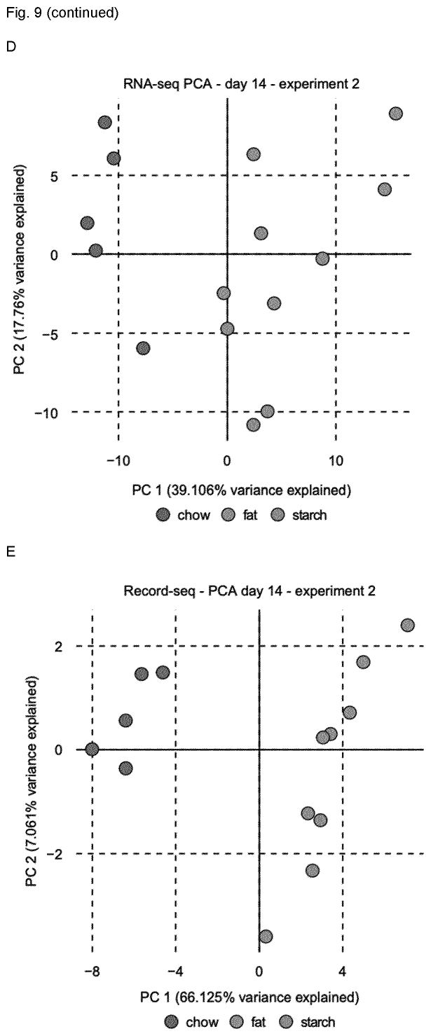

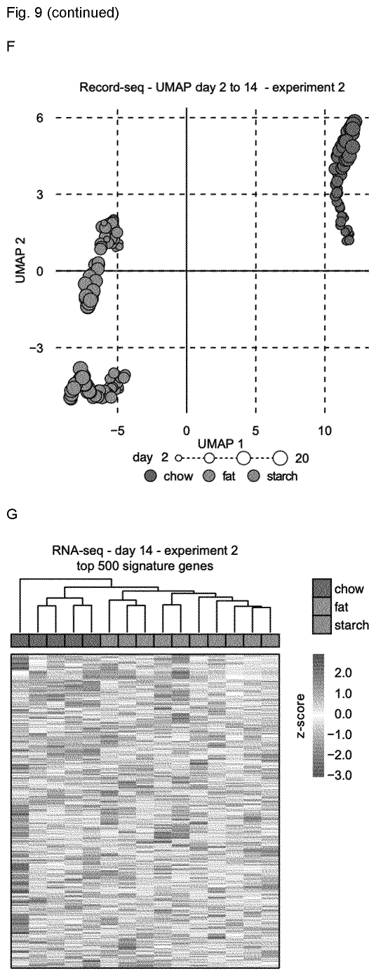

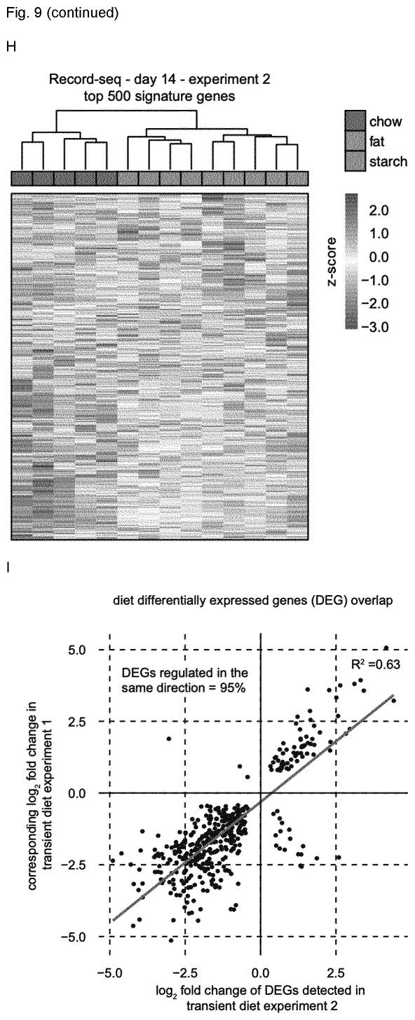

To verify the reproducibility of Record-seq, the inventors replicated the first 14 days in an independent experiment largely confirming the inventor's previous observations (FIG. 9A to H). The inventors also directly compared the two experiments and found a 95% overlap in the regulation of Record-seq differentially expressed genes (DEGs) (FIG. 91). Thus, transcriptional recording sentinel cells reproducibly record cellular transcriptomes and provide an archive of complex and dynamic features of different mammalian intestinal environments in vivo.

### Example 3: Record-Seq Reveals E. Coli's Adaptation to Intraluminal Conditions

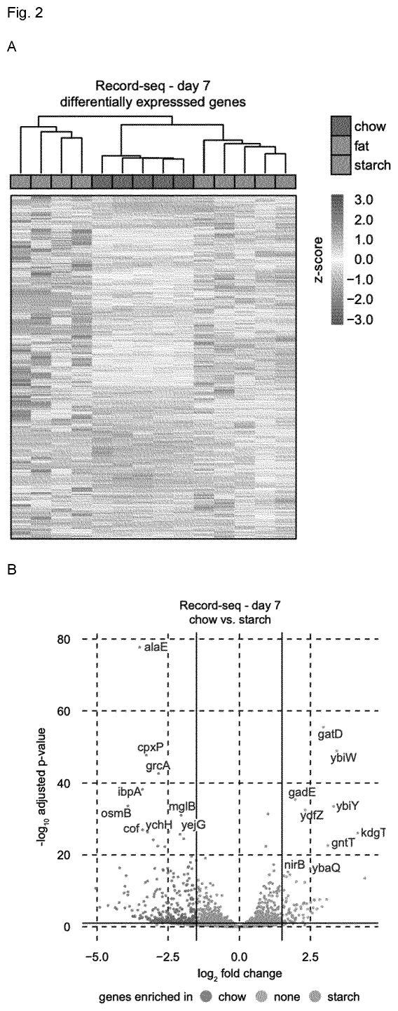

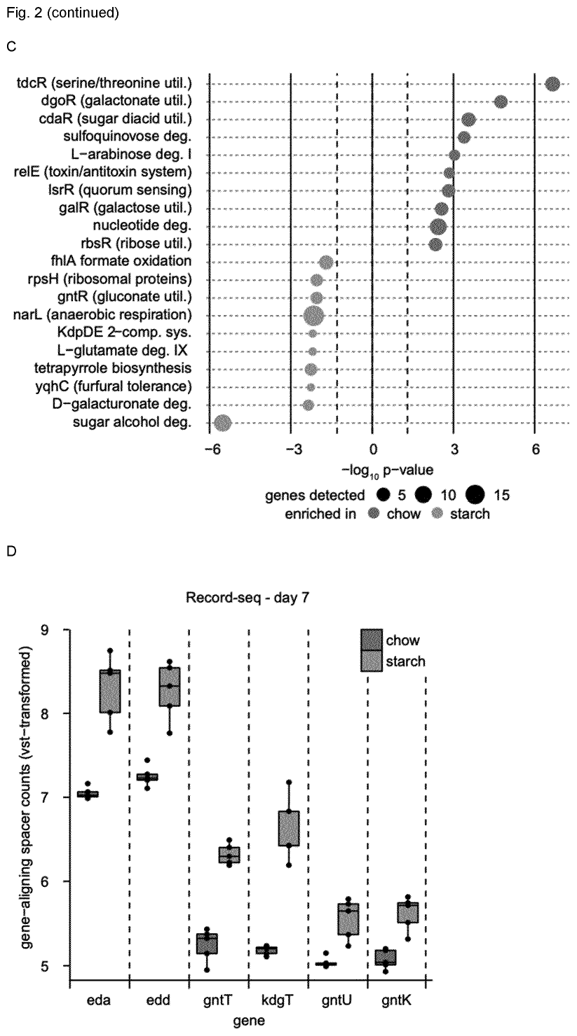

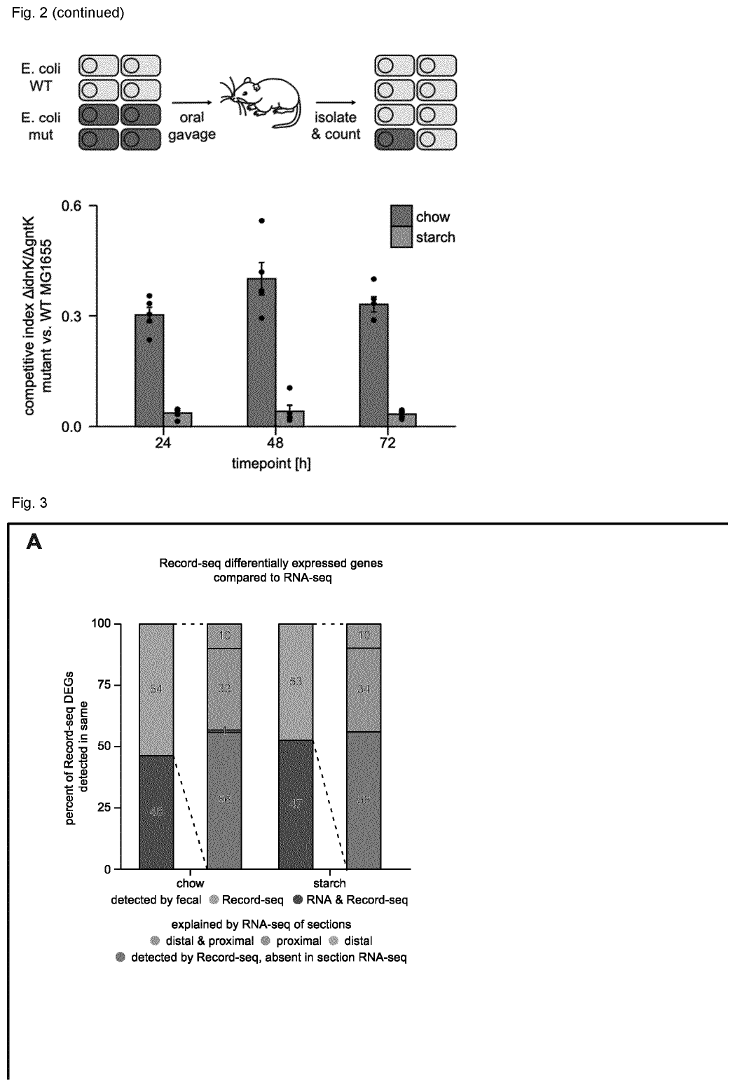

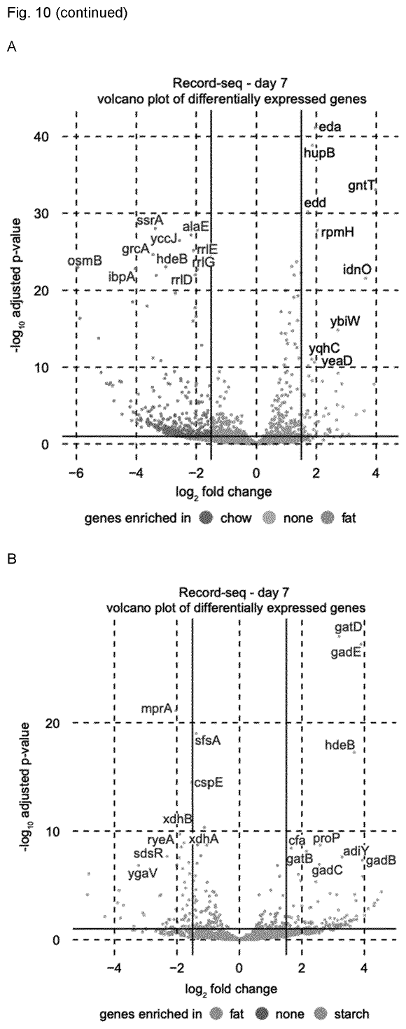

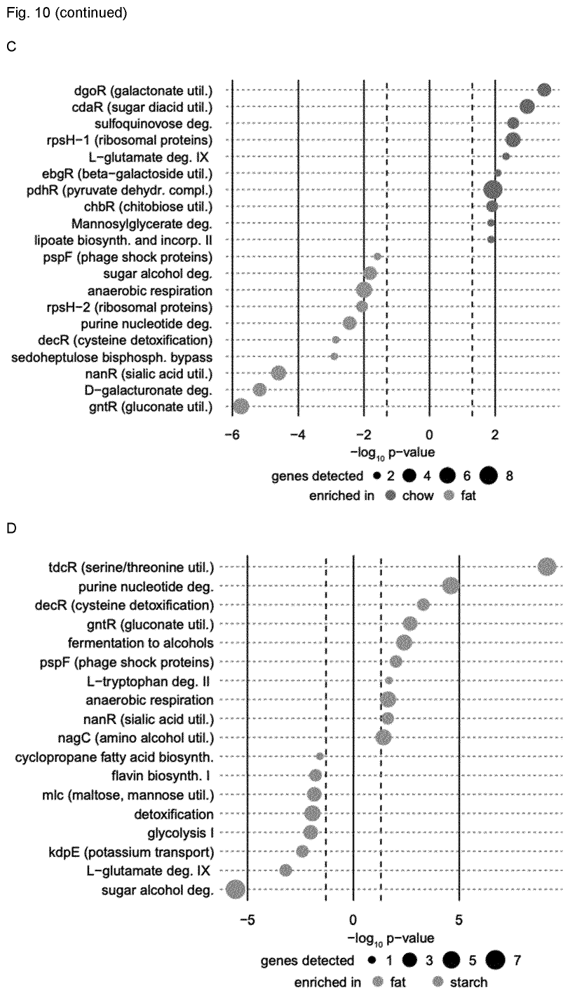

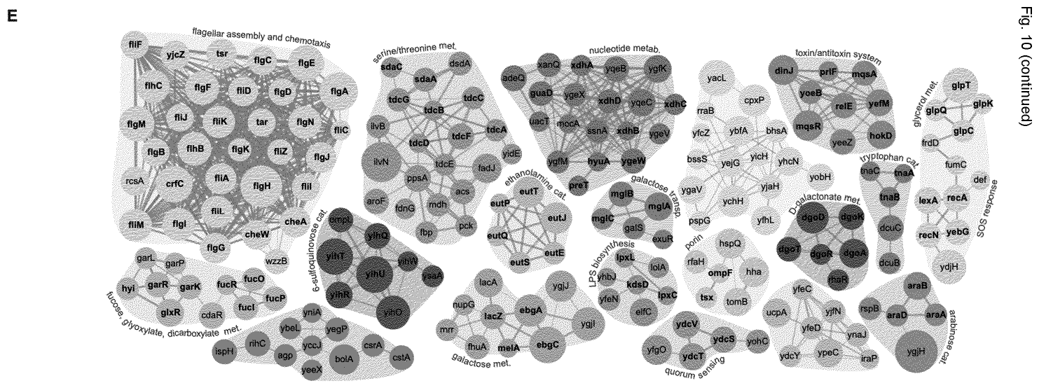

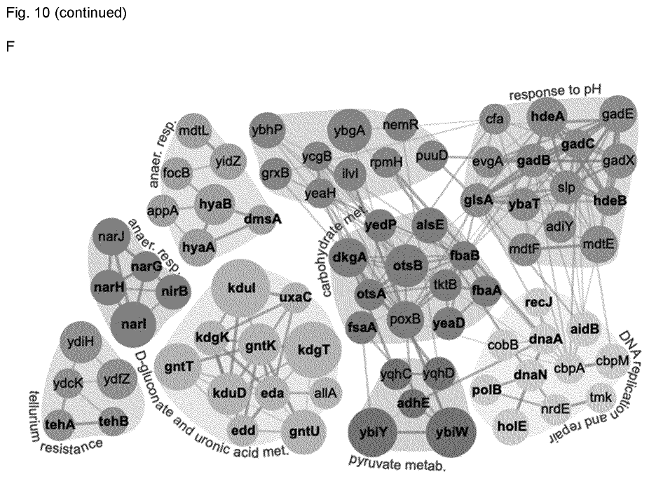

Record-seq characterization of the genes and pathways altered in E. coli's response to different diets delivered a detailed picture of E. coli's adaptation to intraluminal environments. DEGs readily classified the three diet conditions upon hierarchical clustering (FIG. 2A) and pairwise comparisons between each diet yielded hundreds of DEGs (FIG. 2B and FIG. 10, A and B).

Pathway enrichment analysis revealed a number of diet-dependent shifts in a wide range of cellular behaviors among the diet conditions (FIG. 2C and FIG. 10C and D). For further analysis, the inventors focused on the chow versus starch comparison. In starch-fed animals the inventors found signals indicative of nitrate use as an electron acceptor for anaerobic metabolism (narL), adaptation to low pH (gadC), potassium uptake (kdpDE), and carbohydrate utilisation. In chow-fed animals, E. coli used diverse carbon sources as suggested by overrepresentation of utilization pathways for galactonate, glucarate, galactarate, sulfoquinovose, arabinose, galactose, ribose, and fucose (FIG. 2C and FIG. 10E). These findings are in line with the diverse composition of the chow diet which is enriched in plant material, as well as the previously described adaptation of intestinal symbionts to diverse polysaccharide carbon sources.

Record-seq characterization of mice on the starch diet revealed the metabolic adaptation of E. coli in vivo to more restricted carbon source availability. The sugar acids galacturonate and gluconate are present in the host mucus and were shown to be used as carbon sources via the Entner-Doudoroff pathway (EDP) for E. coli colonization in streptomycin-treated mice. Here the inventors could use Record-seq directly in an unperturbed system where the only manipulation was a diet change, revealing that gluconate and galacturonate are alternative carbon sources in the face of nutritional limitation as shown by enrichment for their degradation pathways (FIG. 2C and FIG. 10F). These changes included key genes of gluconate metabolism which were strongly upregulated in E. coli from starch- versus chow-fed mice, such as the low and high affinity gluconate transporters (gntU and gntT, respectively), the permease for 2-keto-3-deoxygluconate (kdg7), gluconate kinase (gntK) as well as the central enzymes of the EDP (edd, eda) (FIG. 2D).

To confirm gluconate carbon source adaptation directly, the inventors carried out competitive colonizations with wild-type (wt) E. coli MG1655 and mutant E. coli MG1655 AgntK/AidnK (FIG. 2E). This mutant is unable to catabolize gluconate as it lacks both isoforms of the gluconate kinase, so the inventors expected it to suffer from a substantial competitive disadvantage compared to wt cells. Strikingly, the competitive disadvantage of E. coli MG1655 ΔgntK/ΔidnK over the wt was nearly 10-fold higher in starch- versus chow-fed mice (FIG. 2E)—suggesting that E. coli becomes more dependent on sugar acids from the host mucus purely due to carbon source changes. Thus, Record-seq delivers a detailed assessment of individual genes and entire pathways altered in E. coli's adaptation to different diet-dependent intraluminal environments without confounding manipulations.

### Example 4: Sentinel Cells Capture the Intestinal Milieu and Preserve Transient Features of the Cecum and Colon

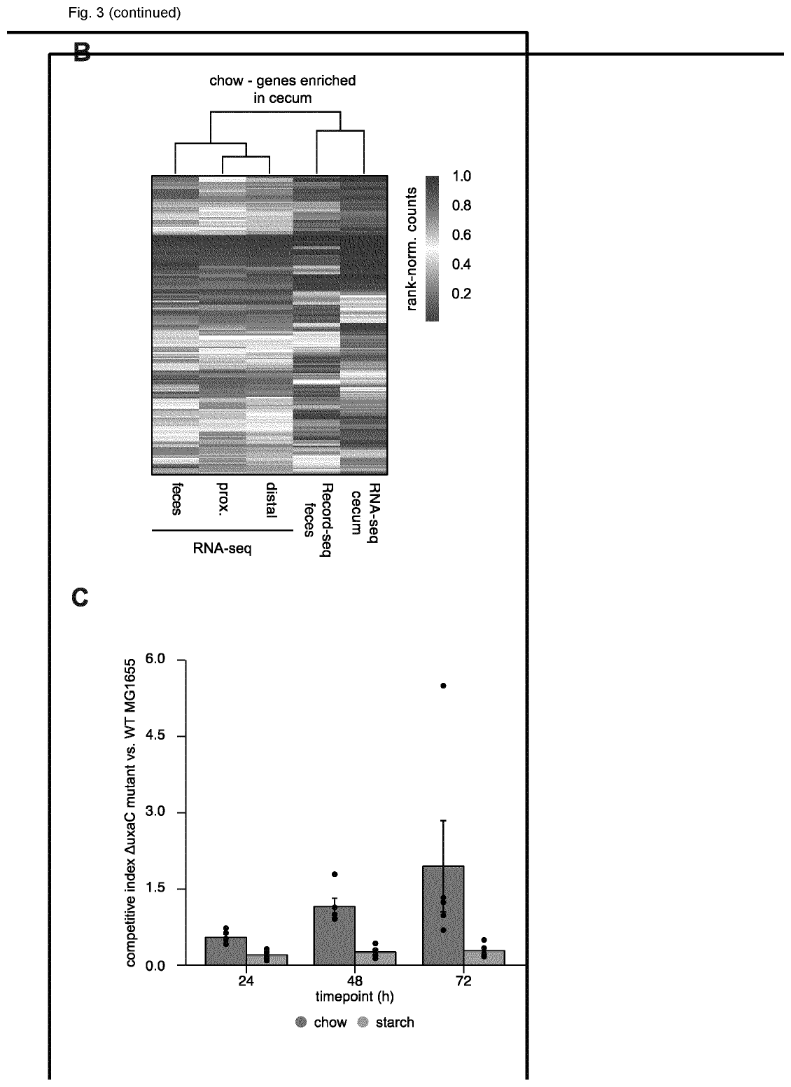

An important requirement of any intestinal reporting system that avoids potentially confounding manipulations or animal sacrifice is the ability to capture information from the microbial biomass of inaccessible proximal gut sections. Given the ability of Record-seq to preserve information over time, the inventors directly addressed whether the sentinel cells retain information along the longitudinal axis of the gastrointestinal tract by comparing fecal Record-seq with RNA-seq of E. coli from different intestinal segments of mice fed a chow or starch diet. The inventors found that ˜46% of the fecal Record-seq DEGs were identified as differentially expressed in the same direction by fecal RNA-seq (FIG. 3A), highlighting concordance between the two technologies. However, ˜54% of the genes detected by fecal Record-seq as upregulated under the chow or starch diet were found to either be oppositely regulated or show no differences between the diets in fecal RNA-seq (FIG. 3A).

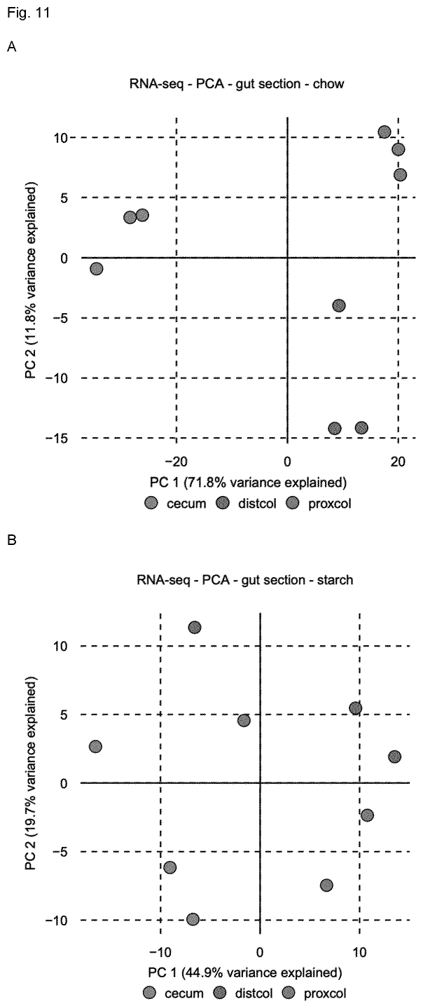

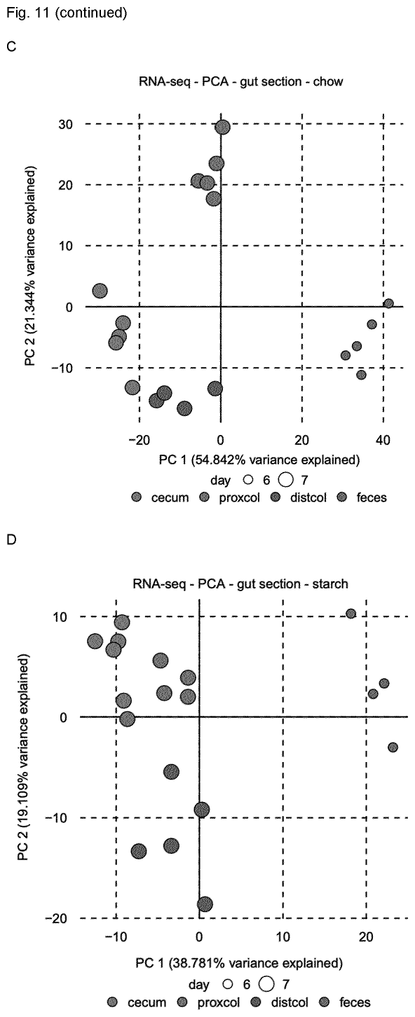

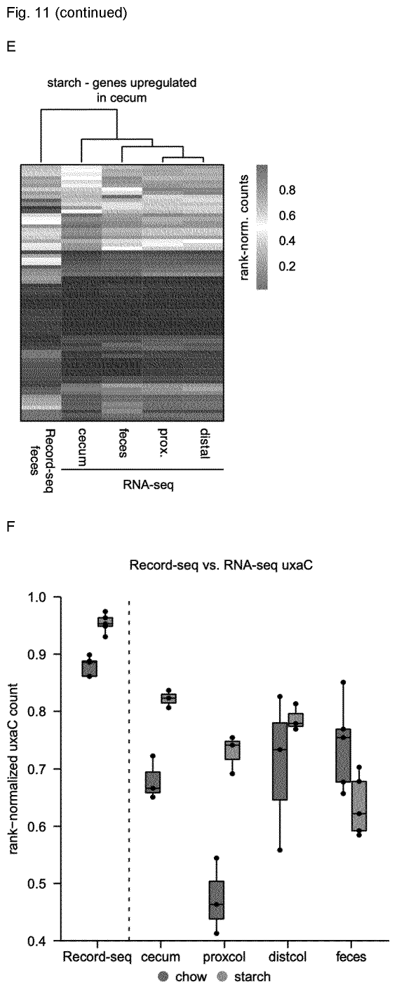

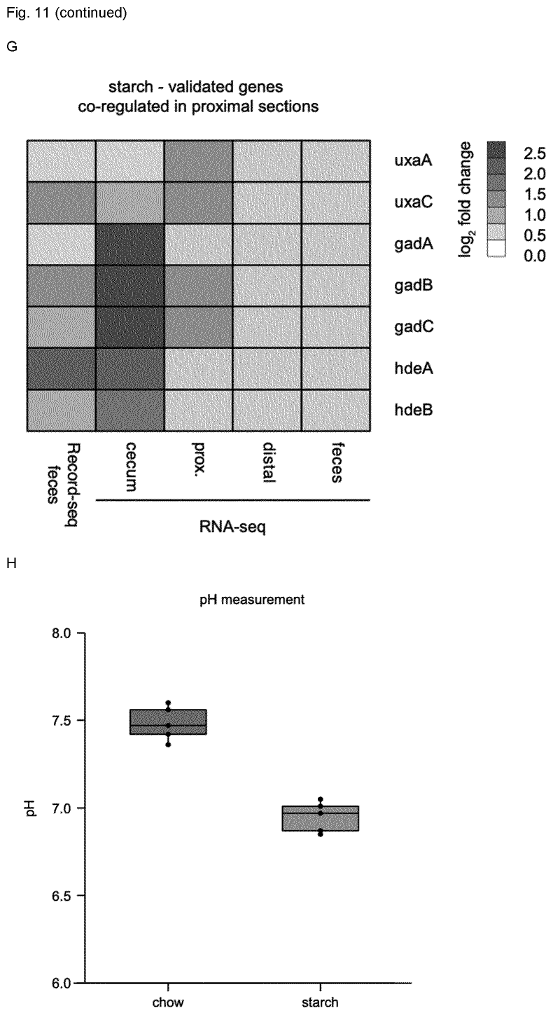

The inventors considered that fecal Record-seq captures transient events in proximal sections that are absent from fecal RNA-seq. After using RNA-seq to confirm that the transcriptional states of E. coli from different gut segments or feces were distinct (FIG. 11, A to D), the inventors addressed the origin of the 54% of genes unique to Record-seq in the chow condition (FIG. 3A). Indeed, a substantial proportion (33%) of these genes were explained by segment-specific RNA-seq signals in the cecum and proximal colon, and a further 10% were explained by expression in the cecum and throughout the colon. Only very few (1%) were unique to the distal colon. The remaining 56% identified by Record-seq but not RNA-seq were potentially differentially expressed in niches not covered by the sampling performed here (e.g. the small intestine or colonic mucus) or reflect successive transcriptional events that fall below significance thresholds in RNA-seq. Results from the starch diet were comparable to these findings although fewer genes were selectively expressed proximally, likely due to transit effects arising from differences between the fiber content of the diets (FIG. 3A). Rank-based hierarchical clustering performed on genes overexpressed in the cecum compared to other sections revealed better cecal RNA-seq clustering with Record-seq than with any other RNA-seq dataset, confirming that proximal information is preserved with Record-seq (FIG. 3B and FIG. 11E).

To validate a subset of insights regarding proximal gut sections revealed by fecal Record-seq, the inventors followed up on two findings using independent methodologies. First, uxaC—part of the hexuronate catabolism pathway—was uniquely upregulated in fecal Record-seq and proximal gut RNA-seq under the starch diet (FIG. 11, F and G), suggesting hexuronates are a preferred carbon source for E. coli under these conditions. In a competitive colonization between wt E. coli and a mutant deficient in hexuronate catabolism (ΔuxaC), the ΔuxaC strain indeed exhibited a significantly greater competitive disadvantage in starch-compared to chow-fed mice (FIG. 3C). Second, acid-stress-response genes (gadABC, hdeAB) were selectively overexpressed in fecal Record-seq and proximal gut RNA-seq under the starch diet (FIG. 11G). Accordingly, the pH of the cecum under the starch diet was significantly reduced compared to the chow diet (FIG. 11H). Thus, fecal Record-seq reports transcriptional events throughout the length of the intestinal tract and contains information absent from fecal RNA-seq analysis.

### Example 5: Record-Seq Provides a Non-Invasive Assessment of Intestinal Inflammation

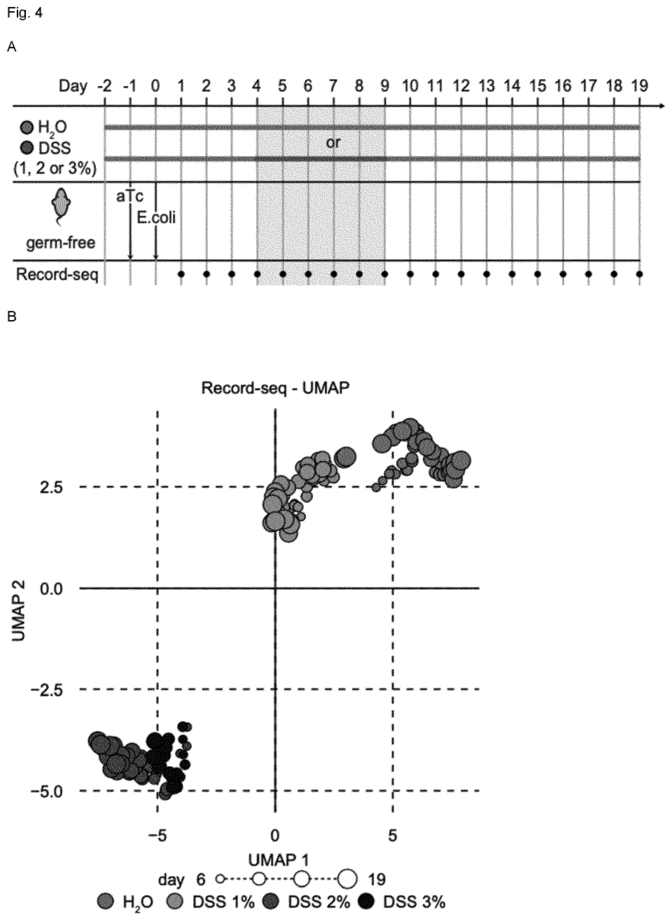

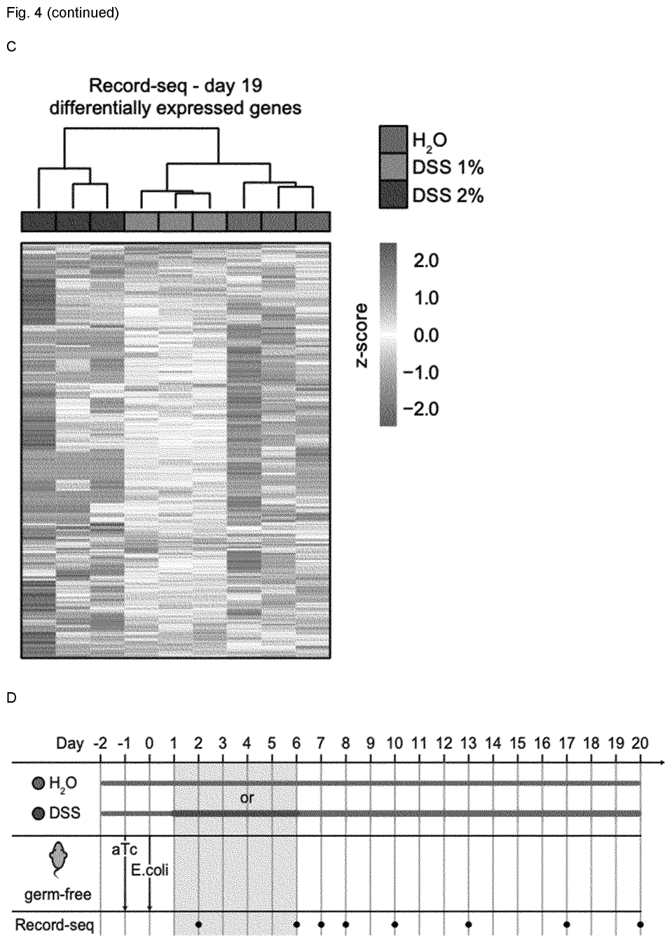

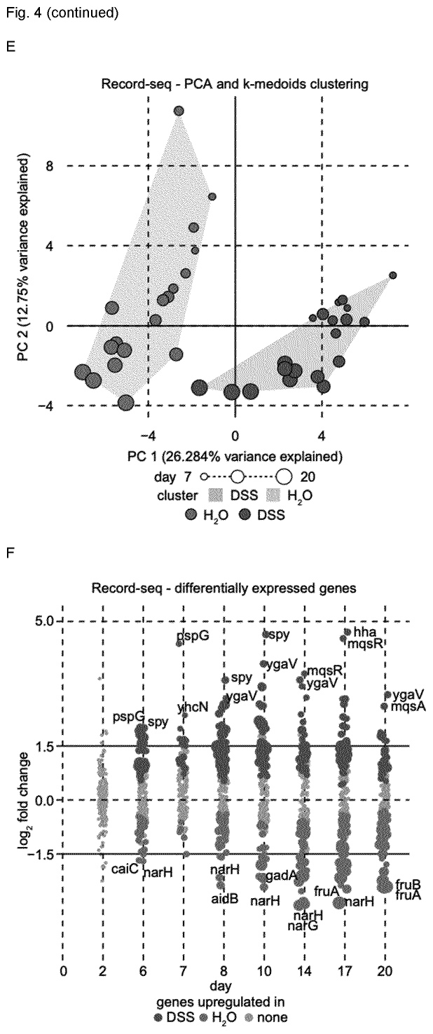

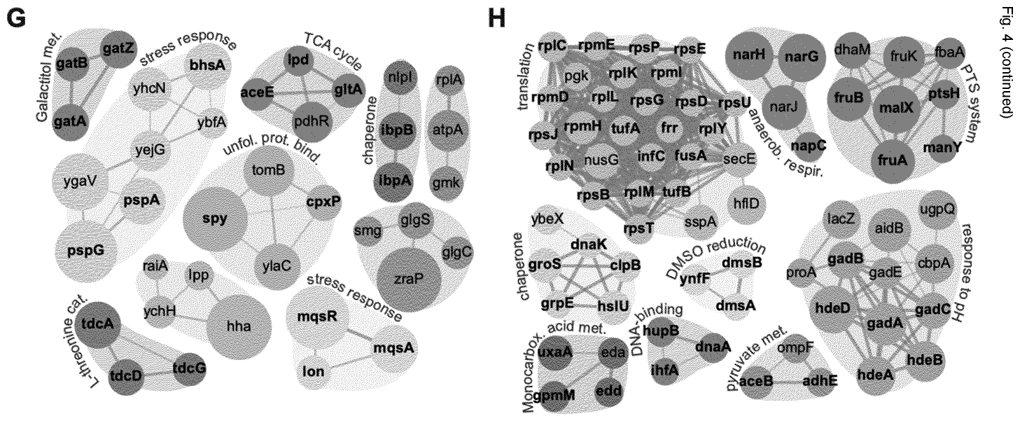

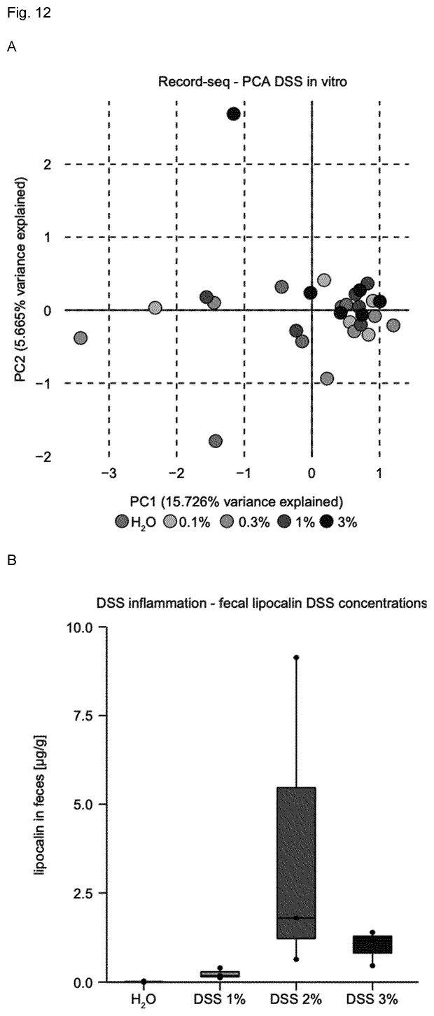

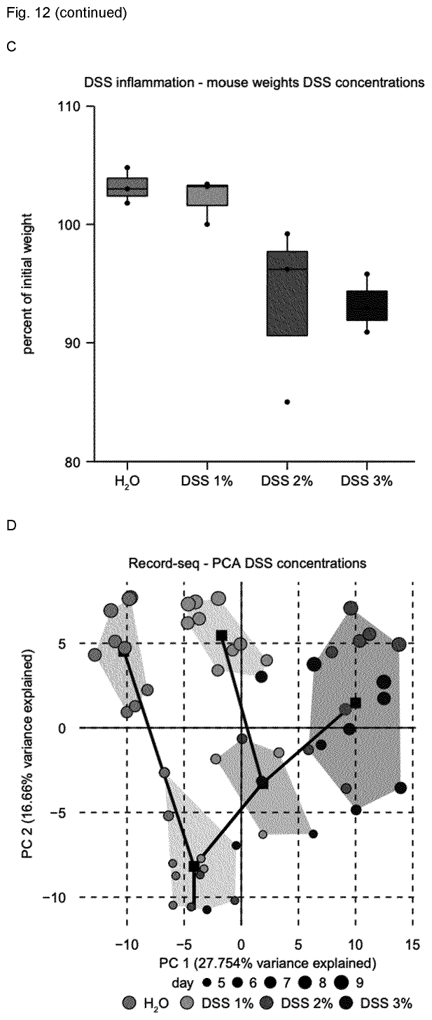

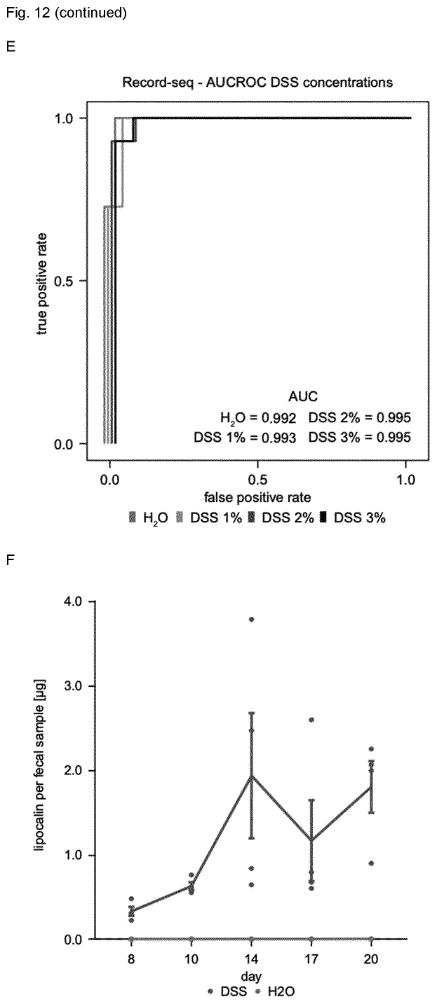

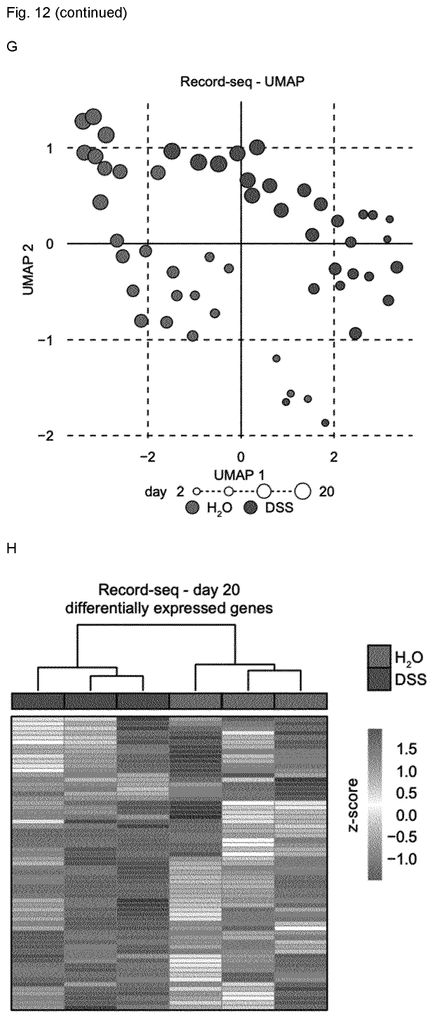

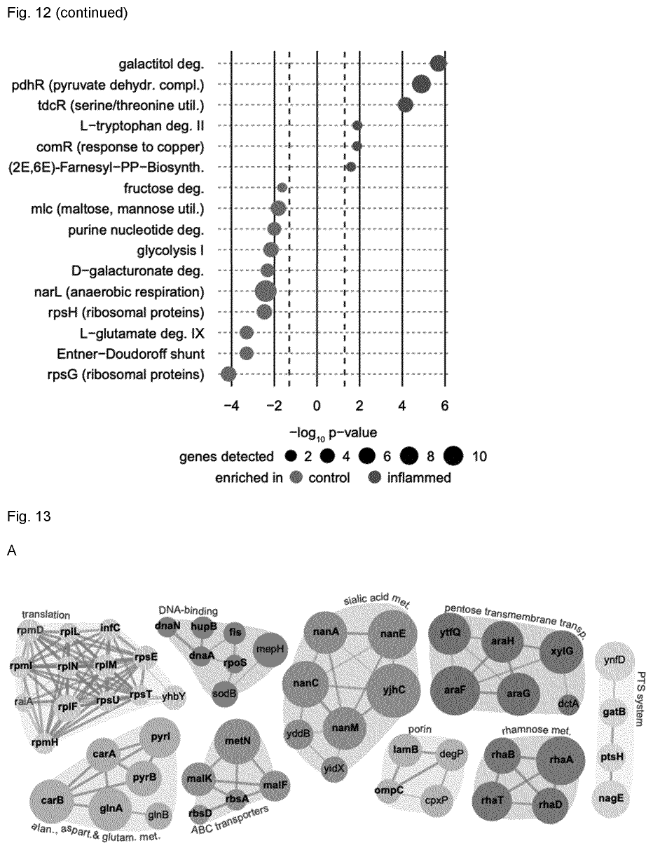

Another potential application of sentinel cells is their use as non-invasive living diagnostics capable of reporting on gastrointestinal diseases. To test this concept, the inventors used the dextran sodium sulfate (DSS)-induced colitis mouse model. After confirming that DSS had negligible direct impact on the E. coli transcriptome in vitro (FIG. 12A), the inventors performed Record-seq on sequentially collected fecal samples from mice treated with increasing concentrations of DSS (FIG. 4A). Record-seq not only distinguished treated versus control mice during DSS exposure and withdrawal but could also accurately report on the phenotypic severity of the colitis model (FIG. 4, B and C, and FIG. 12B to E).

Using a longer time course and only the 2% DSS condition, the inventors repeated the in vivo longitudinal recording experiment (FIG. 4D and FIG. 12F) to characterize the intraluminal changes resulting from DSS-induced inflammation throughout time. The inventors first confirmed that transcriptional records could distinguish DSS-treated and control mice and identify DEGs throughout the duration of the experiment (FIG. 4, E and F; FIG. 12, G and H). Next, the inventors analyzed the DEGs with EcoCyc pathway enrichment. Gene Ontology, KEGG, and STRING network tools during and following inflammation. This showed upregulation of genes required for bacterial membrane integrity (pspG) and post-stress persister cell formation (mqsAR, hha) (FIG. 4, F to H; FIG. 121). The inventors also observed upregulation of chaperones (spy and cpxP) and heat-shock proteins (ibpA and ibpB) that are induced by oxidative stress. By contrast, genes and pathways most prominently downregulated in DSS-induced colitis included those required for the nitrate and DMSO electron acceptor pathways (narGH, dmsAB), adaptation to low pH (gad), and the Entner-Doudoroff pathway (edd, eda). Downregulation of the latter under inflamed conditions likely senses the mucus depletion that characterizes intestinal inflammation. These findings also suggest that E. coli shows decreased anaerobic respiration during inflammation but increased levels of envelope and oxidative stress likely resulting from augmented luminal oxygenation. Several ribosomal protein operons were also downregulated under DSS inflammatory conditions. Transcription of ribosomal proteins is regulated by ppGpp and DksA as part of the stringent response to nutrient limitation. The inventors concluded that Record-seq sentinel cells can accurately report on DSS-induced colitis disease severity while simultaneously revealing multiple features of the intestinal inflammatory environment.

### Example 6: Record-Seq Illuminates Both Host-Microbe and Microbe-Microbe Interactions

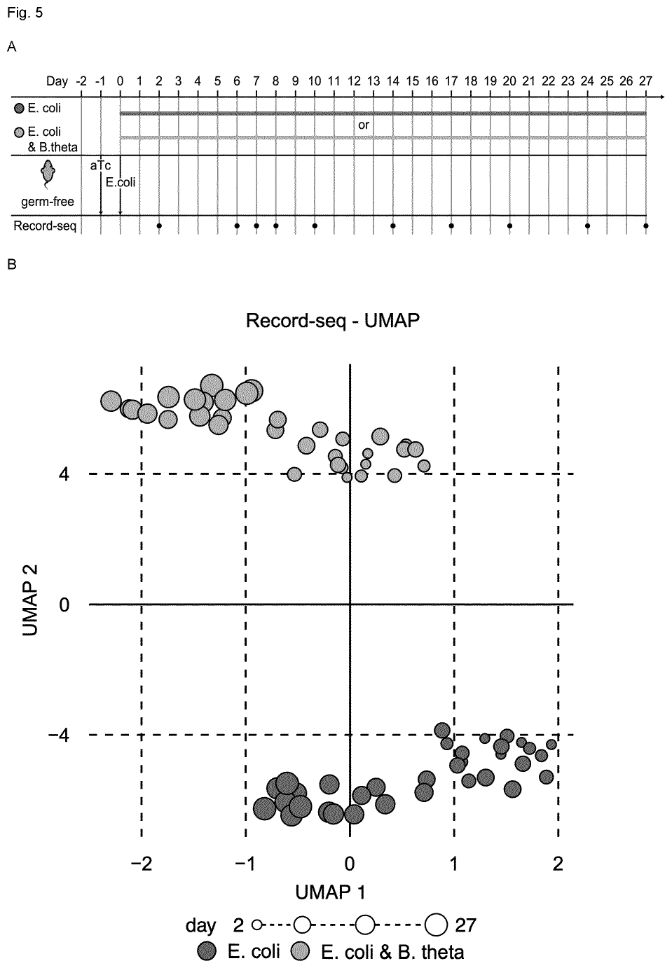

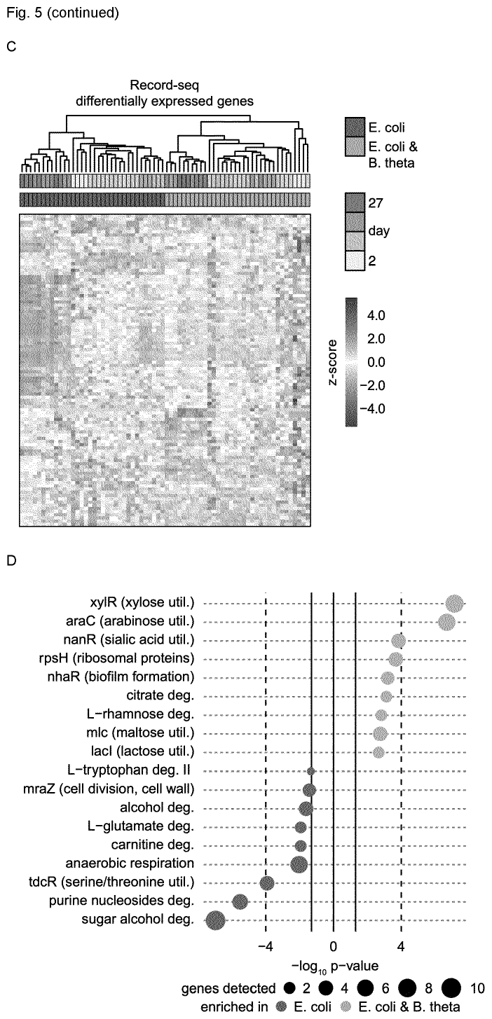

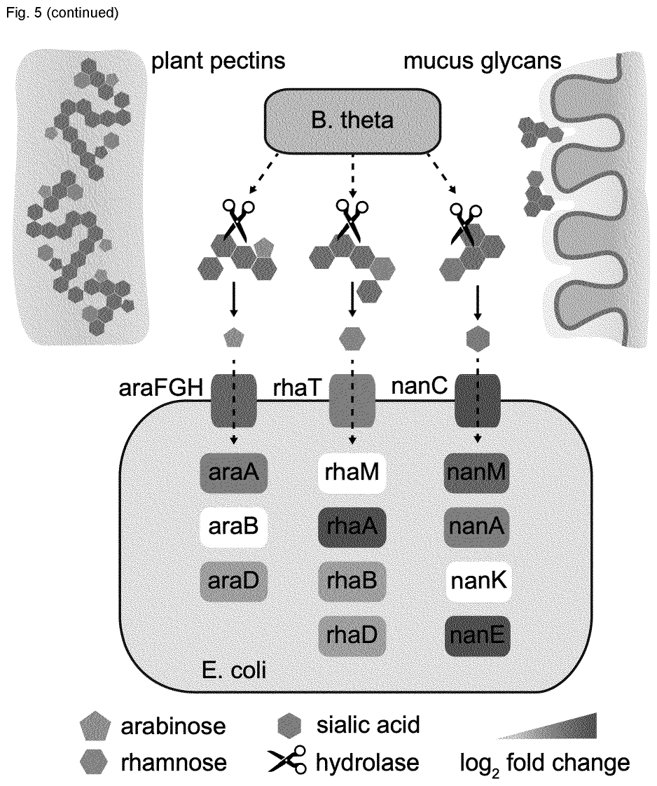

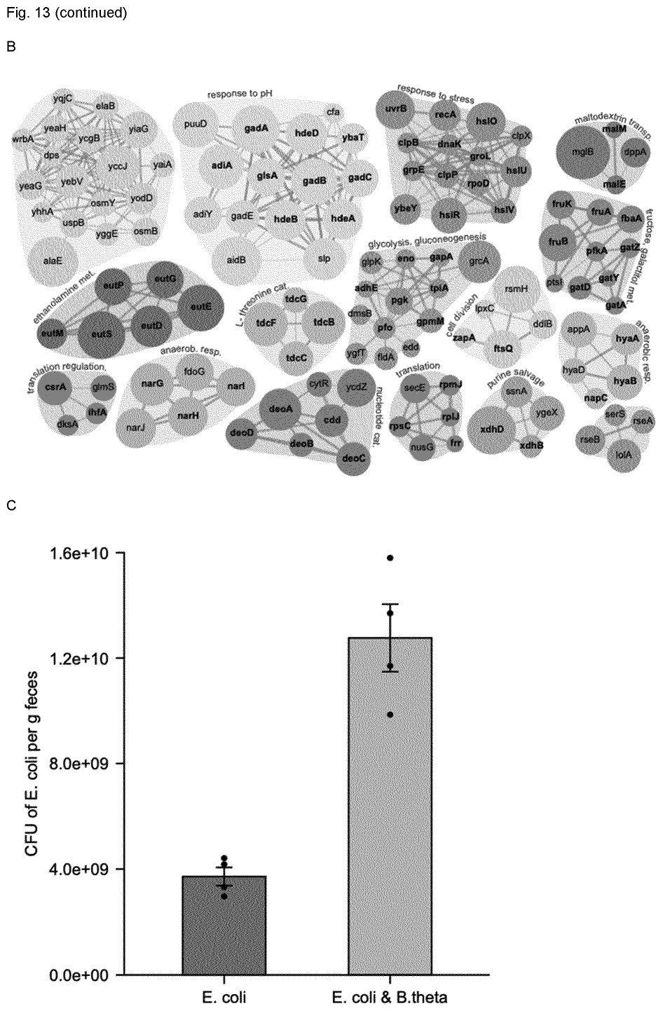

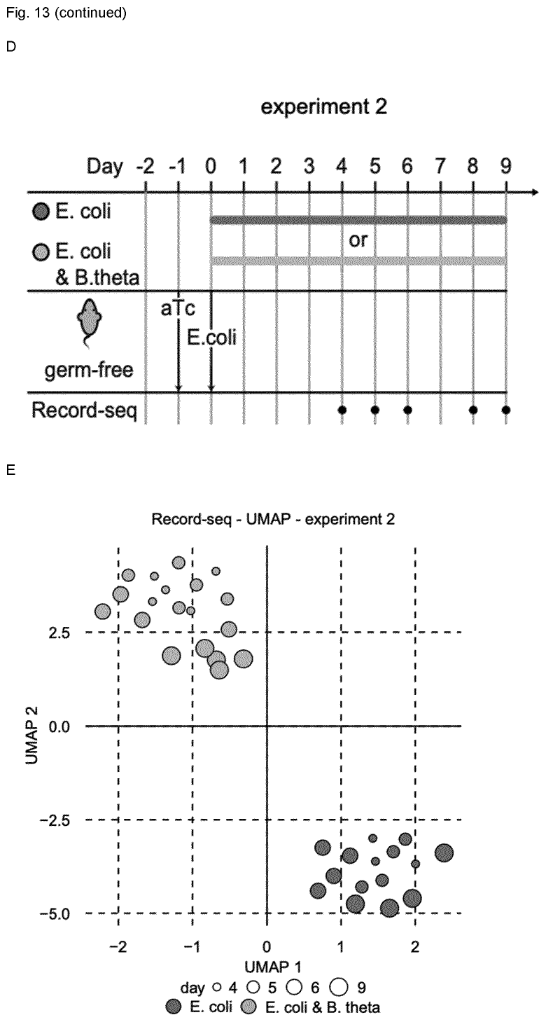

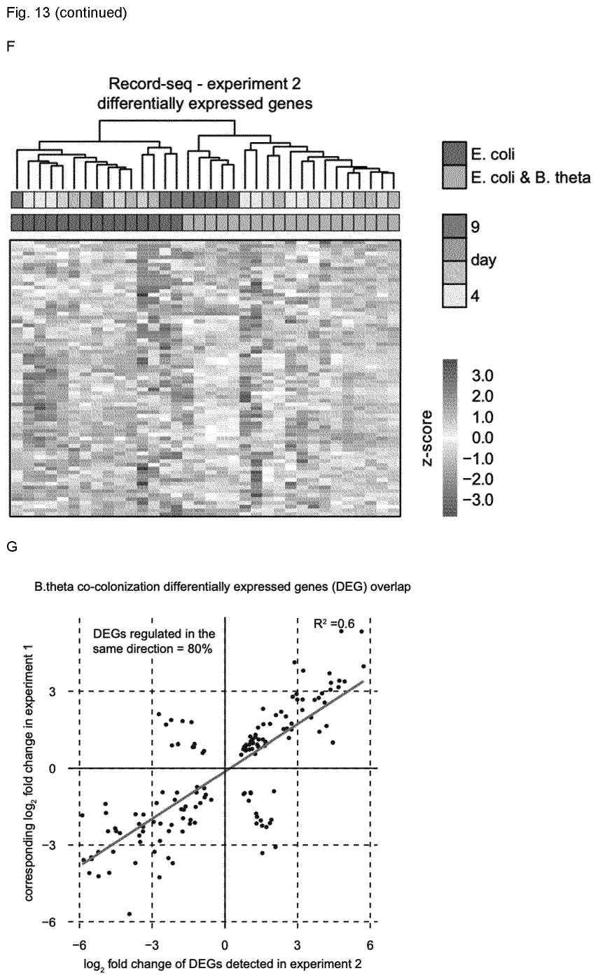

The capability to obtain transcriptional profiles from proximal parts of the microbial biomass non-invasively and longitudinally within the same host is potentially valuable for both animal research and in future applications in humans. To assess the performance of Record-seq in the presence of other intestinal microbes, the inventors started by performing longitudinal in vivo recording experiments in mice in the presence of one other prototypical member of the human gut microbiota, Bacteroides thetaiotaomicron. Distinct transcriptional archives were obtained from mice either monocolonized with E. coli, or co-colonized with E. coli and B. thetaiotaomicron (FIG. 5, A to C). The transcriptional records revealed alterations over a wide range of microbial functions, including a dramatic shift in inferred E. coli carbon source preferences (FIG. 5D; FIG. 13, A and B). In the presence of B. thetaiotaomicron, pathways for utilization of xylose, arabinose, sialic acid, amino alcohols, citrate, rhamnose, maltose, and lactose were significantly upregulated. Given that B. thetaiotaomicron has a far richer content of polysaccharide utilization loci compared with E. coli and that mice were fed with chow containing complex plant cell wall carbohydrates, the inventor's data supports cross-feeding (FIG. 5E) by B. thetaiotaomicron liberating usable input nutrients (e.g. mono and oligosaccharides) from otherwise unmetabolizable complex diet- or host-derived materials (e.g. plant pectins and mucus glycans). Supporting the notion that nutrient cross-feeding is beneficial for E. coli, the inventors observed a 3.4-fold increase in E. coli biomass in the presence of B. thetaiotaomicron compared to monocolonized mice (FIG. 13C).

The presence of B. thetaiotaomicron also led to downregulation of E. coli genes involved in metabolism of sugar alcohols, amino acids, fructose, nucleotides, and ethanolamine, suggesting that these secondary carbon sources were not required during bicolonization (FIG. 5D and FIG. 13B). Selective expression of ribosomal proteins when E. coli co-colonized with B. thetaiotaomicron (FIG. 5D and FIG. 13A) is likely indicative of a stringent response on non-preferred carbon sources when E. coli colonizes alone. These results, which the inventors validated independently (FIG. 13, D to G), demonstrate that Record-seq sentinel cells sense alterations in the intestinal environment in the presence of another taxon and report on the transcriptional adaptations that occur as a result.

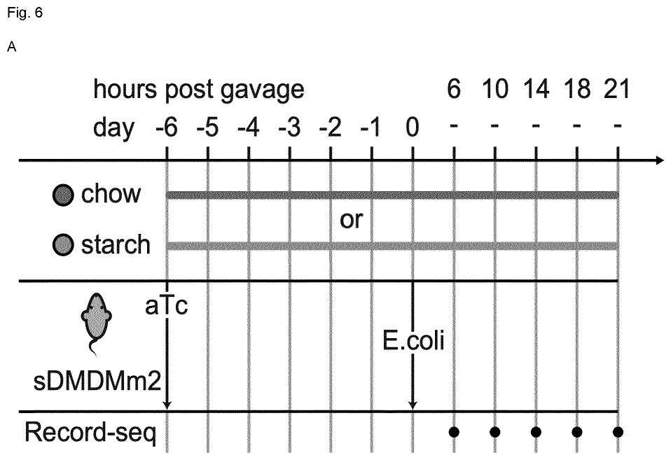

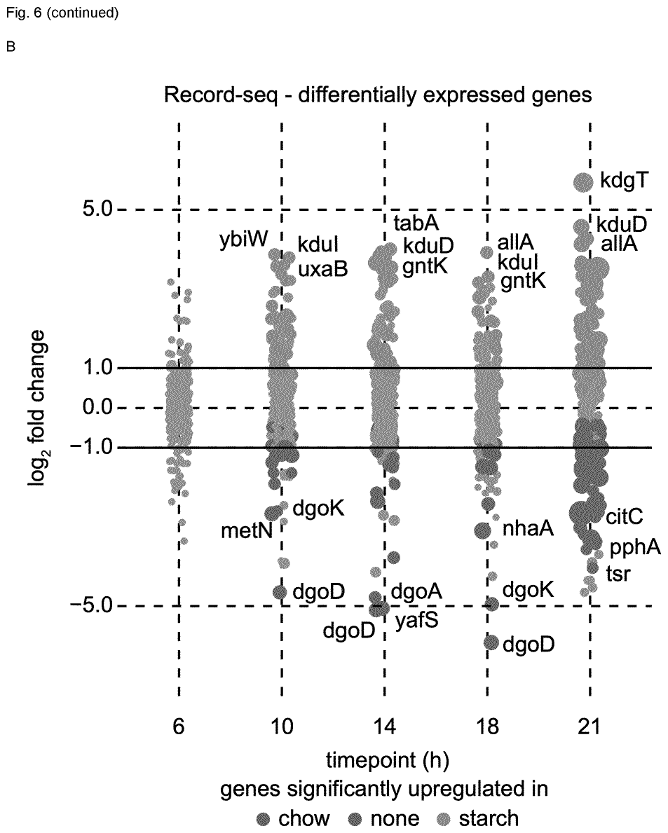

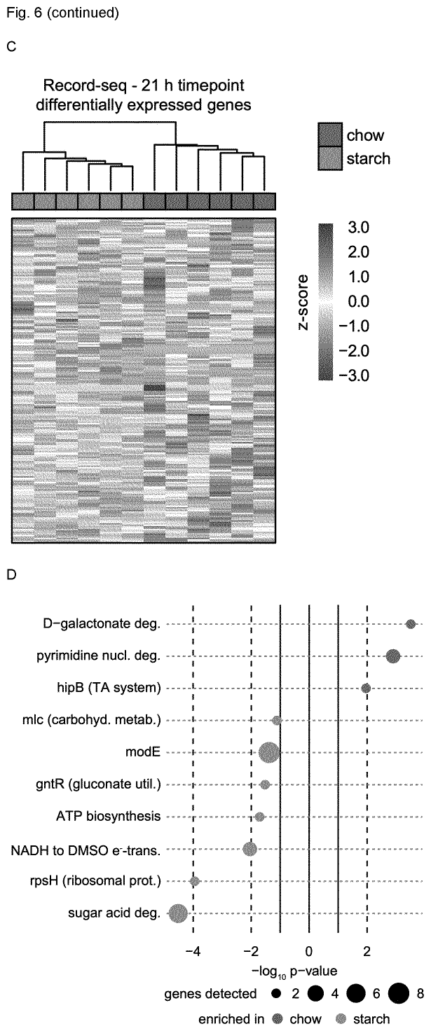

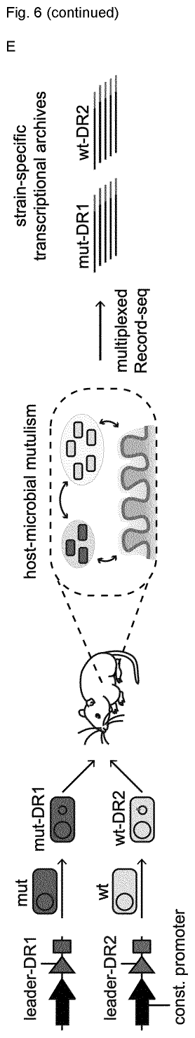

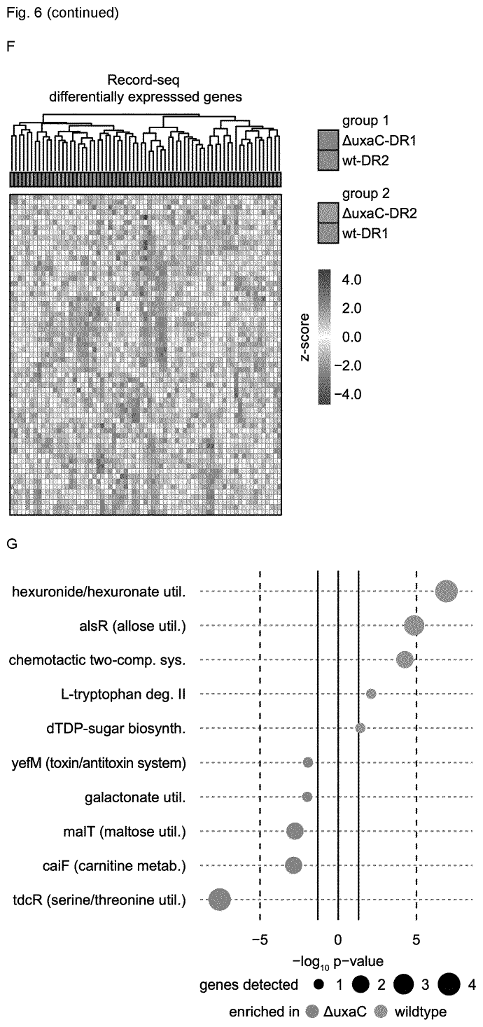

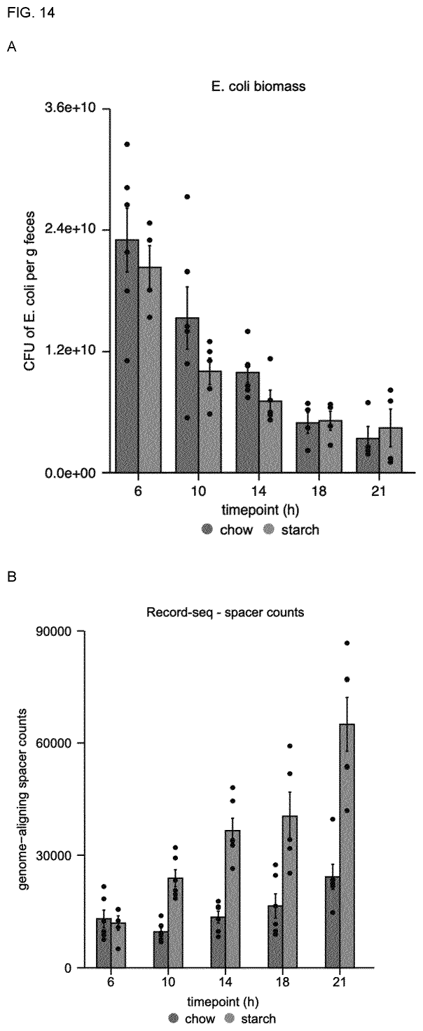

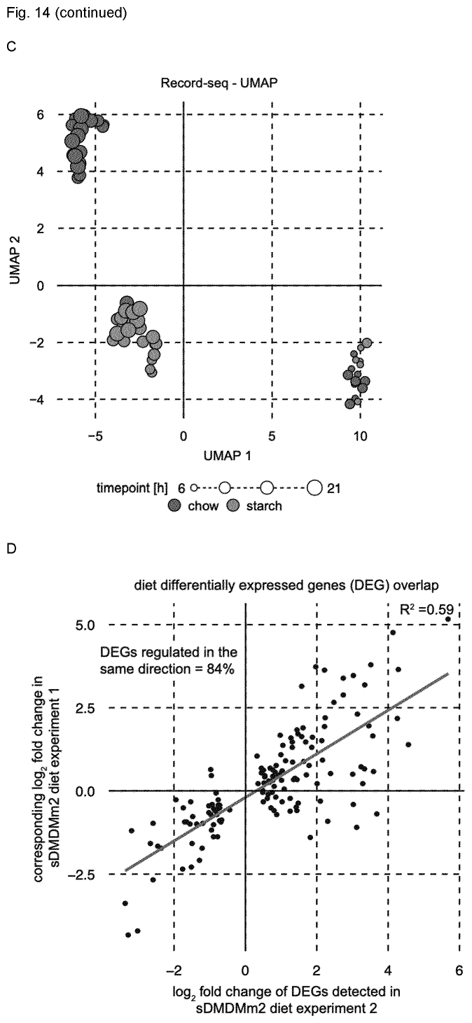

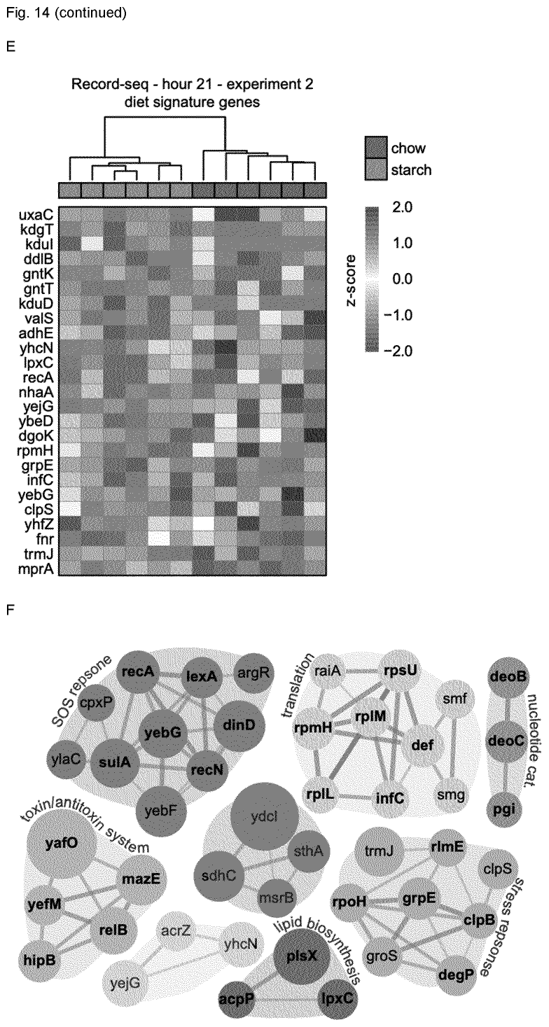

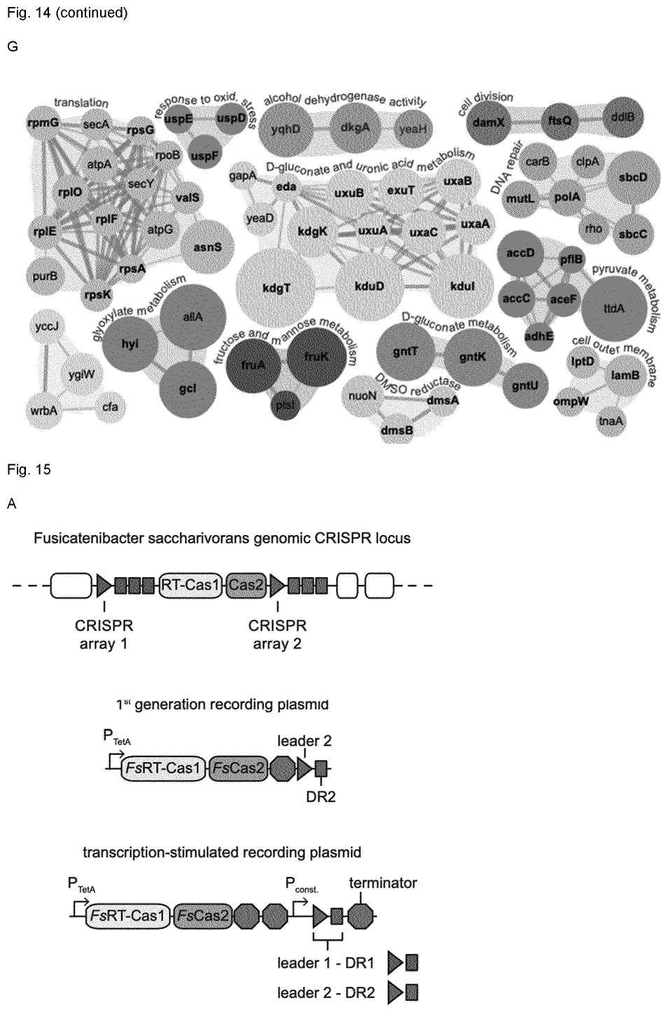

The inventors next assessed the capacity of Record-seq to characterize gut function in the presence of a complex microbiota by gavaging sentinel cells into chow or starch fed mice colonized by a defined 12-member sDMDMm2 consortium (FIG. 6A). Although colonization resistance caused most gavaged E. coli sentinel cells to rapidly pass through the gastrointestinal tract (FIG. 14A), the inventors detected transcriptional records as early as 6 hours after gavage (FIG. 14B) and differential expression according to diet from 10 hours post gavage (FIG. 6B). Additionally, the recorded information was sufficient for stratifying the diet groups (FIG. 14C) and reproduced in an independent experiment (FIG. 14D). By 21 hours, Record-seq detected 220 DEGs in E. coli passing through sDMDMm2-colonised mice on a chow versus starch diet (FIG. 6C). STRING network, KEGG/GO and EcoCyc pathway enrichment analysis revealed diverse features of E. coli's adaptation to these ecologically complex environments including diet-dependent alterations in carbon metabolism, anaerobic versus aerobic energy harvesting and stress responses (FIG. 6D; FIG. 14E to G). An example was galactonate metabolism. Since none of the bacterial species from the sDMDMm2 microbiota encode a complete DeLey-Doudoroff galactonate degradation pathway, the finding of prominent upregulation of genes involved in galactonate metabolism in E. coli from chow-fed sDMDMm2 mice indicates that galactonate is likely available as a substrate for E. coli in this in vivo microbial consortium. Consistent with the inventor's earlier monocolonisation results (FIG. 2C), E. coli from starch-fed sDMDMm2 mice overexpressed genes for utilization of mucus-derived sugar acids such as hexuronates and gluconate (FIG. 14, F and G), supporting the interpretation that host-derived sugar acids become more important even in the presence of an unmanipulated gnotobiotic microbiota as the dietary nutrient sources become less rich. Thus, transcriptional recording sentinel cells function in the context of an intestinal microbiota to reveal a portfolio of host- and microbe-microbe interactions.

### Example 7: Multiplexed Record-Seq Enables Parallel Transcriptional Profiling of Isogenic Bacterial Strains Coinhabiting the Mouse Intestine

Although genetic polymorphism between taxa potentially allows RNA-seq to distinguish the transcriptional profiles of different taxa within an intestinal consortium, it is not informative of adaptive transcriptional differences between isogenic strains of the same taxon differing according to one genetic locus. The inventors hypothesized that Record-seq could meet this need, allowing a mechanistic understanding of how a particular genetic lesion is functionally compensated within a taxon when two strains are coinhabiting the intestine.

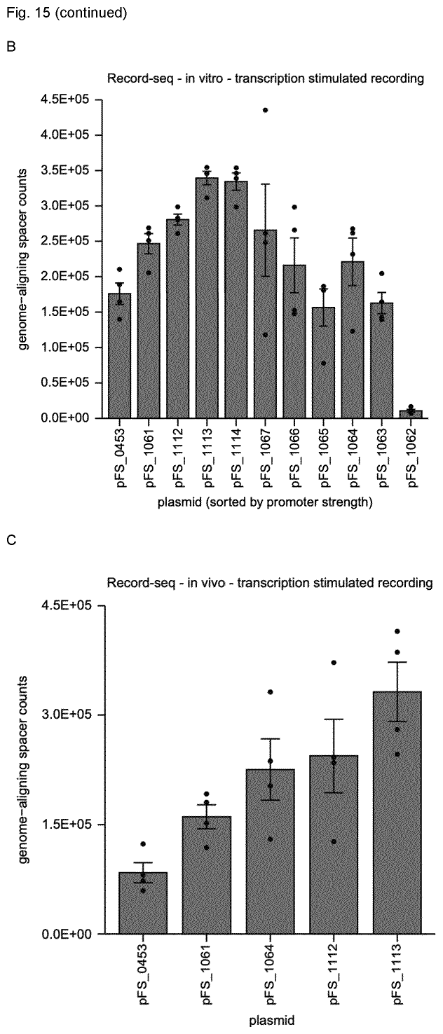

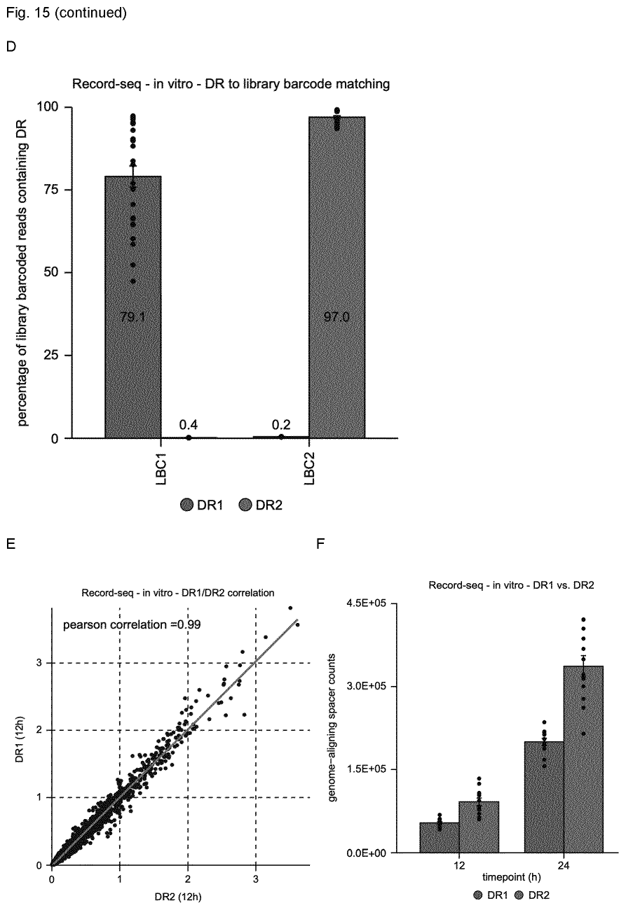

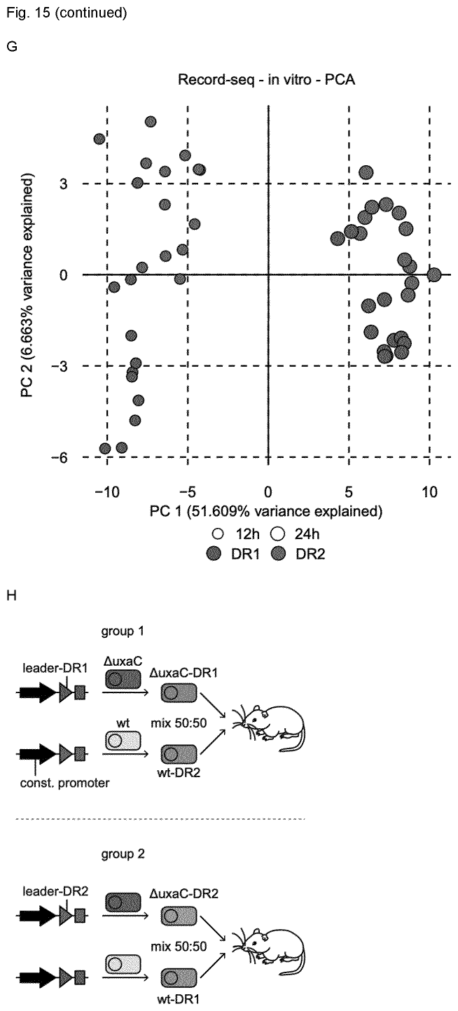

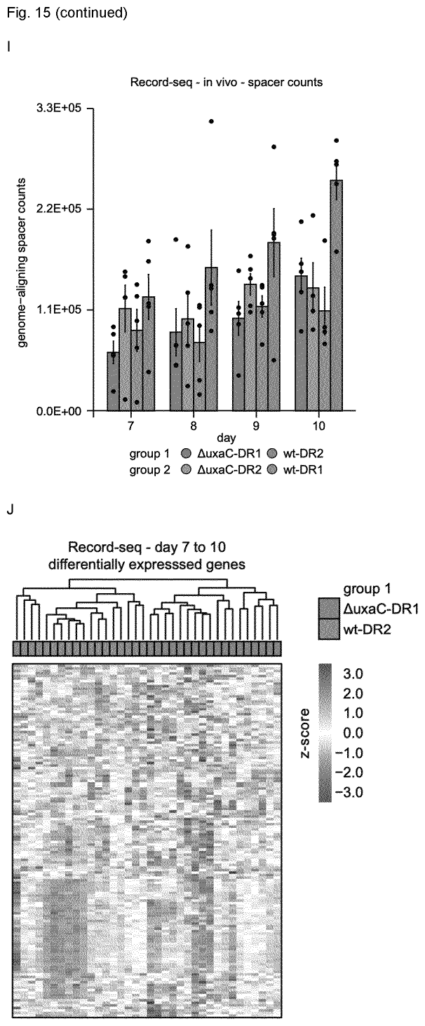

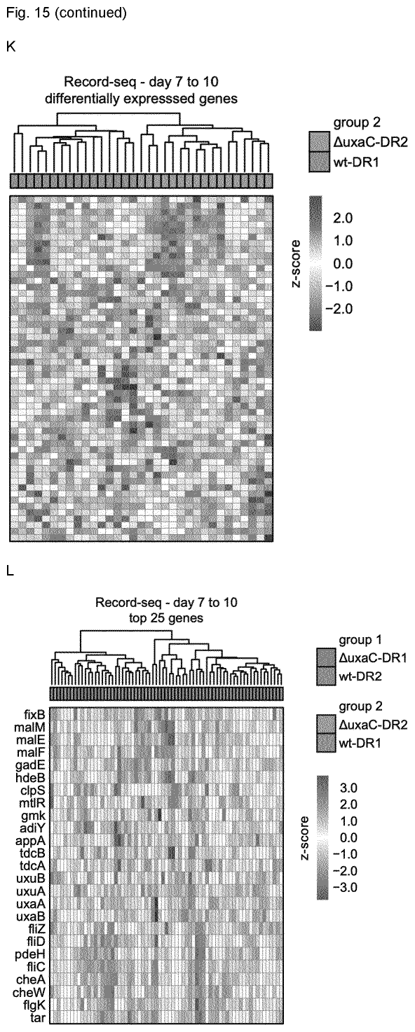

To test this concept, the inventors modified the Record-seq technology. First, the inventors leveraged recent insights revealing that CRISPR spacer acquisition is aided by transcription-coupled repair and introduced a constitutive promoter upstream of the CRISPR array within the recording plasmid, which improved recording efficiency. Second, the inventors developed multiplexed transcriptional recording (FIG. 7A) by using two orthogonal CRISPR arrays with distinct leader and direct repeat (DR) sequences, referred to here as DR1 and DR2. After confirming that the barcoded second-generation recording constructs facilitated labeling and computational stratification of the transcriptional archives of isogenic E. coli strains in vitro and in vivo (FIG. 15), the inventors investigated whether multiplexed Record-seq could reveal the compensatory mechanism of an isogenic single-gene mutant in a competitive setting in vivo. The inventors prioritized uxaC-deficient E. coli because Record-seq had revealed the importance of uxaC under the starch diet (FIG. 3C, FIG. 11F). In two independent experiments germ-free mice co-colonized with wt and uxaC-deficient (ΔuxaC) E. coli MG1655 harbored barcoded recording plasmids where wt-DR2 was in competition with ΔuxaC-DR1 (group 1) or conversely wt-DR1 competed with ΔuxaC-DR2 (group 2). Each isogenic strain revealed robust transcriptional recording activity in vivo and differences in the transcriptional signatures were driven by genotype (FIG. 7B), demonstrating multiplexed Record-seq transcriptional recording of two isogenic E. coli strains inside the same mouse intestine.

Analysis of DEGs and pathways revealed decreased expression by the uxaC-deficient mutant of other hexuronate utilization genes such as uxaA, uxaB, uxuA and uxuB in addition to the expected lack of uxaC (FIG. 7C). Since uxaA, uxaB and uxaC are induced by galacturonate and uxuA and uxuB by glucuronate, the wt strain likely displaces the ΔuxaC strain from niches where these sugar acids are available. Furthermore, the ΔuxaC strain appears to compensate for this deficiency by the upregulation of serine/threonine- and maltose-utilizing gene products. Thus, Record-seq has the unique capacity to enable multiplexed transcriptional profiling of two isogenic strains of E. coli in the same mouse gut. This approach reveals compensatory mechanisms in response to intra-species competition where RNA-seq based experiments are uninformative.

### Example 8: Transcriptional Recording Sentinel Cells Acquire Transcriptional Records within the Mouse Gut

The inventors extensively characterized the general properties of newly acquired spacers to assess whether any differences compared to the inventor's initial Record-seq experiments in vitro emerged. The inventors aligned newly acquired spacers to both the recording plasmid as well as the E. coli genome and found that the hallmarks of spacer acquisition the inventors had observed previously remained consistent. These are: acquisition of spacers from both plasmid and genome (FIG. 7D), preferential acquisition of spacers in an ‘antisense’ orientation (FIG. 7E) and a median spacer length of 41 bp (FIG. 7F). Furthermore, the median GC content of spacers was 40% (FIG. 7G) and the inventors did not find any preference for a specific sequence motif within or flanking the newly adapted spacer. Taken together, these data show that transcriptional recording by FsRT-Cas1-Cas2 is fully functional in the gut and important characteristics of acquired spacers were largely consistent with the inventor's in vitro study.

### Example 9: Record-Seq Enables Parallel Transcriptional Profiling of Isogenic Bacterial Strains Coinhabiting the Mouse Intestine

RNA-seq of complex intestinal consortia is performed by isolating and sequencing RNA from samples containing a mixed pool of bacteria (meta-transcriptomics). Differential gene expression analysis of individual members of the microbiota requires alignment of sequencing reads to the respective reference genomes. Close sequence similarity between multiple reference genomes can complicate this procedure, yielding a significant share of transcripts that align not uniquely to one but multiple reference genomes. Consequently. this approach does not allow to derive the individual transcriptional profiles of two isogenic strains from the same bacterial species, e.g. in a competitive colonization experiment of a wild-type versus a mutant strain of E. coli. The alternative of performing RNA-seq. on individual strains physically purified prior to RNA isolation—for example by FACS sorting based on fluorescent markers—is complicated by the challenge to maintain the original expression profile of the cells through the procedure of cell harvesting from the intestine, washing, (staining) and cell sorting; in short, the transcriptome of such cells would almost inevitably be technically biased, for example by the rapid response to oxygen exposure. Finally, the recently developed microbial single-cell RNA-sequencing approaches have not yet been tested for their ability to perform genome-scale transcriptomics in complex in vivo environments like the gut. Considering that Record-seq derived sentinel cells are based on the CRISPR spacer acquisition system of Fusicatenibacter saccharivorans (Fs), the inventors hypothesized that the inventors could leverage the differences in the sequences between the two CRISPR arrays in the endogenous CRISPR locus of this bacterium to construct a novel generation of recording constructs enabling parallel transcriptional profiling of two isogenic strains of E. coli. Since a stretch of sequence that is distinct between the DRs of these two CRISPR arrays is maintained throughout the library preparation procedure, this sequence could serve as a barcode and enable us to computationally discriminate spacers acquired into the two CRISPR arrays. The inventors had previously demonstrated that both FsCRISPR array-1 and array-2 were capable of spacer acquisition in an E. coli host. As the inventors expected that the overall E. coli biomass would approximately be distributed amongst the two isogenic strains, and thus the overall data yield would be halved, the inventors set out to improve the efficiency of Record-seq leveraging recent findings showing that CRISPR spacer acquisition is aided by transcription-coupled repair. After testing a panel of constitutive promoters of different strengths upstream of the CRISPR array, the inventors found new constructs that improved CRISPR spacer acquisition efficiency by identifying pFS_1113 as the most efficient construct for spacer acquisition in vitro (FIG. 15B) and in vivo for the DR2-containing array-2 by 3.9-fold compared to pFS_0453 (FIG. 15C). The inventors validated these findings for selected constructs in vivo (FIG. 15C) confirming pFS_1113 as the inventor's lead candidate. The inventors further optimized the library preparation protocol for a parallel readout of both CRISPR arrays from a single reaction to minimize any potential technical biases while extracting transcriptional profiles. The inventors also confirmed that the fraction of spacers erroneously assigned to the wrong barcoded CRISPR array was negligible when performing SENECA on isolated cultures of DR1 or DR2 plasmid with a mixture of adapter ligation oligos compatible to DR1 and DR2 (FIG. 15D). The inventors next investigated the presence of any intrinsic bias in transcriptional records acquired by DR1- or DR2-harboring CRISPR arrays by performing Record-seq in vitro using both DR-barcoded plasmids. While transcriptional records acquired by DR1 and DR2 arrays were well correlated (FIG. 15E), the inventors observed that the DR had an impact on the efficiency of recording with DR2 acquiring spacers more efficiently than DR1 (FIG. 15F). This finding is in line with the inventor's previous observations but had the potential to introduce technical variance into Record-seq libraries of two isogenic strains labelled with either DR1 or DR2 (FIG. 15H). To exclude false positive discoveries of differentially expressed genes introduced by the previously noted differences in DR1 and DR2 recording efficiencies (FIG. 15F), the inventors opted for a full factorial design, colonizing germ-free mice with either (1) MG1655 WT-DR2 in competition with MG1655 ΔuxaC-DR1 or MG1655 WT-DR1 in competition with MG1655 ΔuxaC-DR2.

### Example 10: Extended Conclusion on the Advantages and Limitations of Record-Seq Sentinel Cells

Whereas RNA-seq provides a snapshot of cellular gene expression activity, Record-seq is based on cumulative transcriptional performance throughout a defined time window. In response to an acute environmental stimulus that alters the transcriptional landscape of the cell, the “signature” of the changed environment will be recorded as novel spacers but Record-seq will-unlike RNA-seq-still provide an integral of the spacers acquired before and after the environmental change. Future work will help reveal the extent to which this previous cellular record affects the sensitivity of Record-seq sentinel cells to capture rapid transcriptomic changes and how the performance of Record-seq compares to RNA-seq in this regard. While the current efficiency of transcriptional recording is sufficient to capture complex and dynamic intestinal environments in simple and complex microbiota mice, a more efficient system, or one combined with new functionalities as well as a system independent of the use of a plasmid-encoded selection marker (KanR) for long-term recording, could expand the utility of the approach and open up exciting future avenues. In this work, the inventors improved the efficiency of Record-seq by 39-fold compared to the inventor's previous publication, using insights from single-molecule studies on the CRISPR spacer acquisition process. Further improvements could be achieved using the toolbox of protein or bacterial strain engineering techniques with the goal of optimizing CRISPR-Cas components or the bacterial chassis themselves, respectively. Use of other cellular chassis, such as commensal bacteria colonizing more densely than E. coli or occupying specific niches of the gastrointestinal tract, could potentially be employed alternatively or in combination to provide a richer or targeted picture of intestinal function. In the future, transcriptional recording sentinel cells may also be applied to other microbe-containing environments, including human microbiomes and open environments for applications in biomedicine, environmental monitoring, and agriculture.

### Example 11: Transcriptional Recording in Conventional Microbiota Mouse Models

### Genomically-Integrated Recording Components in Probiotic Bacteria, Record-Seq V2.0

With the final aim of generating an engineered bacterial candidate that could be administered to humans, we aimed to implement the Record-seq technology in a probiotic strain. Specifically, we chose Escherichia coli Nissle 1917 (EcN), which has been employed for more than 100 years for improving human health. Different living biotherapeutic companies are rationally engineering EcN and using these generated strains as therapy against diverse diseases, such as metabolic diseases or cancer. Some of these candidates are advancing through clinical trials, reaching Phase III, and therefore close to their clinical application in patients. All these facts encouraged us to try to switch to EcN as candidate strain for our recording sentinel cells.

Besides the use of this probiotic as “bacterial chassis” for our technology, we also aimed to improve the safety by eliminating antibiotic resistances. The original configuration of the Recording construct (FsRTCas1-Cas2) was constructed in a bacterial plasmid (namely pFS0453 and derivatives), whose maintenance in the bacteria depended on the introduction of a kanamycin resistance cassette, with the implications that this has in a world where bacterial antibiotic resistance is becoming a global health issue. Furthermore, the fact that this resistance is encoded in a plasmid, which is a mobile genetic element, would have added extra risk of spreading the antibiotic resistance gene to other microbiota members. Thus, we tried to solve these challenges by introducing the recording construct in the genome of EcN. By using this approach, we would make sure the recording elements are being replicated together with the bacteria in a stable way without the need of harboring any of these artificial antibiotic resistances. For that, we adapted a conjugation-based genomic integration protocol allowing scarless integration of genetic elements for our purposes. We chose the genomic locus of the flu gene for integrating FsRT-Cas1, Cas2 and the array (Record-seq genetic construct), generating the integrative construct pAAC_0001. The resulting strain after genomic integration of this element was called EcN flu::AAC1.

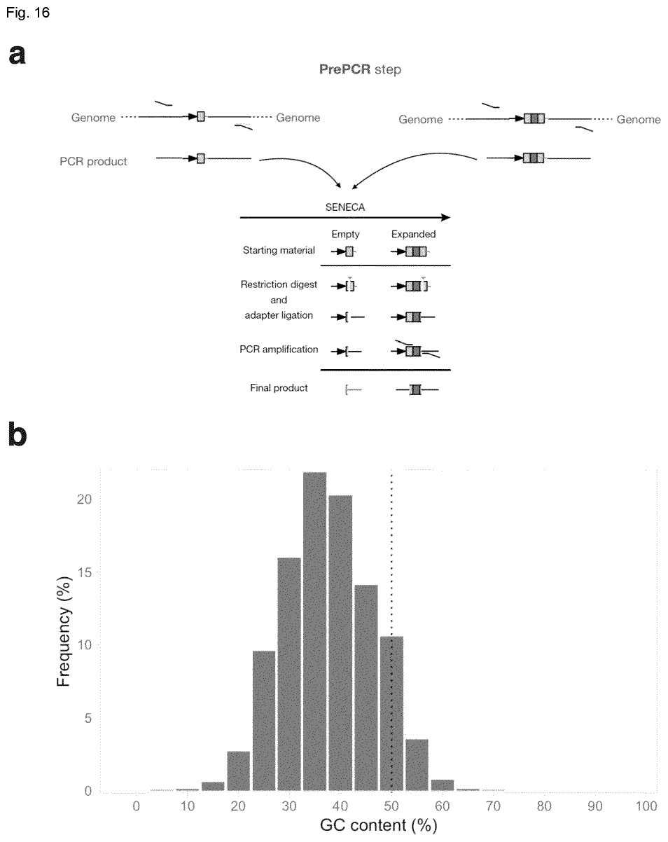

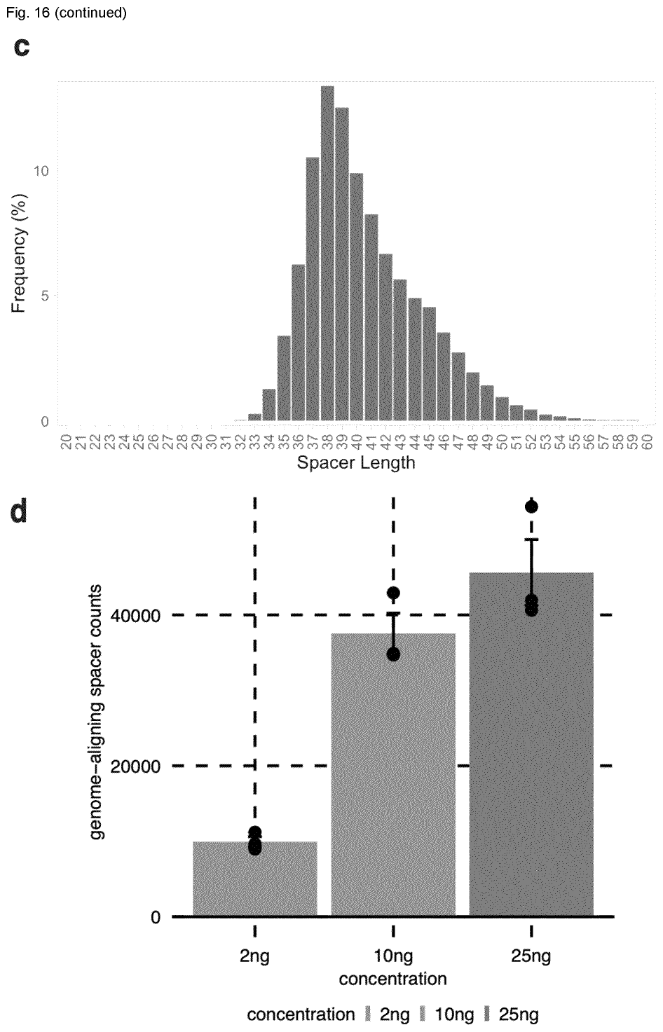

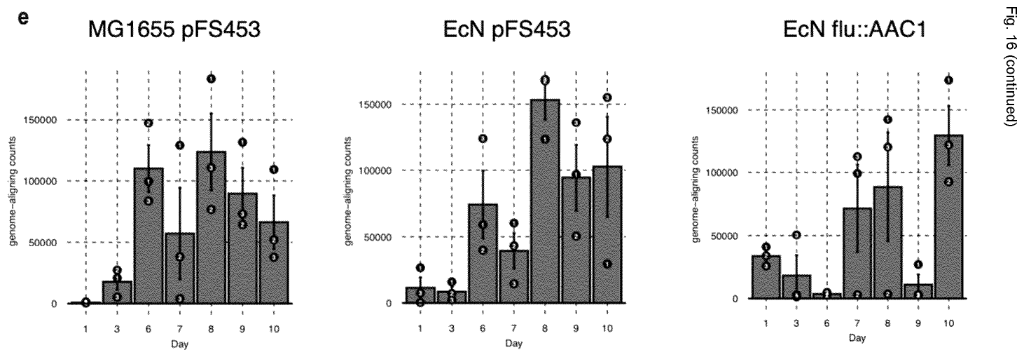

Given that the recording machinery is only encoded in one copy per bacterial cell and in a more complex genetic background compared to a plasmid, this required the creation of a new method to readout acquired spacers. We sought to adapt the Selective Amplification of Expanded Arrays (SENECA) protocol to this new circumstance. This was solved by the ideation and optimization of a pre-PCR step in which the CRISPR array region is selectively amplified from the genome with specifically designed primers (FIG. 16A). The amplification product was then used as input for performing the classical SENECA protocol. After the adoption of this extra step, we could demonstrate spacers were acquired and therefore recording was function as expected using integrated EcN, without the presence of any antibiotic resistance markers (FIG. 16B-D). The analysis of this acquired spacers showed similar characteristics to the one acquired by the previous candidate (E. coli MG1655 harboring pFS453), such as similar GC content and length (FIG. 16B,C). The number of spacers aligning to the genome of EcN was around 750000 (FIG. 16D), demonstrating that we can get a good picture of the transcriptional state of the cell by using the new candidate. These results, in sum, showed Record-seq can be triggered from a probiotic strain without antibiotic resistances in a safe way, and clears the way to use this technology in humans.

### Record-Seq V2.0 Validation In Vivo in Mice

To demonstrate the functionality of our Record-seq V2.0 strain, we performed in vivo recording experiments. The mouse model we employed was monocolonized germ free mice, in which the only bacteria that will be present in the mouse gut will be the one administered. This is the same model Schmidt et al. (Schmidt, Science, 2022) used with the previous plasmid-based strain, with robust recording of differential transcriptional signatures under different diets, inflammation or presence of other bacteria. We gavaged into monocolonized germ free mice the previous strain MG1655 with the recording plasmid (pFS_453), as well as EcN with pFS_453 or integrated into the genome (EcN flu::AAC1). The mice received the inducer aTc in drinking water, and fecal samples were collected at days 1, 3, 6, 7, 8, 9 and 10. After optimization of the SENECA protocol using these samples, we could proceed with extracting the recorded information. We showed that the number of unique spacers acquired by the new strain was similar when compared to the plasmid-based bacteria, with even higher acquisition at later timepoints (FIG. 16E). This constitutes the first evidence that the safe integrated strain can record transcriptional information in vivo in the intestine.

We now aimed to expand the in vivo experiments to complex microbiota mice (specific pathogen free model, SPF), where our bacteria would not be alone in the gastrointestinal tract after gavaging and the transit time would be shorter, increasing the complexity of transcriptional information acquisition. We also wanted to test different gavage methods, specifically intraduodenal gavage and intragastric, and the preinduction of the bacterial culture with aTc prior to gavage. For this purpose, we gavaged EcN flu::AAC at 1e9 CFU/mice and collected fecal samples at 6, 12 and 24 hours. The results of the experiments showed recording is possible with this bacterial candidate in SPF mice (FIG. 16F). This experiment thus demonstrates that EcN harboring Record-seq components function as sentinel cells in unperturbed animals harboring a normal microbiota.

Record-Seq V2.1, Installing “Biocontainment” within Record-Seq V2.0 Chassis

In this section we provide a new dataset demonstrating the functionality of this new chassis (Record-seq V2.0) in vivo in mice.

In parallel to the previous experiments described above, we further engineered the bacterial chassis with safety measures, again going towards the objective of its use in humans. This safety-related measures included the incorporation of auxotrophies as a biocontainment strategy in the bacteria. By implementing these, we make sure the bacteria cannot replicate outside of the laboratory environment, as we make it dependent to supplementation of different elements that auxotroph strains cannot synthetize. The technological challenge when using this strategy was to make sure recording is still taking place in these auxotrophic strains, so after constructing the strains auxotrophic for diaminopimelic acid (by knocking out the gene dapD) and thymidine (thyA knockout) we tested their recording efficiency by quantitative SENECA (qSENECA). The results of these experiments in vitro showed that the auxotrophic strains retained recording capacity at similar efficiency compared to the WT parental strain (FIG. 16G). This thus indicated this biocontainment strategy can be implemented in the final candidate for limiting their growth in unintended environments.

Furthermore, we also deleted one of the genes (clbA) implicated in the biosynthesis of the native colibactin of the EcN strain and the whole pks island, where all the genes synthesizing colibactin are coded. The deletion of these elements respond to the concerns raised by some works showing colibactins can potentially have cytotoxic effects in cultured cells in vitro. The deletion of the clbA gene, as in the case of the auxotrophies, did not reduce recording capacity of the bacteria, showing that this modification can be included in the final candidate (FIG. 16G).

### Capacity of Record-Seq to Function in Presence of Highly Complex Microbiota

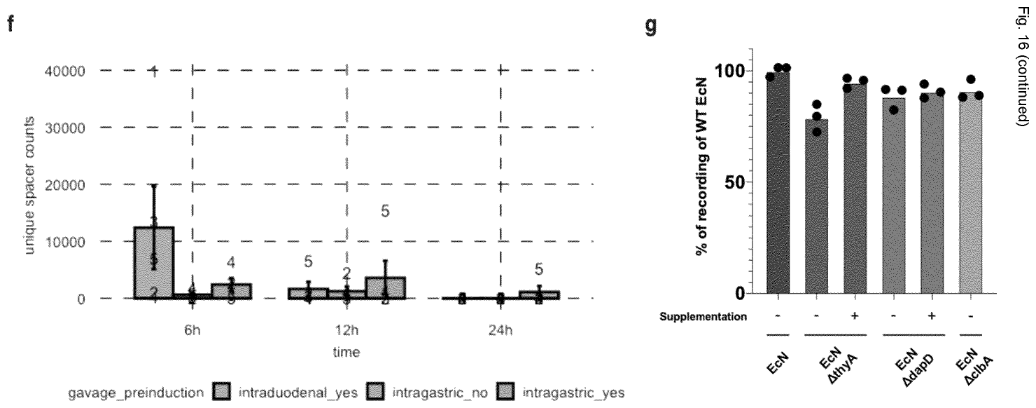

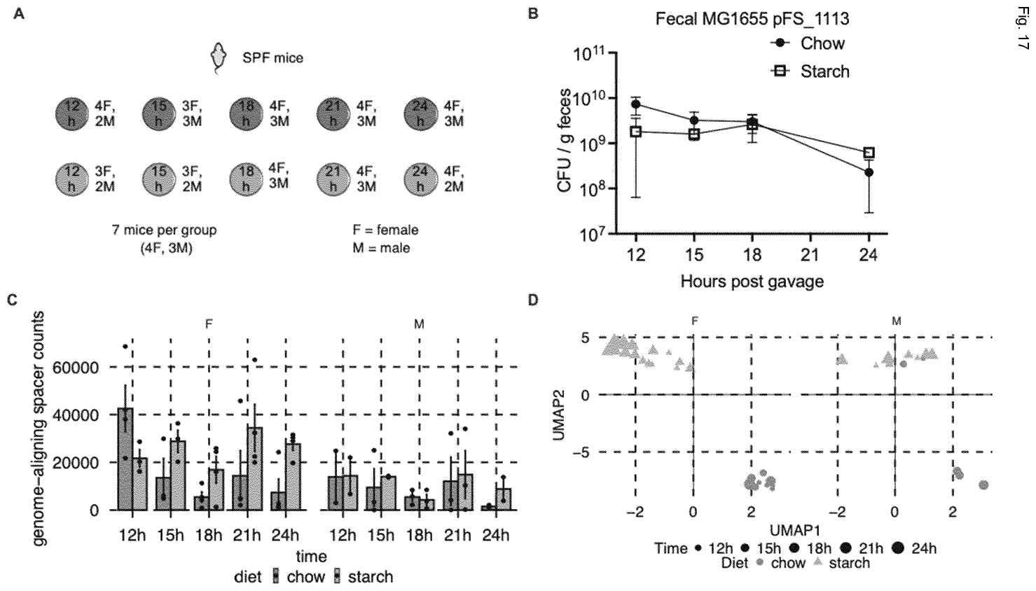

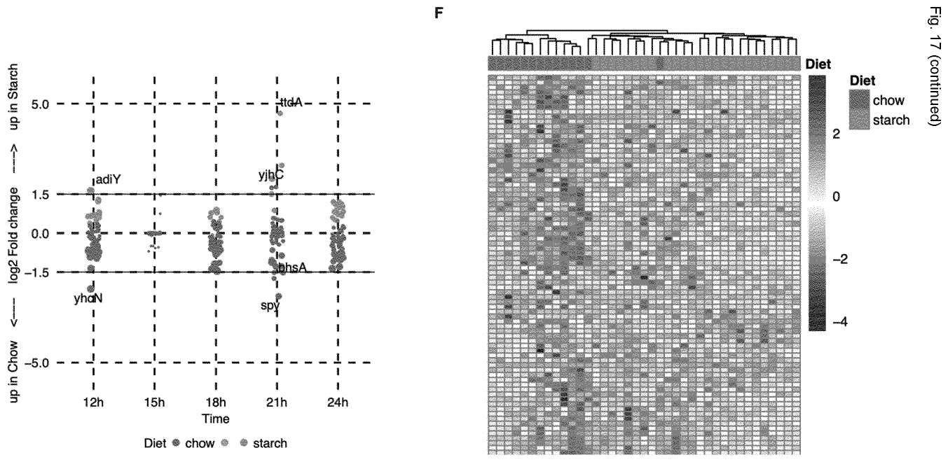

To investigate the capacity of Record-seq to function in the presence of a highly complex microbiota, we gavaged Record-seq sentinel cells encoding pFS_1113 into specific pathogen free mice fed either a standard rodent chow or a starch-based purified diet (FIG. 17A). Although colonization resistance caused the majority of the gavaged E. coli sentinel cells to rapidly pass through the gastrointestinal tract (FIG. 17B), we detected transcriptional records as early as 12 hours after gavage (FIG. 17C). Principal component analysis enabled stratification according to diet group over the course of the 12 h to 24 h sampling time points (FIG. 17D). From 12 hours after gavage onwards, we detected significantly differentially expressed genes (FIG. 17E and F). Among the genes and pathways that were most prominently differentially expressed, we found evidence for increased dgoK/dgoD-dependent galactonate utilization by E. coli in chow-versus starch-fed mice (FIG. 17E). Conversely, under the starch diet, increased recording of yjhC and nanA suggested augmented utilization of host mucus-derived sialic acid in response to a dietary lack of E. coli-accessible carbon and energy sources.

In summary, these data establish that Record-seq can be performed in the context of a complex intestinal microbiota where it yields sufficient numbers of spacers to distinguish diet groups and identify differentially expressed genes that enable conclusions about the luminal conditions. Unlike mice with the sDMDMm2 microbiota that are susceptible to permanent colonization by facultative anaerobic bacteria such as E. coli, SPF mice are resistant to E. coli colonization. The findings presented here demonstrate that Record-seq sentinel cells function in the presence of direct niche competitors such as endogenous Proteobacteria and extend the proven range of usability from sDMDMm2 mice containing just 12 bacterial species to the equivalent of a fully diverse human microbiota.

### Example 12: Conclusions

Here the inventors demonstrate that transcriptional recording sentinel cells using FsRT-Cas1-Cas2 to integrate RNA-derived spacers from the E. coli transcriptome into plasmid DNA-encoded CRISPR arrays are capable of recording complex and dynamic transcriptional changes during E. coli adaptations throughout time, transit, and perturbation of the mammalian intestinal tract. This scalable non-invasive system for assessing intestinal function in vivo archives characteristic microbial signatures of physiological or pathological states. Transcriptome-scale recordings elucidate microbial responses to alterations in the intraluminal environment across nutrition, intestinal inflammation and microbe-microbe interactions. The inventors have illustrated how carbon preferences can be shown with the tool without confounding manipulations and validated new findings of intraluminal microbial adaptation even within variants of a single microbial species under different dietary conditions.

Record-seq offers multiple advantages compared to contemporary techniques. First, unlike conventional cell-based biosensors, Record-seq does not require a specific biosensor for every biomolecule of interest and can report on a wide range of complex biological features-thereby serving an unbiased discovery tool. Second, compared to conventional omics-based technologies run on fecal samples, Record-seq integrates information on gut function along the length of the intestine which is particularly valuable for studying the proximal large intestinal environment that has been largely refractory to detailed studies due to its inaccessible location. Third, multiplexed Record-seq reveals diverse in situ microbe-microbe interactions within the same animal over time, which cannot be readily scaled or implemented in high throughput with conventional methods.

### Example 13: Material and Methods

### Bacterial Strains

Bacterial strains used in this study were Escherichia coli strains MG1655 (ATCC no. 700926) and BL21 (DE3) Gold (Agilent no. 230132) and Bacteroides thetaiotaomicron strain VPI-5482 (ATCC no. 29148). E. coli MG1655 Str^{R}, E. coli MG1655 Str^{R} NaI^{R}, E. coli MG1655 Str^{R} ΔidnK/ΔgntK, and E. coli MG1655 Str^{R} ΔuxaC were provided by T. Conway and the Kan^{R} marker of the ΔuxaC strain was removed using pCP20 recombination as reported previously (K. A. Datsenko et al., Proceedings of the National Academy of Sciences of the United States of America 97, 6640-6645 (2000)) yielding E. coli MG1655 Str^{R} ΔuxaC AKan^{R}. All E. coli strains used in this study are reported in Table 1. MG1655 isolates have been sequenced and their genomic sequences are available in the NCBI Assembly database (PRJNA807125). NCBI Reference Sequences U00096.3 and NC_012947.1 were used for MG1655 and BL21 (DE3) Gold, respectively. The stable defined moderately diverse mouse microbiota 2 (sDMDMm2) has been described previously and is available through the Deutsche Sammlung für Mikroorganismen und Zellkulturen DSMZ. The constituting taxa were originally isolated from the mouse intestine and comprise Bacteroides 148, Blautia YL58, Akkermansia YL44, Bacteroidales YL27, Ruminococcaceae KB18, Lactobacillus 149, Lachnospiraceae YL32, Erysipelotrichaceae 146, Enterococcus KB1, Flavonifractor YL31, Parasutterella YL45 and Bifidobacterium YL2 (Table 2).

TABLE 1
E. coli strains used in this study.

| E. coli strain | supplier | order # | genotype |
|---|---|---|---|
| BL21-Gold(DE3) | Agilent | 230132 | E. coli B F⁻ |
| | Technologies | | ompT hsdS(rB⁻ |
| | | | mB⁻) dcm⁺ |
| | | | Tet^{R} gal λ(DE3) |
| | | | endA Hte |
| MG1655 (Bern) | Andrew | NA | F⁻ lambda⁻ rph-1 |
| | Macpherson | | |
| MG1655 Str^{R} | Tyrrell | NA | F⁻ lambda⁻ |
| ΔgntK/ΔidnK | Conway | | rph-1 ΔgntK |
| | | | ΔidnK Str^{R} |
| MG1655 Str^{R} ΔuxaC | Tyrrell | NA | F⁻ lambda |
| | Conway | | rph-1 ΔuxaC Str^{R} |
| | | | Kan^{R} |
| MG1655 Str^{R} | Tyrrell | NA | F⁻ lambda⁻ |
| ΔuxaC ΔKan^{R} | Conway | | rph-1 ΔuxaC Str^{R} |
| MG1655 Str^{R} | Tyrrell Conway | NA | F⁻ lambda⁻ rph-1 Str^{R} |
| MG1655 Str^{R} Nal^{R} | Tyrrell Conway | NA | F⁻, lambda⁻ rph-1 |
| | | | Str^{R} Nal^{R} |

TABLE 2
Taxa of the stable defined moderately diverse
mouse microbiota 2 (sDMDMm2).

| Bacterial species | DSMZ |
|---|---|
| Lachnoclostridium sp. YL32 | DSM 26114 |
| Ruminiclostridium sp. KB18 | DSM 26090 |
| Bacteroides sp. I48 | DSM 26085 |
| Parabacteroides sp. YL27 | DSM 28989 |
| Burkholderiales bacterium YL45 | DSM 26109 |
| Erysipelotrichaceae bacterium I46 | DSM 26113 |
| Blautia sp. YL58 | DSM 26115 |
| Flavonifractor plautii YL31 | DSM 26117 |
| Bifidobacterium animalis subsp. animalis YL2 | DSM 26074 |
| Lactobacillus reuteri I49 | DSM 32035 |
| Akkermansia muciniphila YL44 | DSM 26127 |
| Enterococcus faecalis KB1 | DSM 32036 |

### Mice

All mouse experiments were performed in accordance with Swiss federal and cantonal regulations under permit numbers BE43/16, BE44/18 and BE107/20. Germ-free C57BL/6 (J) mice were born and housed in flexible-film isolators in the Clean Mouse Facility, University of Bern, Switzerland. Unless noted otherwise, mice received a vitamin-fortified rodent chow diet (Kliba Nafag 3307) sterilized by autoclaving for 20 min at 132° C. and water ad libitum. Age and sex-matched mice were used at 6-15 weeks of age (mostly 8-12 weeks). Mice were constantly and independently confirmed to be germ-free within the breeding isolators by culture-dependent methods (liquid cultures in brain-heart infusion (BHI) broth (Thermo Fisher Scientific) aerobically at 37° C. at 180 rpm and anaerobically in an anaerobic cabinet (Meintrup DWS) containing 80% N₂, 10% H₂ and 10% CO₂ at 37° C. without shaking) and culture-independent methods i.e. microscopic examination of fecal smears stained with the DNA dye SYTOX green (Thermo Fisher Scientific).

During experiments, the absence of bacteria other than E. coli (and B. thetaiotaomicron in experiments related to FIG. 5 and FIG. 13) was constantly confirmed by culturing of fecal suspensions on lysogeny broth (LB) agar aerobically and on BHI agar with 5% defibrinated sheep blood anaerobically. The microbial biomass per gram feces was determined by weighing fecal pellets, homogenizing them in 1 ml of PBS using a Retsch MM400 tissue lyser at 30 Hz for 3 min and streaking serial dilutions in PBS onto LB agar.

One day before gavage, drinking water of the mice was exchanged for water containing 30 μg/ml of anhydrotetracycline (aTc) (Adipogen) and 100 μg/ml of kanamycin sulfate (MP biochemicals) which was prepared by diluting stock solutions of 2 mg/ml of aTc in 95% ethanol and 100 μg/ml of kanamycin sulfate in aqua bidest into sterile tap water.

### Plasmid Transformation

For plasmid transformation, E. coli BL21 (DE3) Gold (Agilent Technologies) and E. coli MG1655 (ATCC no. 700926) were made chemically competent using the Mix & Go E. coli Transformation Kit & Buffer Set (Zymo Research). For this, strains were streaked to single colonies on LB (Difco) agar (Huberlab) plates without antibiotics followed by growth overnight at 37° C. A single colony of E. coli was inoculated into 50 ml of ZymoBroth (Zymo Research) and grown at 19° C., 220 rpm in an orbital shaker (New Brunswick Innova 40R) to an optical density of OD₆₀₀=0.45. Subsequently, cells were made competent following the manufacturers protocol, dispensed to aliquots of 25 μl, flash-frozen in liquid nitrogen, and stored at −80° C.

Transformation with the recording plasmid pFS_0453 (Addgene #117006) was performed by adding ˜60 ng of plasmid DNA to 25 μl of competent cells, followed by heat shock (42° C., 30 s), recovery in 120 μl of S.O.C medium at 37° C., 900 rpm, 30 min and spreading on LB agar plates containing 50 μg/ml of kanamycin sulfate (Biochemica). Glycerol stocks were created by growth of transformants in LB with 50 μg/ml of kanamycin sulfate at 37° C., 180 rpm in bacterial culture tubes followed by mixing of 500 μl of saturated culture with 500 μl of sterile filtered 50% (v/v) glycerol and freezing at −80° C. for long-term storage.

### Oral Gavage

Unless stated otherwise, a saturating gavage dose of 1·10⁹ colony forming units (CFU) of E. coli was used to avoid confounding the biological signal reported by the inventor's sentinel cells with an initial expansion in the gastrointestinal tract. To maintain the recording plasmid and ensure functional stability of the sentinel cells, the inventors added kanamycin sulfate to the drinking water and confirmed that the transformed cells colonized at 7.9±2.6·10⁹ CFU/g feces, comparably to what has been reported for the parental strain. For oral gavage, E. coli MG1655 or BL21 (DE3) each transformed with pFS_0453 was inoculated from freshly grown colonies and cultured overnight under aerobic conditions in LB containing 50 μg/ml of kanamycin sulfate at 37° C., 180 rpm. Upon saturation, cultures were centrifuged at 3,480 g for 10 min at room temperature and washed twice with the equivalent volume as the LB culture in sterile phosphate-buffered saline (PBS) (8 g per liter of NaCl, 0.2 g per liter of KCl, 1.44 g per liter of Na₂HPO₄, 0.24 g per liter of KH₂PO₄, all from Sigma-Aldrich). The required dose of bacteria was resuspended in PBS (1·10⁹ CFU per 500 μl). The bacterial suspension was orally gavaged directly into the mouse duodenum with a 12 gauge straight stainless-steel needle (Provet AG) attached to a 2-ml syringe. The culture volume was chosen according to the number of mice to be gavaged with the equivalent of 3 ml of saturated culture being gavaged into each mouse. Culture vessels were at least twice as big as the culture volume to ensure proper aeration. For example, to gavage n=15 mice, E. coli was grown in 100 ml of LB broth in a 250-ml bottle, washed twice with 100 ml of PBS and resuspended in 16.6 ml of PBS from which 500 μl were gavaged into each mouse. Gavage doses and absence of contamination were confirmed by streaking serially diluted suspensions onto LB agar.

### Isolation of RNA and RNA-Seq

Fecal pellets were homogenized in 1 ml of PBS using a Retsch MM400 tissue lyser at 30 Hz for 3 min. Large particles were pelleted by centrifugation at 200 g for 2 min at room temperature. Bacteria in the supernatant were pelleted by centrifugation at 6,800 g for 3 min at room temperature and lysed in 100 μl of RNA extraction solution containing 95% (v/v) formamide (VWR), 0.025% (w/v) SDS (Bio-Rad), 18 mM EDTA (Merck Millipore), 1% 2-mercaptoethanol (Merck Millipore) at 95° C., 1,200 rpm shaking for 7 min. Following centrifugation at 16,000 g for 5 min at room temperature, the RNA in the supernatant was purified with the RNeasy clean-up kit (QIAGEN) following the manufacturer's instructions, including the optional DNase treatment (15 min at room temperature). RNA was frozen at −80° C. for storage and submitted to the Next Generation Sequencing (NGS) Platform Bern for ribosomal RNA (rRNA) depletion using the RiboMinus Transcriptome Isolation Kit, bacteria (Invitrogen), followed by library preparation using the Illumina TruSeq Stranded total RNA kit (Illumina) and sequencing on an Illumina NovaSeq platform using the NovaSeq 6000 SP Reagent Kit (100 cycles).

Isolation of Plasmid DNA from Feces and Intestinal Contents.

For E. coli monocolonized mice, 50-100 mg of fecal material or intestinal contents were collected and frozen at −20° C. After thawing, feces or intestinal contents were homogenized in 1 ml of PBS using a Retsch MM400 tissue lyser at 30 Hz for 3 min. Large particles were pelleted by centrifugation at 200 g for 2 min at room temperature. Bacteria in the supernatant were pelleted by centrifugation at 6,800 g for 3 min at room temperature and plasmid DNA was isolated using the QIAprep spin Miniprep kit (QIAGEN) with the following modifications: E. coli cells were resuspended in 500 μl of buffer P1, lysed by the addition of 500 μl of buffer P2 for 5 min at room temperature. The reaction was neutralized by the addition of 700 μl of buffer N3 and centrifuged at 18,000 g for 10 min at room temperature to pellet debris. Supernatant was passed through a spin column on a vacuum manifold (QIAGEN), upon which the column was washed with 500 μl of buffer PB followed by 700 μl of buffer PE. Residual wash buffer was removed by centrifugation at 18,000 g for 1 min at room temperature. For cecal contents of monocolonized mice (˜200 mg), buffer volumes were increased to 1000 μl of buffer P1, 1000 μl of buffer P2 and 1400 μl of buffer N3, whereas volumes of buffer PB and PE remained the same. Plasmid DNA was eluted from the column by the addition of 50 μl of pre-warmed buffer EB (55° C.) followed by incubation at 55° C. at 850 rpm for 3 min followed by centrifugation at 10,000 g for 1 min. A total of three elution steps with 50 μl were performed yielding 150 μl of eluate. DNA from this eluate was precipitated by the addition of 15 μl of 3 M sodium acetate solution (Sigma-Aldrich) and 150 μl of 2-propanol (Merck-Millipore) followed by incubation at −20° C. for 20 min and centrifugation at 20,000 g for 20 min at room temperature. Supernatant was carefully removed without disturbing the pellet which was washed with 500 μl of 80% (v/v) ethanol and centrifuged at 20,000 g for 15 min at room temperature. Ethanol was removed completely and the pellet was briefly dried (55° C., 30 sec) before resuspension in 15 μl of buffer EB and transfer to 96-well PCR plates for storage at −20° C. or immediate use in SENECA.

Quantification of Isolated Plasmid DNA by Droplet Digital PCR (ddPCR)

For quantification by ddPCR, plasmid DNA was first diluted 100,000-fold in ddPCR dilution buffer. ddPCR dilution buffer consists of 2 ng/μl of sheared salmon sperm DNA (Sigma Aldrich), 0.05% Pluronic F-68 (Invitrogen) in UltraPure DNase/RNase-Free distilled water (Thermo Fisher Scientific). Dilution steps were carried out in twin.tec PCR plate 96 LoBind (Eppendorf) in the following steps: dilution 1:1 μl of plasmid DNA, 49 μl of ddPCR dilution buffer, followed by dilution 2:1 μl of dilution 1, 49 μl of ddPCR dilution buffer and finally dilution 3:1 μl of dilution 2, 39 μl of ddPCR dilution buffer. Primer-probe assays targeting FsRT-Cas1 were prepared by mixing of 180 μl of FS_2814 (100 UM), 180 μl FS_2815 (100 μM), 50 μl of FS_2816 (100 UM) with 590 μl of TE buffer (Sigma Aldrich) (Table 3). Aliquots of 100 μl of primer-probe assay were prepared and stored in 1.5 ml amber tubes (Eppendorf) at −20° C. ddPCR was performed by mixing 4.5 μl of dilution 3 template, 1.1 μl of primer-probe assay, 0.25 μl of FastDigest XhoI (Thermo Fisher Scientific), 11 μl of ddPCR Supermix for probes (no dUTP) (Bio-Rad) and 5.4 μl of UltraPure DNase/RNase-Free distilled water per reaction. PCR reactions were dispensed into droplets using the QX100 droplet generator (Bio-Rad) according to the manufacturer's protocol. PCR amplification was performed in an Eppendorf Mastercycler Gradient (95° C. for 10 min, followed by 42 cycles of 95° C. for 30 s, 57.1° C. for 60 s, 72° C. for 15 s final extension and a final 98° C. 10 min step) with a ramp rate for all steps of 2° C./s as specified by the manufacturer. Readout was performed using the QX100 droplet reader (Bio-Rad), cut-off for positive droplets was manually set to 3,500.

TABLE 3
ddPCR primers and probe.

| Primer | Sequence (5′ → 3′) | SEQ ID NO |
|---|---|---|
| FS_2814 | GTACTGGCGTATGAATCACG | 70 |
| FS_2815 | CGAATCAGGATAATACCCGG | 71 |
| FS_2816 | HEX-AGCGATCTGAAGAACC | 72 |
| | AGGAAT-BHQ-1 | |

### Selective Amplification of Expanded CRISPR Arrays (SENECA)

The SENECA library preparation method has been extensively described before (T. Tanna et al., Nature protocols 15, 513-539 (2020)). All DNA oligonucleotides were ordered from IDT (Table 4), FastDigest FaqI (ThermoFisher Scientific), T7 DNA Ligase and NEBNext High Fidelity PCR Master Mix, 2× (both New England Biolabs). Due to the low concentration of plasmid DNA extracted from fecal pellets and residual genomic DNA, input DNA was not normalized but instead 3.75 to 7.5 μl of purified plasmid DNA was used for SENECA adapter ligation, volumes are stated in the subsections below corresponding to the respective experiments along with the specific annealed oligonucleotides (carrying library barcodes) for adapter ligation. SENECA first round PCR was performed with 23 cycles instead of 22 cycles and otherwise as described before (T. Tanna et al., 2020, ibid). After second round PCR, 3 μl of each sample were mixed with 17 μl of UltraPure DNase/RNase-free distilled water (Thermo Fisher Scientific) and loaded on an E-Gel 48 Agarose Gel, 2% along 150 ng of GeneRuler low-range DNA ladder (Thermo Fisher Scientific) in 20 μl for gel-based quantification using the software Bio-Rad Image Lab version 6.0.1. Samples from an experiment were assigned to 5 bins based on their DNA concentrations and pooled according to these bins. Each sub-pool was purified separately by PCR purification and gel extraction from E-GelEX 2% (Thermo Fisher Scientific) agarose gels as described previously, individually quantified by quantitative PCR (qPCR) using the KAPA Library Quantification Kit for Illumina Platforms (Roche), pooled to achieve equal sequencing depth according to the number of samples in each sub-pool and sequenced on an Illumina NextSeq 500/550 platform as described before. As Record-seq is a population-based measurement requiring many cells to reconstruct a cellular history, and in vivo experiments present a material-limiting environment, the inventors addressed the technical inputs and outputs of the inventor's workflow (FIG. 7A and B). Record-seq usually used an input, of approximately 4.7±0.7·10⁸ cells per sample (3-4 fecal pellets of ˜27 mg each per mouse with a biomass of 5.6·10⁹ sentinel cells per gram). After 24 hours of in vivo recording, the Record-seq output was 3.6±0.6·10³ spacers, which increased by 1.0±0.5·10⁴ spacers per day. After 7 days of recording, the inventors found 1.2±0.1·10⁵ spacers, which aligned to 91±1% of the 4419 transcripts in the E. coli transcriptome.

TABLE 4
Olignonucleotides for cloning and SENECA adapter
ligation oligonucleotides.

| Primer | Sequence (5′ → 3′) | SEQ ID NO |
|---|---|---|
| FS_0963 | GTGACTGGAGTTCAGACGTGTGCTCTTCCGATC | 14 |
| FS_0964 | AAAGGATCGGAAGAGCACACGTCTGAACTCCAGTCAC | 15 |
| FS_2759 | AAAGATTTGTACCAAGGTTCCTAGNNNNNNNNNNGATCGGAAGA | 16 |
| | GCACACGTCTGAACTCCAGTCAC | |
| FS_2769 | CTAGGAACCTTGGTACAAAT | 17 |
| FS_3046 | GAGTTGATAGACAATGTAACCCACTCGTGCACCTCGAGCAACTGA | 18 |
| | TCTTATAGATACAGCATCTTTTACTTTCCTCGAGTAGCCTAGCAT | |
| | AACCCCGCGGGGCCTCTTCGGGGGTCTCGCGGGGTTTTTTGCTAT | |
| | AAAACGAAAGGCTCAGTCGAAAGACTGGGCCTTTCGTTTTATCTG | |
| | CTAACAAAGCCCGAAAGGAAGCTGAGTTGGCTGCTGCCACCGCTG | |
| | AGCAATAACTAGCATAACCCCTTGGGGCCTCTAAACGGGTCTTGA | |
| | GGGGTTTTTTGCTGAAAGGAGGAACTATATCCGGATGTCTTCATG | |
| | GTAGTACCAAGATACGAAGACATAGTGGCGGGGAAGCTTATGTTC | |
| | CATAGCAAAAAGTCGGTCAGTCTCGTGGCTGAAATCATGAGTTCC | |
| | ACAAAATGGCTGAAATTCAAGGAAAATCAGGAATCTCAGAAAAAC | |
| | GATCGACCGACTTTTTCGATAAAATGGTTGCAAAAATGAGAAAAA | |
| | TCTGATTTAATAGAATCTGAAAACAGCGGAAATGCTGTTGTCGTA | |
| | CTTTACCTAAAAGGAATTGAAACGTCCCCGCCAGGTTGAATCCGA | |
| | TATTTGGAGGTACGATGGAACAGTCTGGGTGGGATTGAGAAGAGA | |
| | AAAGAAAACCGCCGATCCTGTCCACCGCATTACTGCAAGGTAGTG | |
| | GACAAGACCGGCGGTCTTAAGTTTTTTGGCTGAAGCGGCCGCCTC | |
| | ATGGTTATGGCAGCACTGCATAATTTTCTTA | |
| FS_3047 | CCGGAACTTGACAATTAATCATCCGGCTCGTATAATGTGTGGAG | 19 |
| | G | |
| FS_3048 | CACTCCTCCACACATTATACGAGCCGGATGATTAATTGTCAAGTT | 20 |
| FS_3049 | CCGGATTGACGGCTAGCTCAGTCCTAGGTACAGTGCTAGCTCTA | 21 |
| | GT | |
| FS_3050 | CACTACTAGAGCTAGCACTGTACCTAGGACTGAGCTAGCCGTCA | 22 |
| | AT | |
| FS_3051 | CCGGATTTACGGCTAGCTCAGTCCTAGGTATAGTGCTAGCTCTA | 23 |
| | GT | |
| FS_3052 | CACTACTAGAGCTAGCACTATACCTAGGACTGAGCTAGCCGTAA | 24 |
| | AT | |
| FS_3053 | CCGGATTTACGGCTAGCTCAGTCCTAGGTACAATGCTAGCTCTA | 25 |
| | GT | |
| FS_3054 | CACTACTAGAGCTAGCATTGTACCTAGGACTGAGCTAGCCGTAA | 26 |
| | AT | |
| FS_3055 | CCGGATTTATAGCTAGCTCAGCCCTTGGTACAATGCTAGCTCTA | 27 |
| | GT | |
| FS_3056 | CACTACTAGAGCTAGCATTGTACCAAGGGCTGAGCTAGCTATAA | 28 |
| | AT | |
| FS_3057 | CCGGATTGACAGCTAGCTCAGTCCTAGGGATTGTGCTAGCTCTA | 29 |
| | GT | |
| FS_3058 | CACTACTAGAGCTAGCACAATCCCTAGGACTGAGCTAGCTGTCA | 30 |
| | AT | |
| FS_3210 | CCGGATTTACAGCTAGCTCAGTCCTAGGGACTGTGCTAGCTCTA | 31 |
| | GT | |
| FS_3211 | CACTACTAGAGCTAGCACAGTCCCTAGGACTGAGCTAGCTGTAA | 32 |
| | AT | |
| FS_3212 | CCGGACTGATAGCTAGCTCAGTCCTAGGGATTATGCTAGCTCTA | 33 |
| | GT | |
| FS_3213 | CACTACTAGAGCTAGCATAATCCCTAGGACTGAGCTAGCTATCA | 34 |
| | GT | |
| FS_3214 | CCGGACTGATAGCTAGCTCAGTCCTAGGGATTATGCTAGCTCTA | 35 |
| | GT | |
| FS_3215 | CACTACTAGAGCTAGCATAATCCCTAGGACTGAGCTAGCTATCA | 36 |
| | GT | |
| FS_3344 | GTGATCTAACTCGAGTAGCCTAGCATAACCCCGCGGGGCCTCTT | 37 |
| | CGGGGGTCTCGCGGGGTTTTTTGCTATAAAACGAAAGGCTCAGT | |
| | CGAAAGACTGGGCCTTTCGTTTTATCTGCTAACAAAGCCCGAAA | |
| | GGAAGCTGAGTTGGCTGCTGCCACCGCTGAGCAATAACTAGCA | |
| | TAACCCCTTGGGGCCTCTAAACGGGTCTTGAGGGGTTTTTTGCT | |
| | GAAAGGAGGAACTATATCCGGACTGATAGCTAGCTCAGTCCTAG | |
| | GGATTATGCTAGCTCTAGTAGTGGAGAATTAAATTGGAAAAAGT | |
| | CGGTCGATCTCATGCCTGAAATCATGAATTCCGCAAAATGGCGG | |
| | AAATTTAAGGAAAATCAGGAATCTCAGAAAAACGATCGACCGAC | |
| | TTTTGTGATAAAATGGTTGCAAAAAAGAGAAAAATTTGATTTAA | |
| | TAGAATGTGAAAATAGCGGAAATGCTGATGTTGTACCTTACCTA | |
| | TGAGGAATTGAAACGTCCCCGCCAGGTTGAATCCGATATTTGGA | |
| | GGTACGATGGAACAGTCTGGGTGGGATTGAGAAGAGAAAAGAAA | |
| | ACCGCCGATCCTGTCCACCGCATTACTGCAAGGTAGTGGACAAG | |
| | ACCGGCGGTCTTAAGTTTTTTGGCTGAAGCGGCCGCTATTCT | |
| FS_3194 | AAAGCTAATATACCACCAGCAGTANNNNNNNNNNGATCGGAAGA | 38 |
| | GCACACGTCTGAACTCCAGTCAC | |
| FS_3204 | TACTGCTGGTGGTATATTAG | 39 |
| FS_3316 | TGAGATTACGATCGCCAGGTCATGNNNNNNNNNNGATCGGAAG | 40 |
| | AGCACACGTCTGAACTCCAGTCAC | |
| FS_3321 | CATGACCTGGCGATCGTAAT | 41 |
| FS_0968 | ACACTCTTTCCCTACACGACGCTCTTCCGATCTNNCCTAAAAGG | 42 |
| | AATTGAAAC | |
| FS_0969 | ACACTCTTTCCCTACACGACGCTCTTCCGATCTNNNCCTAAAAG | 43 |
| | GAATTGAAAC | |
| FS_0970 | ACACTCTTTCCCTACACGACGCTCTTCCGATCTNNNNCCTAAAA | 44 |
| | GGAATTGAAAC | |
| FS_0971 | ACACTCTTTCCCTACACGACGCTCTTCCGATCTNNNNNCCTAAA | 45 |
| | AGGAATTGAAAC | |
| FS_0972 | ACACTCTTTCCCTACACGACGCTCTTCCGATCTNNNNNNCCTAA | 46 |
| | AAGGAATTGAAAC | |
| FS_0973 | ACACTCTTTCCCTACACGACGCTCTTCCGATCTNNNNNNNCCTA | 47 |
| | AAAGGAATTGAAAC | |
| FS_0974 | ACACTCTTTCCCTACACGACGCTCTTCCGATCTNNNNNNNNCCT | 48 |
| | AAAAGGAATTGAAAC | |
| FS_3325 | ACACTCTTTCCCTACACGACGCTCTTCCGATCTNNACCTATGAG | 49 |
| | GAATTGAAAC | |
| FS_3326 | ACACTCTTTCCCTACACGACGCTCTTCCGATCTNNNACCTATGA | 50 |
| | GGAATTGAAAC | |
| FS_3327 | ACACTCTTTCCCTACACGACGCTCTTCCGATCTNNNNACCTATG | 51 |
| | AGGAATTGAAAC | |
| FS_3328 | ACACTCTTTCCCTACACGACGCTCTTCCGATCTNNNNNACCTAT | 52 |
| | GAGGAATTGAAAC | |
| FS_3329 | ACACTCTTTCCCTACACGACGCTCTTCCGATCTNNNNNNACCTA | 53 |
| | TGAGGAATTGAAAC | |
| FS_3330 | ACACTCTTTCCCTACACGACGCTCTTCCGATCTNNNNNNNACCT | 54 |
| | ATGAGGAATTGAAAC | |
| FS_3331 | ACACTCTTTCCCTACACGACGCTCTTCCGATCTNNNNNNNNACC | 55 |
| | TATGAGGAATTGAAAC | |
| FS_2238 | AAAGCACTTTGGTTATAGAAGAGGGATCGGAAGAGCACACGTCT | 56 |
| | GAACTCCAGTCAC | |
| FS_2240 | AAAGTCCCATGAATGTTCCACATGATCGGAAGAGCACACGTCTG | 57 |
| | AACTCCAGTCAC | |
| FS_2246 | GTGACTGGAGTTCAGACGTGTGCTCTTCCGATCCCTCTTCTATA | 58 |
| | ACCAAAGTG | |
| FS_2248 | GTGACTGGAGTTCAGACGTGTGCTCTTCCGATCATGTGGAACAT | 59 |
| | TCATGGGA | |
| FS_2758 | AAAGACTTTCCGCACAAACCGTGANNNNNNNNNNGATCGGAAG | 60 |
| | AGCACACGTCTGAACTCCAGTCAC | |
| FS_2762 | AAAGACAATCCGTCAAGTCACTAGNNNNNNNNNNGATCGGAAG | 61 |
| | AGCACACGTCTGAACTCCAGTCAC | |
| FS_2760 | AAAGTAAACGACTACACCCGCTCGNNNNNNNNNNGATCGGAAG | 62 |
| | AGCACACGTCTGAACTCCAGTCAC | |
| FS_2761 | AAAGCGATATCATCGTCCCTTTGTNNNNNNNNNNGATCGGAAGA | 63 |
| | GCACACGTCTGAACTCCAGTCAC | |
| FS_2768 | TCACGGTTTGTGCGGAAAGT | 64 |
| FS_2770 | CGAGCGGGTGTAGTCGTTTA | 65 |
| FS_2771 | ACAAAGGGACGATGATATCG | 66 |
| FS_2772 | CTAGTGACTTGACGGATTGT | 67 |
| FS_2806 | AAAGACGCAGGAAACAGGCTTGAT | 68 |
| FS_2807 | ATCAAGCCTGTTTCCTGCGT | 69 |

### Primary Analysis of Data

The single-end sequencing readout from Record-seq and RNA-seq was processed and analyzed using a two-stage computational pipeline as described before (T. Tanna et al., 2020, ibid). The first step in the primary analysis pipeline involved pre-processing of sequencing reads using FastQC v0.11.4 (http://www.bioinformatics.babraham.ac.uk/projects/fastqc/) and trimmomatic v0.35 (A. M. Bolger et al., Bioinformatics 30, 2114-2120 (2014)). For Record-seq, FASTQ files containing sequencing results were converted to FASTA files using the FASTX-toolkit v0.0.14 (http://hannonlab.cshl.edu/fastx_toolkit). Reads without the library barcode were excluded and acquired unique spacer sequences were identified from the remaining reads using a dedicated python script which incorporates fuzzy string matching (spacerExtractor.py). Identified unique spacer sequences for Record-seq and sequencing reads for RNA-seq were aligned to merged references that contain E. coli genome and plasmid sequences and annotation using Bowtie 2 (B. Langmead et al., Nature methods 9, 357-359 (2012)). The following E. coli reference genomes were used: E. coli str. K-12 substr. MG1655 (GenBank U00096.3) and E. coli BL21-Gold (DE3) pLysS AG (Ensembl ASM2366v1). Alignments were then processed and assigned to either the reference E. coli genome or plasmid using Samtools v1.3 (H. Li et al., Bioinformatics (Oxford, England) 25, 2078-2079 (2009)), and for Record-seq, duplicate alignments were removed using a custom python script (SErmdup.py). This two-step, stringent filter for duplicate spacers was incorporated to remove multiple instances of the same spacer arising due to amplification or plasmid replication, and thus obtain a conservative estimate of spacer diversity. Count matrices were generated by quantifying alignments using featureCounts from the Subread package (Y. Liao et al., Bioinformatics (Oxford, England) 30, 923-930 (2014)). These count matrices contain transcript counts for each RNA-seq sample and transcript-aligning spacer counts for each Record-seq sample. All the steps of this primary analysis pipeline were implemented in Snakemake (J. Koster et al., Bioinformatics (Oxford, England) 28, 2520-2522 (2012)) workflows.

### Secondary Analysis of Data

Secondary analysis was performed on generated count matrices by building upon the previously described recoRdseq package implemented in R (T. Tanna et al., 2020, ibid). This broadly involved unsupervised clustering of samples and identification of classifier genes based on differential expression analysis. In general, the first step involved filtering count matrices by excluding outlier samples with low cumulative counts (C=Σₜ x_{t,s}; x=count, t=transcript, s=sample) among replicates using an empirically adjusted absolute cumulative counts threshold, as well as a combined threshold for outliers as described below:
(i) include replicate if modified Z-score Zᵢ>−3

$$( {Z}_{i} = \frac{0.675 * ( {y}_{i} - \tilde{y} )}{median ( \vert {y}_{i} - \tilde{y} \vert )} ,$$

where yᵢ=cumulative count for replicate i, \tilde{y}=median cumulative count for all replicates), else
(ii) if Zᵢ<−3, include replicate if relative deviation from the mean of replicates

$${D}_{i} < 0.25 ( {D}_{i} = \vert \frac{{y}_{i} - \overline{y}}{\overline{y}} \vert ,$$

where yᵢ=cumulative count for replicate i, and y̅=mean cumulative count for all replicates).

Lowly abundant (or recorded) transcripts, defined as transcripts having a low cumulative count across samples (Σₛ x_{t,s}; x=count, t=transcript, s=sample), were also excluded from the analysis. Further, the first day after gavage (Day 1) generally yielded low spacer counts and noisy data compared to later days in multi-day experiments, hence Day 1 was excluded from all subsequent analyses. The count matrices were then normalized and transformed using the variance-scaling transformation (VST) implemented in the DESeq2 package (M. I. Love et al., Genome Biol 15, 550 (2014)), which rendered the data approximately homoscedastic. For dimensionality reduction and unsupervised cluster discovery, principal component analysis (PCA) using the R base stats package and Uniform Manifold Approximation and Projection (UMAP) (L. McInnes et al., 2018) using the umap package implemented in R were performed on the vst-transformed count matrices. UMAP parameters and hyperparameters were tuned for each dataset to achieve optimal separation between experimental groups. k-medoids clustering was used to detect clusters in PCA-transformed data. Fixed random number seeds were used to ensure reproducibility of clustering algorithms. PCA and UMAP results as well as other illustrative plots were plotted using the ggplot2 package in R. Differential expression analysis was performed using the Wald test (pairwise comparisons) or likelihood-ratio test (multiple-group comparisons) implemented in DESeq2 and the quasi-likelihood F-test implemented in the edgeR package in R. Differentially expressed genes (DEGs) were defined as the intersect of significant genes (p_{adj}<0.1, where p_{adj}=Benjamini-Hochberg adjusted P-value detected by these two tools). For time-course datasets, DEGs were independently identified for each timepoint and combined differentially expressed gene lists, ordered by the number of independent timepoints each individual gene was detected on, were generated for downstream pathway analysis. Volcano plots were generated for individual timepoints using the log₂ fold change (log₂ FC) and p_{adj} values calculated by DESeq2. For time-course analysis, log₂ FC for all genes in the combined differentially expressed gene lists were plotted over time, with point size indicating p_{adj} values. For downstream pathway analyses, the log₂FC for each gene (and each comparison) was defined as the maximum log₂FC detected for that gene over the time-course. Hierarchical clustering was performed on vst-transformed counts of DEGs after z-score standardization for each gene, and heatmaps were generated using the pheatmap package in R.

EcoCyc pathway enrichment analysis was performed using the Fisher Exact test for lists of DEGs generated for each experiment, and pathway enrichment plots were created using the top hits. Further, network analysis was performed using the StringApp package (N. T. Doncheva et al., J Proteome Res 18, 623-632 (2019)) in Cytoscape with a confidence score>=0.4 for these DEGs. MCL clustering using the clusterMaker2 package (J. H. Morris et al., BMC bioinformatics 12, 436 (2011)) was used to generate gene clusters within gene networks detected using StringApp analysis in Cytoscape, and the functional enrichment function was used to annotate these clusters using KEGG pathways, UniProt keywords, NetworkNeighborAL, GO Process, GO Function, GO Component. Nodes contributing to functional enrichment of a cluster were marked by bold font. The size of a node corresponding to each gene in the STRING networks was adjusted to reflect the detected log₂ FC value for that gene. Overrepresentation analysis (OA) was performed for differentially expressed gene lists from each experiment based on both the Gene Ontology (GO) resource (C. Gene Ontology, Nucleic acids research 49, D325-D334 (2021)) and Kyoto Encyclopedia of Genes and Genomes (KEGG) (M. Kanehisa, et al., Nucleic acids research 49, D545-D551 (2021)) using the clusterProfiler package (G. Yu et al., OMICS 16, 284-287 (2012)) in R. Wherever experiments were replicated, regression analysis was performed to examine the similarity of regulation in the latter experiment for DEGs detected in the initial experiment. The specific analysis parameters used for each experiment and any changes or additions to the workflow are reported in the following sections.

### Titration of aTc Concentration in Drinking Water

Germ-free C57BL/6 (J) mice were maintained on the chow diet as described above and received water containing 1, 10 or 30 μg/ml of aTc as well as 100 μg/ml of kanamycin sulfate one day prior to gavage of 1·10⁹ cells of E. coli BL21 (DE3) transformed with pFS_0453. Plasmid DNA was extracted and concentrated as described above and 6.25 μl of plasmid DNA were used as an input into SENECA using annealed adapter ligation oligonucleotides FS_0963 and FS_0964 (Table 4).

### Record-Seq Comparison of Transient Chow, Fat, and Starch-Based Dietary Stimulus

Germ-free C57BL/6 (J) mice received the standard chow diet (Kliba Nafag 3307) prior to the experiment. With the beginning of the experiment-two days before gavage-one group remained on the chow diet, while the other two groups received either a starch-based purified diet (Research Diets D12450Jii) or a fat-based (lard-based) purified diet (Research Diets D12492ii), both of which were sterilized by two rounds of irradiation with each 10-20 kGy. Mice received drinking water containing 30 μg/ml of aTc and 100 μg/ml of kanamycin sulfate one day before gavage with 1·10⁹ cells of E. coli MG1655 transformed with pFS_0453 as described above. After 7 days on different diets, mice from all groups received the chow diet (effectively switching fat- and starch-fed mice to the chow diet). Plasmid DNA from fecal pellets was extracted and concentrated as outlined above. Additionally, intestinal contents were sampled yielding the data presented in FIG. 7. The initial, 20-day diet switch experiment described in FIG. 1 and FIG. 8 used 3.75 μl of plasmid DNA as an input into SENECA using annealed adapter ligation oligonucleotides FS_2759 and FS_2769 (Table 4). The consecutive, 14-day diet switch experiment described in FIG. 9 used 7.5 μl of plasmid DNA as an input into SENECA using annealed adapter ligation oligonucleotides FS_2240 and FS_2248 (Table 4).

Primary analysis was performed for Record-seq and RNA-seq readout as described above for both experiments. During secondary analysis, the minimum value of cumulative transcript-aligning spacer counts (C) was set to 10,000 for Record-seq samples, and the minimum value of transcript-aligning counts for RNA-seq samples was set to 100,000. PCAs and UMAPs were generated for the top 500 most variable genes across days and diets. For the initial 20-day experiment, hierarchical clustering and heatmap generation were performed for DEGs detected by multiple testing on day 7, since this was the last day when the mice were fed different diets prior to switching all mice to the chow diet. Further, for both experiments, diet-specific signature genes were defined as the top 500 DEGs detected for day 7.

Hierarchical clustering and heatmap generation were performed for the final day in each experiment using these diet-specific signature genes. For the initial experiment, genes enriched or depleted in each diet pair comparison were identified on day 7 using pairwise DE testing and used for generating volcano plots. EcoCyc pathway enrichment was performed using DEGs identified for each diet pair and the top hits were used for creating pathway enrichment plots. STRING network analysis was performed using Cytoscape using DEG with log₂-FC≥1.0. The granularity parameter for MCL clustering of STRING networks was set at 2.5 and stringdb::score was used as array source with an edge cutoff of 0.5. Clusters containing more than 4 nodes were reported. KEGG- and GO-based OA were performed for DEGs identified for each diet pair using clusterProfiler. Regression analysis was performed to examine the similarity of regulation in the latter experiment for DEGs in mice fed the chow diet or purified diet based on starch detected in the initial experiment.

E. coli ΔidnK/ΔgntK In Vivo Competition Assay

One group of germ-free C57BL/6 (J) mice was switched to the starch-based diet 48 h before gavage whereas the other remained on the standard chow diet. E. coli MG1655 (wt, SRA accession number provided upon publication) was grown in 200 ml of LB and E. coli MG1655 Str^{R} ΔidnK/ΔgntK in 250 ml of LB with 30 μg/ml of chloramphenicol (Sigma-Aldrich), at 37° C., 180 rpm overnight. Cultures were pelleted by centrifugation at 3480 g for 10 min at room temperature, washed twice with 250 ml of sterile PBS and combined after the first washing step at a 1:1 ratio. The combined E. coli strains were finally resuspended in 7.5 ml of sterile PBS and diluted 1:10 in sterile PBS to gavage˜1·10⁹ CFU per mouse in 500 μl. Feces were collected at 24 hours, 48 hours, and 72 hours after gavage and lysed in 1 ml of PBS using a Retsch MM400 tissue lyser at 30 Hz for 3 min. Serial dilutions were streaked onto LB agar and LB agar containing 30 μg/ml of chloramphenicol and the competitive indices were calculated as CFU_{ΔidnK/Δgntk}/CFU_{wt}=CFU_{ΔidnK/Δgntk}/(CFUₜₒₜₐₗ−CFU_{ΔidnK/Δgntk}).

E. coli ΔuxaC In Vivo Competition Assay

The competition experiment using the ΔuxaC mutant was done as above with the following alterations: E. coli MG1655 Str^{R} ΔuxaC (SRA accession number provided upon publication) was gavaged into germ-free mice together with either MG1655 wild type (SRA accession number provided upon publication) or with MG1655 Str^{R} (SRA accession number provided upon publication). Each strain was grown in 100 ml of LB—the ΔuxaC mutant with 50 μg/ml of kanamycin sulfate. After washing with 100 ml of PBS and mixing of the strains, bacteria were resuspended in a final volume of 33 ml to achieve a gavage dose of ˜1·10⁹ CFU per mouse in 500 μl. Serial dilutions of feces were streaked onto LB agar with and without 50 μg/ml of kanamycin sulfate.

### Record-Seq and RNA-Seq Assessment of Different Anatomical Sections of the Murine Gut

One group of germ-free C57BL/6 (J) mice was switched to the starch-based diet 48 hours before gavage whereas the other remained on the standard chow diet. Mice received drinking water containing 30 μg/ml of aTc and 100 μg/ml of kanamycin sulfate water and were gavaged with 1·10⁹ cells of E. coli MG1655 transformed with pFS_0453 as described above. Record-seq plasmid and/or fecal RNA was extracted from individual mouse feces collected daily. On day 7 after gavage, mice were sacrificed and E. coli RNA was collected from various intestinal sections of individual mice: cecal contents were mixed in a Petri dish to homogenize and ˜100 mg collected for RNA extraction. Proximal colon contents were collected at 1-2 cm distance from the cecum. Distal colon contents were collected in the terminal 2 cm of the colon. RNA was extracted, sequenced and analysed as described above. The experiment was performed twice: once with samples being pooled from each three individual mice, once with sampling from individual mice.

Primary analysis was performed for RNA-seq readout as described above for both experiments. During secondary analysis, the minimum value of transcript-aligning counts was set to 100,000. Differential expression analysis between the diet groups was performed for each intestinal section (cecum, proximal colon and distal colon) independently. Record-seq DEGs were overlapped with DEGs identified through RNA-seq proximally (cecum or proximal colon) or distally (distal colon) (P_{adj (section)}<0.1; log₂FC_{Record-seq}×log₂FC_{section}>0). Differential expression analysis between the intestinal sections was performed for each diet group independently. Genes enriched in a particular intestinal section were defined as an overlap of genes identified as upregulated in that section compared to the other two sections (e.g. genes enriched in the cecum on the chow diet were defined as genes that were upregulated in the cecum on the chow diet compared to both the proximal and distal colon). Rank-based normalization was used for comparing Record-seq and RNA-seq counts to account for the differences in count distributions.

In Vitro Exposure of E. coli to Dextran Sodium Sulfate (DSS)

For each replicate, three colonies of E. coli MG1655 transformed with pFS_0453 were inoculated into 2 ml of terrific broth (TB) (24 g per liter of yeast extract, 20 g per liter of tryptone, 4 ml per liter of glycerol, 17 mM of KH₂PO₄, 72 mM of K₂HPO₄) containing 50 ng/ml of aTc and 0.1, 0.3, 1, 3 or 10% (w/v) of DSS. After overnight culture at 37° C., 220 rpm bacterial cultures were pelleted and plasmid DNA extracted using 500 μl of buffer P1, 500 μl of buffer P2 and 700 μl of buffer N3, wash steps were carried out as described above. A single elution was performed by adding 60 μl of buffer TE, followed by incubation at 55° C., 850 rpm for 1 min and centrifugation at 20,000 g, for 1 min at room temperature. Plasmid DNA was quantified as described (T. Tanna et al., 2020, ibid), and input normalized for SENECA using annealed adapter ligation oligonucleotides FS_2108 and FS_2109 (Table 4). Primary and secondary analysis of data was performed as described above, with the minimum value of cumulative transcript-aligning spacer counts per sample (C) set to 10,000.

### Record-Seq Assessment of DSS Colitis In Vivo

Germ-free C57BL/6 (J) mice received drinking water containing 30 μg/ml of aTc and 100 μg/ml of kanamycin sulfate water and were gavaged with 1·10⁹ cells of E. coli MG1655 or E. coli BL21 (DE3) transformed with pFS_0453 as described above. Dextran sulfate sodium (DSS, molecular weight 36-50 kDa, Lucerna-Chem) was dissolved in tap water to 1, 2, or 3% (w/v) with aTc and kanamycin sulfate as above. Mice received DSS-containing drinking water for 5 days as illustrated in the experimental outlines. Subsequently, mice were switched back to aTc and kanamycin sulfate-containing water without DSS. Mice were monitored daily for signs of distress. Mice treated with 3% DSS (w/v) in the drinking water had to be removed from the study on day 13 of the experiment due to colitis-induced distress and are thus omitted from the analysis for days 14 to 19 in panels 4B and 4C. Plasmid DNA from fecal pellets was extracted and concentrated as outlined above.

The 2% DSS (w/v) experiment (FIG. 4D) with E. coli MG1655 used 3.75 μl of plasmid DNA as an input to SENECA using annealed adapter ligation oligonucleotides FS_2806 and FS_2807 (Table 4). The initial experiment, including different concentrations of DSS (1%, 2%, 3% w/v) with E. coli BL21 (DE3) used 7.5 μl of plasmid DNA as an input to SENECA using the annealed adapter ligation oligonucleotides FS_2238 and FS_2246 (Table 4).

Primary analysis of the Record-seq sequencing readout for both experiments was performed as described above. For the initial experiment (FIG. 4A) using E. coli BL21 (DE3), the minimum value of C was set to 5,000 counts. Hierarchical clustering and heatmap generation using DEGs was performed for day 19. For inferring the trajectory of DSS-induced differences between treatment groups using PCA-reduced data from days 5 to 9 (FIG. 12D), an approach analogous to Slingshot (K. Street et al., BMC Genomics 19, 477 (2018)) was employed: clusters were defined using k-medoids clustering with an optimal k value (k=5) determined by the Elbow method based on minimizing within-cluster sum of squares. Branching of trajectories was inferred based on the assumption that clusters with samples from earlier timepoints branch out into clusters with samples from later timepoints, with the nodes of the plotted trajectory indicating the center of each cluster. For discriminating between treatment groups, Record-seq data on days 6-19 (treatment and post-treatment) was randomly split into a 70% training and a 30% test set using a fixed random seed to ensure reproducibility. One-vs-rest classification was performed on the test sets with SVM models trained and tuned on the training sets using leave-one-out cross-validation implemented in the e1071 package in R. Receiver operating characteristic (ROC) curves were generated using the ROCR package in R.

For secondary analysis, in the 2% DSS experiment (E. coli MG1655), the threshold for C was set to 10000 counts for Record-seq. PCAs and k-medoids clustering were performed for Record-seq samples collected post DSS treatment. k-medoids cluster identity was encoded by convex hulls in PCA plots. UMAP and time-course log₂FC plots was generated and differential expression was performed using days 2 to 20, and identified DEGs were used for EcoCyc pathway enrichment and STRING network analysis using Cytoscape. MCL clustering was performed with the granularity parameter set at 2.5 and stringdb::score was used as array source with an edge cutoff of 0.5 to identify groups in the STRING networks. Clusters containing more than 2 genes were reported. KEGG- and GO-based OA were performed for DEGs using clusterProfiler.

Record-Seq in the Presence or Absence of Bacteroides thetaiotaomicron (B. thetaiotaomicron)

Germ-free C57BL/6 (J) mice received the standard chow diet throughout the entire experiment, drinking water containing 30 μg/ml of aTc and 100 μg/ml of kanamycin sulfate from the day before gavage with 1·10⁹ cells of E. coli MG1655 transformed with pFS_0453 as described above. For oral gavage of Bacteroides thetaiotaomicron (B. thetaiotaomicron) a single colony of B. thetaiotaomicron was grown overnight in 140 ml of brain heart infusion broth (Thermo Fisher Scientific) supplemented with 0.5 milligram per liter of menadione and 5 milligram per liter of hemin (both Sigma-Aldrich) at 37° C. under anaerobic conditions without agitation. After two wash steps with 150 ml of sterile anaerobic PBS, B. thetaiotaomicron was resuspended and mixed with E. coli suspension to gavage each 1·10⁹ CFU E. coli and 1·10⁹ CFU B. thetaiotaomicron in a total volume of 500 μl. Plasmid DNA was extracted from fecal samples using the procedure outlined above but increasing the buffer volumes to 1000 μl of buffer P1, 1000 μl of buffer P2 and 1400 μl of buffer N3. 2-propanol precipitation and resuspension were performed as described above. The 9-day experiment used 3.75 μl of plasmid DNA as an input into the SENECA using annealed adapter ligation oligonucleotides FS_2762 and FS_2772 (Table 4). The 27-day experiment used 3.75 μl of plasmid DNA as an input and annealed adapter ligation oligonucleotides FS_2758 and FS_2768 (Table 4).

For measurement of bacterial colonization levels, feces were collected, weighed and lysed in 1 ml of PBS using a Retsch MM400 tissue lyser at 30 Hz for 3 min. Serial dilutions were streaked onto LB agar plates to determine E. coli CFU and on BHI agar with 5% defibrinated sheep blood to determine B. thetaiotaomicron CFU and incubated aerobically or anaerobically, respectively.

Primary analysis of data was performed for Record-seq data as described above for both experiments. For secondary analysis, the minimum value of C was set to 5,000. This lower threshold for cumulative transcript-aligning spacer counts was chosen due to fewer observed counts, possibly explained by the difficulty in plasmid DNA extraction from E. coli colonized with B. thetaiotaomicron. Hierarchical clustering was performed and heatmaps were generated for the full time-course using DEGs detected on at least 2 days. Combined lists of DEGs identified on any day were used for subsequent EcoCyc pathway enrichment, STRING network analysis using Cytoscape, and KEGG- and GO-based OA using clusterProfiler. The granularity parameter for MCL clustering of STRING networks was set at 2.5 and stringdb::score was used as array source with an edge cutoff of 0.5. Clusters containing more than 3 genes were reported.

Record-Seq Function in Complex Microbiomes (sDMDMm2 Mice)

C57BL/6 (J) mice with the sDMDMm2 microbiota (Table 2) received the standard chow diet or the starch-based purified diet (Research Diets D12450Jii) and were switched to drinking water containing 30 μg/ml of aTc but no kanamycin sulfate 6 days before gavage. The gavage procedure was modified as follows: 400 ml of an overnight culture of E. coli MG1655 cells transformed with pFS_0453 in LB with 50 μg/ml of kanamycin sulfate were diluted 1:5 into 2 l pre-warmed LB with 30 ng/μl of aTc and 50 μg/ml of kanamycin sulfate and cultured for another 2 h. Bacteria were pelleted by centrifugation at 3480 g for 10 min at room temperature, washed twice with 1 L PBS and resuspended in 12 ml to gavage 6.101° CFU into each mouse in 500 μl of PBS. Plasmid DNA was extracted from fecal samples using the procedure outlined above but increasing the buffer volumes to 1000 μl of buffer P1, 1000 μl of buffer P2 and 1400 μl of buffer N3. Isopropanol precipitation and resuspension were performed as described above. SENECA adapter ligation was performed using 3.75 μl of plasmid DNA and annealed adapter ligation oligonucleotides FS_2760 and FS_2770 as well as FS_2761 and FS_2771 (Table 4) with the following modification compared to the standard protocol: annealed adapter ligation oligonucleotides were diluted 1:10 instead of 1:100 in TE buffer after annealing. E. coli colonization levels were measured by homogenizing and serial dilution of fecal pellets as described for the B. thetaiotaomicron experiment above but serial dilutions were spread on MacConkey agar (Thermo Fisher Scientific) with 50 μg/ml kanamycin sulfate to ensure selective growth of E. coli sentinel cells.

Primary analysis of data was performed for Record-seq data as described above for both experiments. For secondary analysis, the minimum value of C was set to 5000. Further, since the genome-aligning spacer counts increased over the sampling time course, data from the final 21 h timepoint was used for identifying DEGs, hierarchical clustering and heatmap generation, EcoCyc pathway enrichment, STRING network analysis using Cytoscape, and KEGG- and GO-based OA using clusterProfiler. The granularity parameter for MCL clustering of STRING networks was set at 2.5 and stringdb::score was used as array source with an edge cutoff of 0.5. Clusters containing at least two genes were reported.

### Transcription-Stimulated Spacer Acquisition

Transcription-stimulated recording constructs were generated by designing gBlock FS_3046 designed following the supplementary methods of (J. B. Budhathoki et al, Nature structural & molecular biology 27, 489-499 (2020)). gBlock FS_3046 (Table 4) encodes “T7UUCG (terminator)_rrnBT1 (terminator)_T7Term (terminator)_Golden-Gate (Bbsl)_FsLeader-CRISPR-02-array-DR2_50 bp (stuffer)_EK120029600 (terminator_inverted)” and is inserted downstream of FsRT-Cas1-Cas2 in pFS_0453 cloning with XhoI and NotI. This yields a plasmid where oligonucleotides encoding various promoter sequences can be inserted using a Golden Gate reaction with Bbsl. Based on pFS_1061, plasmids pFS_1061 to pFS_1067 as well as pFS_1112 to pFS_1114 were generated. These plasmids encode constitutive E. coli promoters of different transcriptional activity upstream of the FsLeader_CRISPR_02_array. These promoters are inserted into pFS_1061 by generating double stranded (dsDNA) fragments encoding the respective promoter and appropriate overhangs through the annealing of oligonucleotides FS_3047 and FS_3048 (for pFS_1062), FS_3049 and FS_3050 (for pFS_1063), FS_3051 and FS_3052 (for pFS_1064), FS_3053 and FS_3054 (for pFS_1065), FS_3055 and FS_3056 (for pFS_1066), FS_3057 and FS_3058 (for pFS_1067), FS_3210 and FS_3211 (for pFS_1112), FS_3212 and FS_3213 (for pFS_1113), FS_3214 and FS_3215 (for pFS_1114). Sequence of oligonucleotides are available in table 4. Oligonucleotides were annealed by mixing 2.5 μl of 100 μl oligonucleotides in buffer TE with 5 μl of NEBuffer 2.0 and 40 μl of Ultrapure H₂O (ThermoFisher Scientific) per reaction. The reaction was heated to 95° C. for 5 min in a thermocycler and cooled to 22° C. at a rate of 0.5° C./sec. Then the annealed oligonucleotides were diluted 1:200 in Ultrapure H₂O. For each target plasmid a Golden Gate reaction was performed containing 40 fmol of pFS_1061, 1 μl of 1:200 diluted, annealed oligonucleotides, 1 μl of a mixture of ATP and DTT (10 mM each) (ThermoFisher Scientific), 0.25 μl of T7 DNA Ligase (NEB), 0.75 μl of 40% (w/v) PEG8000 (Sigma-Aldrich), 0.75 μl of Bpil (ThermoFisher Scientific), 1 μl of buffer green (ThermoFisher Scientific). Each reaction was filled up to 10 μl total volume using Ultrapure H₂O (ThermoFisher Scientific).

From each Golden Gate reaction, 0.5 μl were transformed into 5 μl of chemically competent E. coli Stbl3. Individual clones were grown in LB media containing 50 μg/ml of kanamycin sulfate, isolated by plasmid mini-prep and validated by Sanger sequencing (GATC Eurofins). Correct clones were then transformed into chemically competent E. coli MG1655 to assess efficiency of recording. In vitro recording and SENECA reaction was performed as described previously (F. Schmidt et al., Nature 562, 380-385 (2018)). Upon identification of J23103 (iGEM Registry of Standard Biological Parts (http://parts.igem.org)) gBlock FS_3344 encoding “T7UUCG (terminator)_rrnBT1 (terminator)_T7Term (terminator)_J23103_FsLeader-CRISPR-01-array-DR1_50 bp (stuffer)_EK120029600 (terminator_inverted)” was designed and cloned into pFS_0453 using XhoI and NotI, yielding pFS_1142.

### Record-Seq for Multiplexed Recording in Different Bacterial Chassis

For in vivo multiplexed recording experiments pFS_1113 and pFS_1142 were transformed into chemically competent MG1655 Str^{R} ΔuxaC (“ΔuxaC”, SRA accession number provided upon publication) or MG1655 Str^{R} NalR (“wt”, SRA accession number provided upon publication) and spread on LB-Agar plates containing 50 μg/ml of kanamycin sulfate. Germ-free C57BL/6 (J) mice were switched to the starch-based diet 72 hours before gavage and received drinking water containing 30 μg/ml of aTc and 100 μg/ml of kanamycin sulfate from 24 hours before gavage onwards. All E. coli strains were inoculated into LB medium with 100 μg/ml of streptomycin sulfate and 50 μg/ml of kanamycin sulfate with or without 50 μg/ml of nalidixic acid, respectively and grown over night at 37° C. with 180 RPM shaking. Following washing as described above, the following strains were mixed 1:1 and a total of 1·10¹⁰ CFU (5·10⁹ CFU per strain and mouse) were orally gavaged into recipient mice. (A) wt-pFS_1113 together with ΔuxaC pFS_1142 and (B) wt-pFS_1142 together with ΔuxaC pFS_1113. Colonisation levels of the strains were determined by fecal dilution and plating on LB/Str100/Kan50 (both ΔuxaC and wt) and LB/Str100/Kan50/NaI50 (only wt).

Plasmid DNA was extracted from fecal samples using the procedure outlined above but increasing the buffer volumes to 500 μl of buffer P1, 500 μl of buffer P2 and 700 μl of buffer N3. 2-propanol precipitation and resuspension were performed as described above.

SENECA adapter ligation was performed using 7.5 μl of plasmid DNA and annealed adapter ligation with annealed oligonucleotides FS_3194 and 3204 as well as FS_3316 and 3321 (Table 4) with the following modification compared to the standard protocol: annealed adapter ligation oligonucleotides were diluted 1:10 instead of 1:100 in TE buffer after annealing. Upon annealing to dsDNA fragments oligonucleotides FS_3194 and 3204 form an overhang compatible with FaqI digested FsDR2 and oligonucleotides FS_3316 and 3321 form an overhang compatible with FaqI digested FsDR1. Therefore, both sets of annealed oligos are used in a single SENECA adapter ligation reaction to simultaneously read out CRISPR spacer acquisition into pFS_1113 and pFS_1142. Accordingly, SENECA first round PCR was performed with FS_968, FS_969, FS_970, FS_971, FS_972, FS_973, FS_974 described previously (F. Schmidt et al., 2018, ibid) which are primers compatible with FsDR2 as well as primers compatible with FsDR1, namely FS_3325, FS_3326, FS_3327, FS_3328, FS_3329, FS_3330, FS_3331 (Table 4) each at a concentration of 0.714 UM and the universal reverse primer FS_911 at a concentration of 10 μM. SENECA second round PCR was performed as described above.

For primary analysis of data, a modified version of the Snakemake workflow described above was used. This workflow requires the additional input of a table with sample-specific entries for the reference plasmid, DR sequence and library barcode. Samples with multiple DR-barcoded E. coli strains, such as those from the in vivo multiplexed recording experiment, resulted in reads containing both DR sequences (FsDR2 and FsDR1). These were stratified into strain-specific reads by identifying the DR-specific library barcode using fuzzy string matching and processed independently. Secondary analysis was performed as described above.

For the in vitro multiplexed recording experiment, the minimum value of cumulative transcript-aligning spacer counts (C) was set to 30,000. For the in vivo multiplexed recording experiment, the minimum value of cumulative transcript-aligning spacer counts (C) was set to 10,000. DEGs were identified between the strains (wt vs ΔuxaC) for days 7 to 10 in both groups of mice independently (group 1: ΔuxaC-DR1, wt-DR2; group 2: wt-DR1, ΔuxaC-DR2; n=5 in each group). High-confidence DEGs were defined as DEGs identified as regulated in the same direction for at least 3 out of 4 days in both comparisons. These high-confidence DEGs were used for EcoCyc pathway enrichment and KEGG- and GO-based OA using clusterProfiler.

### Record-Seq in Highly Presence of Complex Microbiota

Specific pathogen free (SPF) C57BL/6 (J) mice received the standard chow diet or the starch-based purified diet (Research diets D12450Jii) and were switched to drinking water containing 30 μg/mL of aTc but no kanamycin sulfate 6 days prior to gavage. The gavage procedure was conducted as follows: 30 mL of an overnight culture of E. coli MG1655 cells transformed with pFS_1113 in LB with 50 μg/ml of Kanamycin sulfate were diluted 1:20 into a total volume of 600 ml LB with 50 μg/ml of kanamycin and incubated for 7.5 h at 37° C. and 200 rpm. The 600 ml culture was diluted 1:5 into a total volume of 3 liters pre-warmed LB with 30 ng/μL of anhydrotetracycline and 50 μg/ml of Kanamycin to induce expression of FsRT-Cas1-Cas2. This culture was incubated for another 2 h at 37° C., 200 rpm. Bacteria were then pelleted by centrifugation at 3480×g for 10 min at room temperature, washed twice with 1 L of PBS and resuspended in 8 mL of PBS to gavage 9.4×10E+10 CFU into each mouse in 500 UL of PBS. Fecal samples were collected and frozen at 12 h, 15 h, 18 h, 21 h and 24 h post gavage and stored at −20° C. After thawing, feces or intestinal contents were homogenized in 1 ml of PBS using a Retsch MM400 tissue lyser at 30 Hz for 3 min. Large particles were pelleted by centrifugation at 200×g for 2 min at room temperature. Bacteria in the supernatant were pelleted by centrifugation at 6,800×g for 3 min at room temperature and plasmid DNA was isolated using the QIAprep spin Miniprep kit (QIAGEN) but increasing the buffer volumes to 1000 μL of buffer P1, 1000 UL of buffer P2 and 1400 UL of buffer N3. A total of three elution steps with 50 μL of EB buffer were performed yielding 150 μL of eluate. DNA from this eluate was further concentrated by precipitation. For this, 150 μL of 2-propanol and 15 μL of 3 M sodium acetate solution were added to each sample, followed by incubation at −20° C. for 20 min and centrifugation at 20,000×g for 20 min at room temperature. Supernatant was carefully removed without disturbing the pellet. The pellet was then washed with 500 μL of 80% (v/v) ethanol and centrifuged at 20,000×g for 15 min at room temperature. Ethanol was removed completely and the pellet was briefly dried (55° C., 30 s) before resuspension in 15 μL of buffer EB and transfer to a 96-well DNA LoBind PCR plate (Eppendorf) and storage at −20° C. or immediate use in SENECA. SENECA adapter ligation was performed using 7.5 μL of precipitated plasmid DNA and annealed adapter ligation oligonucleotides FS_3195 and FS_3205. Annealed adapter ligation oligonucleotides were diluted 1:10 in ultrapure water after annealing. First round SENECA PCR was performed with 20 cycles, second round PCR with 9 cycles.

E. coli colonization levels were measured by homogenizing and serial dilution of fecal pellets. Serial dilutions were spread on MacConkey agar with 50 μg/mL of kanamycin sulfate to ensure selective growth of E. coli sentinel cells.

Sequencing reads were pre-processed using FastQC v0.11.4 and trimmomatic v0.35. FASTQ files containing sequencing results were converted to FASTA files using FASTX-toolkit v0.0.14. Reads without the library barcode were excluded and unique spacer sequences were identified and quantified from the remaining reads using an in-house python script. Identified unique spacer sequences were aligned to the reference E. coli str. K-12 substr. MG1655 genome (GenBank U00096.3) using Bowtie 2. Duplicate alignments were removed using a custom python script. Count matrices were generated by quantifying alignments using featureCounts from the Subread package. All the steps of this pipeline were implemented in a Snakemake workflow. Secondary analysis was performed on generated count matrices using the recoRdseq package in R. Unsupervised clustering of samples and identification of classifier genes based on differential expression analysis was performed. A counts threshold of 5000 was used to exclude samples with a low number of genome-aligning spacer counts.

Sequences
SEQ ID NO 01
GTTGTACCTTACCTATGAGGAATTGAAAC
SEQ ID NO 02
GTCGTACTTTACCTAAAAGGAATTGAAAC
SEQ ID NO 03
GTAAAACTTTACCTAAAAGGAATTGAAAC
SEQ ID NO 04
GTCGGTCTTTACCTAAAAGGAATTGAAAC
SEQ ID NO 05
GTAAATCTTTACCTAAAAGGAATTGAAAC
SEQ ID NO 06
GTCAAACTTTACCTAAAAGGAATTGAAAC
SEQ ID NO 07
GTACGGCTTTACCTAAAAGGAATTGAAAC
SEQ ID NO 08
GTAGGTCTTTACCTAAAAGGAATTGAAAC
SEQ ID NO 09
GTTAGTCTTTACCTAAAAGGAATTGAAAC
SEQ ID NO 10
GTTACGCTTTACCTAAAAGGAATTGAAAC
SEQ ID NO 11
GTCACACTTTACCTAAAAGGAATTGAAAC
SEQ ID NO 12
AAAGCAAGCCCGTTCACAACTACGNNNNNNNNNN
GATCGGAAGAGCACACGTCTGAACTCCAGTCAC
SEQ ID NO 13
CGTAGTTGTGAACGGGCTTG

PCT/EP2019/074267, published as WO2020053299A1 and US20220049232A1 (Ser. No. 17/274,443, which is incorporated by reference herein in its entirety) discloses sequences useful for practicing the invention, particularly the sequences identified by the identifier numbers 001 to 102 according to the numbering of that application.

### CITED PUBLICATIONS

Shipman et al. (Science 353 (6298), 2016 (https://doi.org/10.1126/science.aaf1175)
Sheth et al., (Science 358; 1457-1461 (2017) (https://doi.org/10.1126/science.aao0958)
Wang et al., Nature Communications volume 12, Article number: 2571 (2021)
F. Schmidt, M. Y. Cherepkova, R. J. Platt, Transcriptional recording by CRISPR spacer acquisition from RNA. Nature 562, 380-385 (2018)
T. Tanna, F. Schmidt, M. Y. Cherepkova, M. Okoniewski, R. J. Platt, Recording transcriptional histories using Record-seq. Nature protocols 15, 513-539 (2020)
K. A. Datsenko, B. L. Wanner, One-step inactivation of chromosomal genes in Escherichia coli K-12 using PCR products. Proceedings of the National Academy of Sciences of the United States of America 97, 6640-6645 (2000)
B. Langmead, S. L. Salzberg, Fast gapped-read alignment with Bowtie 2. Nature methods 9, 357-359 (2012)
H. Li et al., The Sequence Alignment/Map format and SAMtools. Bioinformatics (Oxford, England) 25, 2078-2079 (2009)
Y. Liao, G. K. Smyth, W. Shi, featureCounts: an efficient general purpose program for assigning sequence reads to genomic features. Bioinformatics (Oxford, England) 30, 923-930 (2014)
J. Koster, S. Rahmann, Snakemake—a scalable bioinformatics workflow engine. Bioinformatics (Oxford, England) 28, 2520-2522 (2012)
M. I. Love, W. Huber, S. Anders, Moderated estimation of fold change and dispersion for RNA-seq data with DESeq2. Genome Biol 15, 550 (2014)
L. Mclnnes, J. Healy, J. Melville, UMAP: Uniform Manifold Approximation and Projection for Dimension Reduction. 2018
N. T. Doncheva, J. H. Morris, J. Gorodkin, L. J. Jensen, Cytoscape StringApp: Network Analysis and Visualization of Proteomics Data. J Proteome Res 18, 623-632 (2019)
J. H. Morris et al., clusterMaker: a multi-algorithm clustering plugin for Cytoscape. BMC bioinformatics 12, 436 (2011)
M. Kanehisa, M. Furumichi, Y. Sato, M. Ishiguro-Watanabe, M. Tanabe, KEGG: integrating viruses and cellular organisms. Nucleic acids research 49, D545-D551 (2021)
G. Yu, L. G. Wang, Y. Han, Q. Y. He, clusterProfiler: an R package for comparing biological themes among gene clusters. OMICS 16, 284-287 (2012)
K. Street et al., Slingshot: cell lineage and pseudotime inference for single-cell transcriptomics. BMC Genomics 19, 477 (2018)
WO2020053299A1

1. A cell comprising
i. a first transgene nucleic acid sequence encoding a fusion protein comprising or essentially consisting of a reverse transcriptase polypeptide and a Cas1 polypeptide,
ii. a second transgene nucleic acid sequence encoding a Cas2 polypeptide, and
iii. a third transgene nucleic acid sequence comprising a CRISPR direct repeat sequence (DR sequence) and a CRISPR leader sequence;
wherein said DR sequence and said CRISPR leader sequence are specifically recognizable by an RT-Cas1-Cas2 complex formed by the expression products of said first transgene nucleic acid sequence and said second transgene nucleic acid sequence, and
wherein a third transgene promoter in addition to the leader sequence,
particularly a weak third transgene promoter, more particularly a weak constitutive third transgene promoter specific for RNA polymerase,
is associated with (located in proximity of <1 kbp, particularly <500 bp), particularly located in 5′ direction of, the DR sequence.

2. The cell according to claim 1, wherein said third transgene promoter is selected from the group comprising pTrc, BBa_J23100, BBa_J23106, BBa_J23110, BBa_J23115, BBa_J23117, BBa_J23109, BBa_J23112.

3. A composition comprising
a first cell as specified in claim 1, and
a second cell as specified in claim 1,
wherein
i. the third transgene nucleic acid sequence comprised in the first cell comprises a first CRISPR direct repeat sequence (first DR sequence) and a CRISPR leader sequence; and
ii. the third transgene nucleic acid sequence comprised in the second cell comprises a second CRISPR direct repeat sequence (second DR sequence) and a CRISPR leader sequence; and
wherein the first and the second DR sequences differ in at least one nucleotide.

4. The composition according to claim 3, wherein the first DR sequence is SEQ ID NO 01 (GTTGTACCTTACCTATGAGGAATTGAAAC) and the second DR sequence is SEQ ID NO 02 (GTCGTACTTTACCTAAAAGGAATTGAAAC).

5. The composition according to claim 3, wherein the first DR sequence is SEQ ID NO 01 or SEQ ID NO 02 and the second DR sequence differs from the first DR sequence in at least one nucleotide, particularly in 4, 3, 2, or 1 nucleotide(s), more particularly in two nucleotides.

6. The composition according to claim 3, wherein the first and the second DR sequence are selected from different sequences of the group of SEQ ID NO 01 to SEQ ID NO 11.

7. The composition according to claim 3, wherein the first and the second cell are of the same species, particularly of the species E. coli.

8. The composition according to claim 7, wherein the first and the second cell differ in expression of at least one gene, particularly wherein the one gene encodes an enzyme catalyzing an essential metabolic step.

9. The cell or the composition according to claim 1 for use in diagnosis.

10. A method for monitoring of a diet of a patient or for diagnosis of a disease of a patient, particularly of a digestive or gastrointestinal disorder of a patient, said method comprising the steps of
collecting a cell according to claim 1, or DNA from said cell, from a feces sample collected from said patient, wherein said cell had been previously applied orally to said patient,
isolating the third transgene nucleic acid sequence from said cell, yielding an isolated third transgene nucleic acid sequence, and
sequencing said isolated third transgene nucleic acid sequence.
thereby recording a transcript of said cell produced in the environment of the gastrointestinal tract.

11. A method for monitoring of a diet of a patient or for diagnosis of a disease of a patient, particularly of a digestive or gastrointestinal disorder of a patient, said method comprising the steps of
collecting a composition of cells according to claim 3, or DNA from said composition of cells, from a feces sample collected from said patient, wherein said cells had been previously applied orally to said patient,
isolating the third transgene nucleic acid sequence from said cells, or amplifying said third transgene sequence from said DNA, yielding isolated third transgene nucleic acid sequences, and
sequencing said isolated third transgene nucleic acid sequences, and distinguishing said isolated third transgene nucleic acid sequences derived from the first cell from the said isolated third transgene nucleic acid sequences derived from the second cell by the difference in the first and second DR sequence;
thereby recording one or more transcripts of said composition of cells produced in the environment of the gastrointestinal tract.

12. A method for diagnosis of a condition affecting the intestine of a patient, said method comprising the steps:
i. collecting cells, or DNA from said cells, from a feces sample collected from the patient, wherein said cells had previously been applied orally to said patient;
wherein the cells prior to the oral application comprised:
a first transgene nucleic acid sequence encoding a fusion protein comprising or essentially consisting of a reverse transcriptase polypeptide and a Cas1 polypeptide and a second transgene nucleic acid sequence encoding a Cas2 polypeptide, said first transgene nucleic acid sequence and said second transgene nucleic acid sequence being under transcriptional control of an, optionally inducible, promoter sequence, and
a third transgene nucleic acid sequence comprising a CRISPR direct repeat sequence; said CRISPR direct repeat sequence being specifically recognizable by an RT-Cas1-Cas2 complex formed by the expression products of said first transgene nucleic acid sequence and said second transgene nucleic acid sequence,
wherein in the collected cells, spacers derived from a plurality of RNA molecules of the cell have been integrated into the CRISPR direct repeat sequence, yielding a modified third transgene nucleic acid sequence;
ii. isolating the modified third transgene nucleic acid sequence from said cells, or amplifying said modified third transgene nucleic acid sequence from said DNA, yielding an isolated modified third transgene nucleic acid sequence, and
iii. sequencing said isolated modified third transgene nucleic acid sequence;
wherein a high probability of having a condition affecting the intestine is assigned to said patient if,
when compared to a plurality of reference cells collected from a subject without said condition affecting the intestine
or
after longitudinal studies of single patients before and following dietary or therapeutic interventions (such as correction or dietary deficiencies, prescription of diets lacking intolerance components, anti-inflammatory treatments, prokinetic administration, antibiotic usage, stool transplantation),
the modified third transgene nucleic acid sequences isolated from said patient comprise spacers derived from an indicator gene,
A) wherein the condition relates to the nutritional status of the patient (macronutrient insufficiency or excess; caloric adequacy after supplementation in malnutrition; micronutrient vitamin and mineral insufficiency), oxidative stress, intestinal inflammation, dysbiosis interaction between different taxa within the intestinal microbiota, niche adequacy for individual microbiota taxa, carbohydrate and lipid malabsorption (functional lactase, sucrase/isomaltase, trehalase and pancreas exocrine deficiencies), control for presence of gluten in diet of celiac disease patients, control for presence of FODMAPs (fermentable oligosaccharides, disaccharides, monosaccharides, and polyols) in diet of patients with intestinal functional disorders and bloating), bile salt malabsorption, delayed intestinal transit disorders (sclerosing conditions such as systemic sclerosis, neuromuscular disorders of the intestinal tract, intestinal changes in autonomic neuropathy of enteric neuropathies);
particularly wherein the condition is malnutrition,
and wherein the indicator gene is selected from the list comprising the following genes: eda; edd; gatYZ; gntKPTU; idnKP; kdgKT; kduDI; nagABCEKZ; nanACEKMQRSTXY; nirB; uxaABC; uxuABC; zraP; alaE; dmsABCD; gadABC; gadABCE; hdeABD; hyaABC; lacAZ; napABCDFGH; narGIJKUWYZ;
B) wherein the condition is intestinal inflammation;
and the indicator gene is selected from the list comprising the following genes:
alaE; ipbAB; lon; mqsAR; pspABCDEGH; spy; tcdABCDEFG; tomB; adhE; dmsABCD; eda; edd; gadABCE; gntK; hdeABD; napABCDFGH; narGHIJKUWYZ; gntTU; kdgKT; nagABCEKZ; nanACEKMQRSTXY; uxaABC.

13. The method according to claim 12, wherein a high probability of malnutrition is assigned to said patient if the modified third transgene nucleic acid sequences isolated from said patient, compared to sequences obtained from reference cells, comprises
a significantly higher amount of a spacer derived from a gene selected from the list comprising eda; edd; gatYZ; gntKPTU; idnKP; kdgKT; kduDI; nagABCEKZ; nanACEKMQRSTXY; nirB; uxaABC; uxuABC; zraP and/or
a significantly lower amount of a spacer derived from a gene selected from the list comprising alaE; dmsABCD; gadABC; gadABCE; hdeABD; hyaABC; lacAZ; napABCDFGH; narGIJKUWYZ.

14. The method according to claim 12, wherein the condition is inflammation of the intestine, and wherein a high probability of the patient suffering from intestinal inflammation is assigned to said patient if, when compared to a reference collected from a subject without malnutrition, the isolated modified third transgene nucleic acid sequences comprise a significantly different amount of spacers derived from genes selected from the list:
alaE; acrZ; bhsA; cpxP; glgS; Hha; ibpA; ibpB; lon; mqsA; mqsR; osmB; pspA; pspB; pspC; pspD; pspE; pspG; pspH; spy; tdcA; tdcB; tdcC; tdcD; tdcE; tdcF; tdcG; tomB; adhE; dmsA; dmsB; dmsC; dmsD; eda; edd; gadA; gadB; gadC; gadE; gntK; hdeA; hdeB; hdeD; hyaA; hyaB; hyaC; napA; napB; napC; napD; napF; napG; napH; narG; narH; narl; narJ; narK; narU; narW; narY; narZ; gntP; gntT; gntU; idnK; idnP; kdgK; kdgT; kduD; kdul; nagA; nagB; nagC; nagE; nagK; nagZ; nanA; nanC; nanE; nanK; nanM; nanQ; nanR; nanS; nanT; nanx; nanY; uxaA; uxaB; uxaC; uxuA; uxuB; uxuC.

15. An isolated nucleic acid molecule comprising a direct repeat sequence selected from the group comprising SEQ ID NO 1, SEQ ID NO 02, SEQ ID NO 03, SEQ ID NO 04, SEQ ID NO 05, SEQ ID NO 06, SEQ ID NO 07, SEQ ID NO 08, SEQ ID NO 09, SEQ ID NO 10, SEQ ID NO 11.
